(19) United States
(12) Patent Application Publication
(10) Pub. No.: US 2021/0228632 A1
(43) Pub. Date: Jul. 29, 2021

(54) COMPOSTION FOR USE IN IMMUNOTHERAPY

(71) Applicant: GLYCOSTEM THERAPEUTICS B.V., Oss (NL)

(72) Inventors: Wim Jongen, Oss (NL); Jan Spanholtz, Oss (NL)

(73) Assignee: GLYCOSTEM THERAPEUTICS B.V., Oss (NL)

(21) Appl. No.: 17/217,195

(22) Filed: Mar. 30, 2021

Related U.S. Application Data
(63) Continuation of application No. 15/773,610, filed on May 4, 2018.

(30) Foreign Application Priority Data
Nov. 5, 2015 (NL) 2015728
Sep. 9, 2016 (NL) 2017439

(51) Int. Cl. A61K 35/17; A61P 35/02; A61K 35/51; C12N 5/0789

(52) U.S. Cl. CPC A61K 35/17; C12N 5/0647; A61K 35/51; A61P 35/02

(57) ABSTRACT

In the fields of immunology and medicine, and more specifically in the fields of cancer treatment and immunotherapy, a composition for use in immunotherapy, in particular in a subject having a tumor is provided. The use of immunosuppressive pharmaceutical compositions is provided, in particular for use prior to immunotherapy. Methods for providing compositions for use in immunotherapy are also provided.

### RELATED APPLICATIONS

This application is a continuation of U.S. Utility application Ser. No. 15/773,610 filed May 4, 2018; which in turn is US National Phase filing of PCT/EP2016/076758 filed Nov. 5, 2016 that in turn claims priority benefit of Dutch Applications N2015728 filed Nov. 5, 2015 and N2017439 filed Sep. 9, 2016; the content of which are hereby incorporated by reference.

### FIELD OF THE INVENTION

The present invention relates to the fields of immunology and medicine. The present invention more specifically relates to the fields of cancer treatment and immunotherapy. The invention further relates to composition for use in immunotherapy, in particular in a subject having a tumor. The invention further relates to the use of immunosuppressive pharmaceutical compositions, in particular for use prior to immunotherapy. The present invention in addition relates to methods for providing compositions for use in immunotherapy.

### BACKGROUND OF THE INVENTION

The formation of all types of cells is crucial to endow humans with various important functions and tissue regeneration. The development of multicellular organisms is mainly dependent on the function of somatic stem cells. These cells are defined as undifferentiated cells, which can self-renew over a long period and give rise to progenitor cells committed to more specific lineages during development. Controlled development and differentiation of stem cells leads to a highly complex functional organ or organ systems. However, uncontrolled differentiation or genetic aberrations in stem cells could lead to death or development of cancer, immunodeficiency, autoimmunity or bone marrow insufficiency.

These malignancies of solid or hematological tumors are generally treated with chemotherapy and radiotherapy. However, drug resistance and relapse remain major problems and allogeneic hematopoietic stem cell transplantation (HSCT) is often the final treatment modality for many of these diseases. Transplantation of HSCs has been extensively used to treat leukemia and other types of cancers^{1,2}. It has been clearly demonstrated that the adult and neonatal HSCs keep the ability to reconstitute the hematopoietic systems of patients after myeloablative treatment³. Therefore, an important feature of HSCs is the capacity to replenish all lineages of mature blood cells.

However, HSCT is still a risky procedure implying various possible complications, such as treatment related mortality due to graft versus host disease (GVHD), graft failures or infections. New medications such as specific drugs, antibodies or various forms of adoptive cellular immunotherapy are under current development to reduce risks of HSCT and to improve the quality of life for the patient.

Since more than 50 years HSCs are used for transplantation to treat hematological cancers and some solid tumors, following first line treatment with chemo- and radiotherapy in order to reduce tumor burden and achieve long term remission⁴. As drug resistance and relapse remain major problems, autologous and human leukocyte antigen (HLA)-matched allogeneic HSCT are used as potentially curative cell therapy treatment for malignant and non-malignant hematological diseases. In allogeneic HSCT, donor T cells mediate a powerful graft-versus-tumor (GVT) effect⁵. However, T cells can also cause GVHD and therefore limit the overall effectiveness of allogeneic HSCT. Various methods of T cell depletion reduce the risk of GVHD and allow in addition transplantation across the histocompatibility barrier, but might increase the risk of graft rejection or relapse. Natural Killer (NK) cells have been described to eliminate leukemia relapse and graft rejection and to protect patients against GVHD in a haploidentical HSCT setting⁶. Haploidentical NK cells in a stem cell transplantation setting have shown to reduce GVHD without causing GVHD by themselves⁷. This is mainly by their ability to inhibit and lyse GVHD inducing T cells and host antigen presenting cells (APCs), which are critical for the activation of donor T cells in GVHD induction. Furthermore, there is clinical evidence, that high NK cell doses in haploidentical unrelated HSCT prevent severe GVHD, while preserving the GvT effect⁸.

NK cells are the third major subpopulation of lymphocytes, beside CD3+ T-cells and CD19+ B-cells. NK cells are important effector cells of the innate immune system because they can exert rapid effector function without prior sensitization, i.e. “Natural” killing. Therefore, NK cells play a key role in early defense against viral and bacterial infections and in tumor immune surveillance. NK cells are present in lymphoid organs and various non-lymphoid tissues. Beside their cytolytic activity, NK cells are able to produce a wide variety of cytokines and chemokines to influence the other cellular compartments of the immune system. NK cells can be defined as CD56 positive CD3 negative lymphocytes comprising 5-15% of the circulating lymphocyte population. They are subdivided into two major subsets based on their CD56 expression levels. CD56^{dim} NK cells, accounting for approximately 90% of peripheral blood NK cells have marked direct cytolytic potential using granzyme and perforin mediated killing and express high levels of the low affinity Fc receptor III (FcRγIII; recognized by CD16) allowing them to mediate antibody-dependent cellular cytotoxicity (ADCC) In contrast, CD56^{bright} NK cells, representing ˜10% of all NK cells, have predominantly immune regulatory functions mediated by a potent production of cytokines, without exerting direct cytolytic function.

NK cells recognize and kill infected or malignant-transformed cells through signals from germ line-encoded inhibitory receptors (IR) or activating receptors (AR). The combination of these signals balances and modulates NK cell effector functions.

The activating signals are mediated by ARs of which the most important receptors, beside CD16 described above, are CD314, CD226 and the natural cytotoxicity receptors CD334, CD335 and CD336. Cytolytic NK cells can induce tumor cell death without prior immunization as well as produce cytokines such as IFN-γ TNF-α and GM-CSF that are key mediators in activating dendritic cells in lymph nodes thereby linking innate NK cell-based immunity to adaptive T cell-mediated immunity.

In order to boost patients' own immune effector cells such as autologous T and NK cells, trials assessing the effects of IL-2 administration on activation and expansion of autologous NK cells in patients with cancer have been performed^{9,10}. However, results have been variable and the outcome is highly dependent on the type of tumor and doses of the IL-2 treatment. Furthermore, high-dose IL-2 treatment is associated with life-threatening toxicities, represented by capillary leak syndrome and pulmonary edema^{11,12}. IL-15 may be more efficient than IL-2 to expand autologous or haploidentical NK cells because it promotes their survival, however IL-15 has just entered phase I/II clinical trials (NCT01021059, NCT01369888, NCT01385423, NCT01572493) and the dosage and effect on autologous or haploidentical NK cells or other immune cells has not been described in humans up to date^{13,14}. Beside the activation of autologous or haploidentical NK cell cytotoxicity using cytokines, several other strategies to boost autologous NK cell mediated tumor killing have been postulated as combinatorial therapies, such as the use of small molecules or antibodies. Monoclonal antibodies like rituximab (anti-CD20) have been used in patients with non-Hodgkin's lymphoma to activate NK cell's ADCC effector function^{15,16}. Nowadays also some drugs like Thalidomide, Lenalidomide, Bortezomib and Imatinib are used to boost the immune response by boosting autologous or haploidentical NK cell survival, proliferation and activation in vivo¹⁷⁻¹⁹. Some more complex mechanisms for autologous or haploidentical NK cell activation have emerged by using specific vaccines acting on toll-like receptors, which activate autologous or haploidentical NK cells directly or indirectly by influencing dendritic cells (reviewed in²⁰).

So far, early studies using autologous NK cell infusions were not able to show a significant clinical benefit. But recent clinical trials in both the transplant and non-transplant setting have clearly demonstrated that allogeneic haploidentical NK cell reactivity can induce clinical remission in AML patients. In the setting of HLA-mismatched, haploidentical allogeneic SCT, it has been demonstrated that NK cell alloreactivity can control relapse of AML without causing severe GVHD. Based on the encouraging clinical results in allogeneic haploidentical SCT, adoptive transfer of haploidentical NK cells have been used to induce anti-cancer immunity in AML and other malignancies. In order to study the role of haploidentical NK cells as a potential curative treatment, direct infusions of haploidentical NK cells represent a possible approach to enhance antitumor immunity in cancer patients. But also in the non-transplant setting it has been demonstrated that allogeneic haploidentical NK cell infusions can induce hematologic CR in poor-prognosis elderly AML patients. Similar treatment options have been successfully explored in childhood AML for inducing long-term remission. A combination of chemotherapy and haploidentical NK cell infusion was associated with limited non-hematologic toxicity and no induction of GVHD. However, recently it has been reported, that patients receiving haploidentical NK cells for immunotherapy developed severe GVHD²¹.

The first successful transfer of haploidentical NK cell in a non-transplant setting was demonstrated by the study of Miller and colleagues²². They demonstrated that allogeneic haploidentical NK cell infusions up to 2×10⁷ cells/kg body weight were well tolerated, without the evidence of induction of GVHD. In this study, a heterogeneous group of 43 patients with advanced cancers (melanoma, renal cell carcinoma and AML) received haploidentical NK cell infusions enriched from healthy donor aphaeresis products together with IL-2 in a non-transplantation setting. AML patients received intensive immunosuppressive conditioning chemotherapy prior to haploidentical NK cell infusions, to prevent immunologic rejection of infused donor cells and to induce survival factors (e.g. IL-15) or to deplete cellular and soluble inhibitory factors. The high dose cyclophosphamide and fludarabine (Hi-Cy/Flu) regimen mediated prolonged in vivo persistence and expansion of infused haploidentical NK cells. Interestingly, 5 out of the 19 AML patients obtained CR after adoptive transfer of enriched NK cell product, but it remains to be proven whether solely the haploidentical NK cells were responsible for the clinical effect since the infusion products contained a mean of 40%±20% CD56+CD3− haploidentical NK cells (range 18%-68%), 19±2% B cells, 25±1.6% monocytes and around 1% CD3+ T cells. Although T cell administration was limited to 2.1±0.3 (range 0.5-6.5)×10⁵ T cells/kg, alloreactive T cell responses may have played some role in the observed graft versus leukemia (GVL) effect. Toxicity was limited to constitutional symptoms including low-grade fever, chills and myalgia mostly due to low-dose IL-2 injections post haploidentical NK cell infusion. These findings suggest that haploidentical NK cells can persist and expand in vivo (>1% engraftment at day 7 and beyond) and potentially reduce relapse in AML.

A more recent study (“NKAML” study) by Rubnitz and coworkers, reported the treatment of pediatric AML patients from 0.7-21 years of age in first complete remission (CR) with haploidentical NK cell infusions. In this “NKAML” study a median of 2.9*10⁷ haploidentical NK cells/kg body weight were infused and additionally 6 subsequent doses of 1×10 IU/m² IL-2 were given. Haploidentical NK cell engraftment has been detected for a median of 10 days with a significant expansion of KIR-HLA mismatched haploidentical NK cells.

Finally, Curti et al. reported the successful transfer of haploidentical NK cells in 13 elderly AML patients, from which 5 had active disease, 2 were in molecular relapse and 6 were in morphological CR. Curti et al. infused a median of 2.74×10⁶ haploidentical NK cells/kg with a T cell content under 10⁵/kg. Most interestingly, 1 of the 5 patients with active disease reached transient CR and the 2 patients in molecular relapse achieved CR lasting for 4-9 months. Furthermore, 3 of the 6 patients in CR remained disease free after 18-34 months. Infused haploidentical NK cells were found in peripheral blood and bone marrow and they showed alloreactivity against recipient's leukemia target cells in in vitro studies. Together, these three studies underline the feasibility of using haploidentical NK cell infusion in a non-transplant setting with limited GVHD. However, the haploidentical NK cell products used in these studies were limited in cell numbers as generally not more than 1×10⁷ haploidentical NK cells/kg bodyweight were administered as a single infusion in adult patients. Additionally the products still contain allogeneic T cells, which indicate a certain risk to develop GVHD. Therefore, to increase the clinical application of cellular adoptive immunotherapy, GMP-compliant isolation, activation and ex vivo expansion procedures are needed to provide optimal cell products with higher cell number, purity and functional activity.

Nowadays, innovative approaches in cellular therapy turn away from haploidentical matching principle and use autologous cells such as T cells with genetic modifications (chimeric antigen receptor T cells; CAR-T)²³⁻²⁸. However, such approaches will require difficult logistics and end up in high costs for applying individual treatments for patients.

As described above, autologous or haploidentical adoptive cell transfers clearly have drawbacks, such as low cell numbers and/or low activity of the desired cells, uncertain availability of infusion product at the day of transfer, the presence of contaminating undesired cells hi the infusion product, high dose immunosuppressive conditioning before transfusion and the necessity to generate cells for adoptive transfer on an individual base, because of the autologous or haploidentical nature of the adoptive transfer. The present invention solves at least one of these draw backs by using a novel approach to immunotherapy, which does not need haploidentical matching criteria and enables the production and storage of large amounts of immune effector cells that can be used off-the-shelf for adoptive cell immunotherapy.

Solid tumors in breast, colon, rectum, lung, prostate, cervix and ovaries upon diagnosis are treated by conventional methods (surgery, chemo and radiotherapy) to reduce tumor load. However, these tumors develop resistance to chemotherapy, often metastasize, spreading to lymph nodes and adjacent organs with increased number of circulating tumor cells in peripheral blood²⁹.

Cervical cancer is one of the challenging disease to treat in advanced conditions. Persistent infection of the cervical epithelium by high-risk human papilloma virus (HPV) can lead to cervical intraepithelial neoplasia which may progress to invasive cervical cancer, such as squamous cell carcinoma, adenosquamous cell carcinoma or adenocarcinoma^{30,31}. Treatment for cervical cancer includes conventional surgery, chemotherapy and/or radiation. In addition, in advanced (metastatic) disease, targeted therapies are widely explored. Unfortunately, targeted intervention strategies using small molecules, angiogenesis inhibitors and monoclonal antibodies directed against specific tumor antigens and proliferation pathways have had limited success in restricting cervical tumor growth so far^{32,33}.

In cervical cancer, epidermal growth factor receptor (EGFR) is variably expressed in 80% of the tumor tissues³⁴. Overexpression of EGFR has been associated with poor prognosis in cervical cancer, making EGFR an obvious candidate for therapeutic targeting^{35,36}. Treatment with cetuximab (chimeric IgG₁, anti-EGFR mAb) as monotherapy or cetuximab in combination with chemotherapy was ineffective in patients with cervical cancer, in spite of the apparent absence of activating mutations in KRAS (Kirsten rat sarcoma viral oncogene) in the EGFR pathways³⁷. However, as previously published, combination of NK cells and cetuximab could lead to improved killing in EGFR expressing colon cancer, so this can be studied in cervical cancer as well, enabling improvement of anti-EGFR mAb therapy, besides increased killing of cervical tumors by NK cells³⁸. Further, it has been reported that Indoleamine 2,3 dioxygenase (IDO) overexpression on tumor cells prevents immune cells from recognizing tumor cells³⁹.

Infiltrating NK cells are observed in low-grade and high-grade cervical intraepithelial neoplasia lesions and to a lesser extent in cervical carcinoma^{40,41}. In vitro studies have shown that peripheral blood NK cells (PBNK) are able to kill HPV-infected cell lines⁴². However, NK cells are often dysfunctional and low in number in cervical cancer patients, and thereby unable to mount efficient cytotoxicity against tumors^{43,44}. NK cytotoxic function is also counteracted by several cervical tumor escape mechanisms, including low expression of activating NK cell receptor ligands (e.g. MICA/B, ULBPs, Nectin, PVR) and aberrant expression of suppressive non-classical HLA molecules (e.g. HLA-E and -G) by tumor cells^{42,45,46}. Ex vivo expanded autologous NK cells, adoptively transferred for the treatment of solid tumors, in most studies have yielded disappointing results, underscoring the dire need for the development of more powerful therapeutic approaches to overcome tumor-associated NK cell dysfunctionality and the inherent resistance to cytolysis of cancer cells. Immunotherapy of cervical cancer has been clinically explored with limited success. Efforts so far have mostly focused on vaccination approaches against HPV-derived oncogenes (E6 and E7) to trigger an efficacious antitumor T-cell response⁴⁷. Failure to improve clinical outcome may at least in part be due to extensive HLA down-regulation commonly observed in cervical cancer. The fact that cervical tumors often show downregulation in MHC Class-I expression, often unresponsive to T cells, but highly favors lysis by NK cells.

Colorectal cancer is another challenging disease to treat in advanced conditions. Colorectal cancer (CRC) is the fourth leading cause of cancer related deaths in the world. Distant metastasis is a common threat occurring in more than half of the CRC patients, mainly in the liver, followed by lungs⁴⁸⁻⁵⁰. In advanced and metastatic conditions, epidermal growth factor receptor (EGER) targeted therapies are approved for use either in combination with chemotherapy or in chemo refractory conditions for EGFR⁺ RAS^{wt} CRC patients, Anti-EGFR monoclonal antibodies (mAbs) Cetuximab, IgG₁ (Erbitux®) and Panitumumab, IgG₂ (Vectibux®) are currently in use⁵¹. However, these drugs are ineffective in CRC patients who have mutations in RAS gene, thus leaving 42% of the metastatic CRC (mCRC) population with no standard treatment option⁵², Hence, there is unmet clinical need for refractory cancers and therefore greater emphasis has been placed on developing active therapeutic approaches like RAS-MAP kinase pathway inhibitors and combination of chimeric monocloncal antibodies (mAbs) to overcome tumor cell resistance to therapeutic drugs⁵³⁻⁵⁵.

Several factors influence the outcome of prognosis in CRC, the role of immune cells in controlling tumor cannot be ignored⁵⁶. Clinical studies, as reviewed, aimed to restore immune system function, either by eliciting immune response or by recruiting immune cells to tumor sites are under investigation⁵⁷. Among cellular therapies, T cell based therapies involving adoptive transfer of ex vivo expanded T cells, use of check point inhibitors and chimeric antigen receptor specific T cells (CAR-T) are more commonly used now in the clinics⁵⁸. However, mCRC patients treated with developed severe side effects, questioning the safety of genetically modified T cells in CRC^{59,60}.

Natural killer (NK) cells could be a viable option under these circumstance to target CRC tumors. NK cells can act without prior sensitization, spontaneously identifying and eliminating tumors or infected cells under expressing major histocompatibility complex (MHC) class I⁶¹. Severely diminished or aberrant expression of MHC class I reported in majority of colorectal carcinomas⁶², often unresponsive to cytotoxic T cells, and makes them an ideal target for NK mediated lysis. NK cells, part of innate immune system is identified by the expression of CD56, characterized into two subsets based on CD16, a low affinity FcγRIIIa receptor. The majority of NK cells are CD56^{dim}CD16⁺, plays an active role in NK cell cytotoxicity and engages with IgG₁ therapeutic monoclonal antibodies (mAbs) Ike cetuximab via CD16 to perform antibody dependent cell mediated cytotoxicity (ADCC), whereas CD56^{bright}CD16⁻ NK cells are mainly immune regulatory in function secreting cytokines and are less cytotoxic than CD56^{dim} cells⁶³.

In most cases in CRC patients, the low frequency and dysfunctional nature of NK cells, together with immunosuppressive tumor microenvironment, highly affected its functionality and active migration to the tumor site⁶⁴, Hence, various methods to augment NK cell function using cytokines or therapeutic ADCC enhancing mAbs are being extrapolated to increase NK cell numbers in peripheral blood and its propagation into blood vessels supplying the tumor²⁰. Another alternative is to adoptively transfer ex vivo manipulated and expanded autologous or allogeneic NK cells. Autologous NK cells so far have failed to demonstrate significant therapeutic benefits in solid tumors^{66,87}. Lack of anti-tumor effects from autologous NK cells, shifted the focus towards developing allogeneic NK cells as a potential adoptive cell therapy for the treatment of solid tumors. We demonstrated from our previous studies, that, allogeneic PBNK cells in combination with cetuximab can effectively target RAS mutant CRC tumors⁶⁸. Further, allogeneic NK cells unlike T cells do not induce graft versus host disease (GVHD), thereby considerably reducing treatment related toxicities²².

In these cases, NK cell-based therapies may prove more effective than T-cell-based approaches. Indeed, the role of the innate immune response in host defense and viral clearance during (early) infection is well recognized⁶⁹. NK cells are potent in exerting rapid cytotoxicity by releasing cytotoxic granzyme B and perforin in order to lyse virus-infected cells and tumor cell targets, NK cell-mediated cytolysis of tumor cells may be enhanced by binding to tumor-targeted IgG1 monoclonal antibodies, resulting in antibody dependent cell mediated cytotoxicity (ADCC) Alternatively, cytokine-activated allogeneic NK cells from healthy donors may be used for adoptive cell transfer⁷⁰.

Clinical studies where application of allogeneic related or haplo-identical PBNK cells were used to treat renal cell carcinoma, metastatic melanoma, breast and ovarian cancer have often failed to demonstrate significant therapeutic benefits^{22,71}. The majority of NK cell products derived from peripheral blood mononuclear cells are feeder cell based cultures, which are severely limited by their purity, ability to expand in vivo and were often unable to exert adequate cytotoxicity against tumors, besides they do not have sufficient numbers for multiple doses of NK cell infusions⁷⁰. However, an alternative would be to use umbilical cord blood CD34+ derived NK cells, which are feeder cell free cultures, can be efficiently expanded up to 10,000 fold, maintaining high purity (92%±2%; n=4), with undetectable CD3+ or CD19+ cells, and demonstrates cytotoxicity against tumor cells^{72,73}.

Another alternative is to adoptively transfer ex vivo manipulated and expanded autologous or allogeneic NK cells, Autologous NK cells so far have failed to demonstrate significant therapeutic benefits in solid tumors⁶⁵⁻⁶⁷. Lack of anti-tumor effects from autologous NK cells, shifted the focus towards developing allogeneic NK cells as a potential adoptive cell therapy for the treatment of solid tumors. However, very few data exist on the clinical efficacy of NK cells in eradicating solid tumors.

### SUMMARY OF THE INVENTION

In a first embodiment, the invention provides a composition comprising an immune effector cell, for use in a non-autologous immunotherapy, wherein the composition is to be administered to an individual, characterized in that the immune effector cell is non-haploidentical with respect to the individual.

As stated in the introductory part, up to the present invention, immunotherapy has been performed using autologous or allogeneic, haploidentical adoptive cell transfer, e.g. in a hematopoietic stem cell (HSC) transplantation or with more or less purified immune cell subsets. Up to the present invention, it was thought that for adoptive immune effector cell transfer, only partial mismatch, i.e. the donor and recipient must be at least haploidentical, is allowed for safety reasons. Donors, therefore, are sought within the family blood line (child—parent, siblings, aunt/uncle—niece/nephew, etc.). Using a composition as defined by the invention for use in immunotherapy, however, the inventors have shown that immune effector cell adoptive transfer beyond the classical haploidentical mismatch is safe and efficacious.

With the term “immune effector cell” as used herein is meant: A cell of the myeloid or lymphoid lineage, which exerts an immunologic function either by release of a immunologic active substance, which could have an direct or indirect effect towards an immunologic relevant target or whereas it exerts a direct cytotoxic effect based on a stimulation by the immunologic relevant target. Preferably, the term immune effector cell is reserved for those cells that, similar to a T-lymphocyte or a natural killer cell, is activated by receiving at least one activation signal from a target cell, preferably a tumor cell, and upon activation exerts a direct cytotoxic effect towards this target cell.

With the term “non-autologous” is meant that in a transfusion or transplantation setting, the donor and the recipient is not the same individual, i.e. not autologous. The word autologous is Greek in origin. The definition is exact ‘autos’ means self and ‘logus’ means relation. Thus, the meaning is ‘related to self’. Autologous blood transfusion, for instance, designates the reinfusion of blood or blood components to the same individual from whom they were taken. Non-autologous, as used herein thus means the infusion of cells derived from one individual to another individual. Preferably, the donor individual and the recipient individual are not related by blood, i.e. they are not siblings, parent and child, uncle or aunt and niece or nephew, cousins, etc.

The term “immunotherapy” denotes a treatment that uses certain parts of a person's immune system to fight diseases such as cancer. The parts of the immune system can be either from the person having the disease, but also from another person, called “donor”, such as the case in the present invention. A composition for use according to the invention is preferably used in cell-based immunotherapy, wherein immune effector cells, derived from an autologous, non-haploidentical donor are administered to a recipient in need thereof.

A general definition of “haploidentical” is “sharing a haplotype; having the same alleles at a set of closely linked genes on one chromosome”. With regard to haploidentical in relation to HLA, this means that the donor and recipient have the same set of closely linked HLA genes on one of the two Number 6 chromosomes they inherited from their parents. Rather than being a perfect match for each other, a haploidentical donor and recipient are a half-match.

Parents are always a half-match for their children and vice versa. Siblings have a 50 percent chance of being a half-match for each other. (They have a 25 percent chance of being a perfect match and a 25 percent chance of not matching at all).

The gene loci for major HLA molecules show genetic variation in more than 8,500 different alleles for MHC class I genes and more than 2,500 alleles for MHC class II genes.

A haplotype, therefore consists of the full HLA-gene phenotype for every HLA-locus and its allele. The allelic combinations of those would be already more than 21 million. The likelihood of finding a haploidentical (or better) unrelated match is therefore very, very small.

The term “non-haploidentical” as used herein thus denotes a HLA mismatch beyond the classical haploidentical mismatch. Preferably the term “non-haploidentical” is used herein for the situation wherein the donor of the immune effector cell and the recipient of the immune effector cell do not share at least one set of closely linked HLA genes on one of the two Number 6 chromosomes. In other words, this means that at least one of the HLA molecules HLA-A, HLA-B, HLA-C, HLA-DR, HLA-DP, or HLA-DQ does not have at least one allele in common between the immune effector cell of the invention and the recipient of the immune effector cell, i.e. the individual receiving the immunotherapy. Preferably the HLA molecule that does not have at least one allele in common between immune effector cell of the invention and recipient is one of HLA-A, HLA-B or HLA-C. More preferably, at least two of HLA-A, HLA-B or HLA-C or, most preferably, all three do not have at least one allele in common between the immune effector cell of the invention and the recipient. As said above, typically and in the majority of cases, HLA is mismatched beyond haploidentical if the donor and recipient are not related by blood. With the term “mismatched beyond haploidentical” or “non-haploidentical” is thus meant that there is less match between the donor and the recipient than there would be if the two were haploidentical.

This invention preferably uses cells that are generated with a GMP-compliant culture system for the generation of large batches of immune effector cells, e.g. from umbilical cord blood (UCB)-derived CD34+ progenitor cells, preferably without T cell contamination. It is advantageous to use such cells as they have higher conformity, making, e.g., regulatory processes much easier. At the same time, the present invention enables usage of such large batches of immune effector cells, because previously, individual batches had to be generated, based on the at least partial match with the envisaged recipient because of safety concerns. The present invention, however, shows that immune effector cells as defined by the invention, mismatched beyond being haploidentical are safe to use in immunotherapy and that they show efficacy.

Preferably, a composition for use according to the invention further comprises at least one excipient, such as for instance water for infusion, physiologic salt solution (0.9% NaCl), or a cell buffer, preferably consisting of a physiologic salt solution substituted with a protein component such as human serum albumin (HAS).

In order for a composition of the invention to be used in such non-haploidentical mismatched situation, the inventors have found out that it is preferred that the immune effector cell is not a B-cell or a T-cell (i.e. CD3 and CD19 negative), but that it is positive for Neural Cell Adhesion Molecule (NCAM). Such cell has cytolytic activity, without reacting vigorously with ubiquitous present HLA-expressing cells of the recipient. The latter is also known as a Graft versus Host (GvH) reaction, which can be life threatening. The use of immune effector cells for immunotherapy according to the invention did not result in GvH related symptoms in any of the patients tested. In a preferred embodiment, a composition for use according to the invention does not result in graft versus host disease.

In a preferred embodiment, a composition for use according to the invention is provided, wherein the immune effector cell is positive for Neural Cell Adhesion Molecule (NCAM) and negative for CD3 and CD19.

Neural cell adhesion molecule (NCAM), is a glycoprotein of Immunoglobulin (Ig) superfamily expressed on the surface of neurons, glia, skeletal muscle and natural killer cells. NCAM, also called CD56, has been implicated as having a role in cell-cell adhesion, neurite outgrowth, synaptic plasticity, and learning and memory. NCAM is preferably used to define the population of differentiated immune effector cells for use according to the invention and can be used to discriminate the infused effector cells from patient's natural killer cells in the peripheral blood.

CD3 is part of the T cell receptor (TCR) complex, which is a molecule to be found on the surface of only T lymphocytes (or T cells). CD3 is also called the T cell co-receptor. The TCR complex is responsible for recognizing antigens, represented by small peptides binding to major histocompatibility complex (MHC) molecules. CD3 is found bound to the membranes of all mature T-cells, and in virtually no other cell type, This high specificity, combined with the presence of CD3 at all stages of T-cell development, makes it a useful to identify T-cells in tissue sections. CD3 is involved to recognize and reject foreign HLA is thus related to GVHD.

CD19 is part of the B cell receptor complex, which is present throughout the whole lifespan of B cells. B cells, also known as B lymphocytes, are a white blood cell subtype. Their function as immune effector cells as being a component of the adaptive immune system by secreting antibodies and they also can present antigen.

As B cells are potentially infected with Epstein-Barr virus (EBV), which has the potential to develop an EBV induced lymphoma, the invention aim to protect patients from such risks by defining the product CD19 negative.

The inventors have further found out that it is advantageous that the immune cell of the invention expresses one or more of CD159a, CD314, CD335, CD336 or CD337.

CD159a and CD85j/Leukocyte Ig-like receptor-1 (LIR-1) are inhibitory receptors expressed on cytotoxic immune effector cells such as CD8 positive T cells and natural killer (NK) cells. They are known to bind to HLA-E and HLA-G respectively, therefore preventing cytotoxic cells from attacking normal (healthy) tissues, which normally express HLA-E and/or HLA-G^{74,75}. This is a very efficient mechanism to prevent the immune effector cells used in this invention from attacking normal tissues, as the immune effector cells are used in a mismatched setting, beyond being a haploidentical mismatch.

CD314 is a C-type lectin-like protein known to be expressed on CD8+ T cells, γ/δ T cells, and NK cells. CD314 binds to MHC class-1 chain-related protein A (MICA), MICB, and UL16-binding proteins (ULBPs) activates cells by non-covalent association with DAP10 or DAP12 adaptors. CD314 is a costimulatory receptor for TCR-mediated T cell proliferation and cytokine production and in addition a primary activation receptor on NK cells. The interaction of CD314 with its ligands shows important responses against pathogen and tumor cells, and in the pathogenesis of autoimmune diseases.

CD335 as member of the natural cytotoxicity receptor (NCR) family which triggers cytotoxicity in for instance NK cells. CD335 is directly involved in target cell recognition and lysis, and is for instance expressed on CD3−CD56+ NK cells.

CD336 is a type I transmembrane protein, member of the natural cytotoxicity receptor family that is expressed a subset of γ/δ T cells and on IL-2 activated NK cells. CD336 enhances for instance NK cell mediated cytolysis of virus infected cells and tumor cells.

CD337 is a type I transmembrane protein, member of the natural cytotoxicity receptor family and is for instance expressed on resting and activated NK cells. NKp30 enhances for instance NK cell cytolysis of tumor cells that are deficient in MHC class I molecules.

It is thus preferred to have at least one, preferably two, more preferably at least three, most preferably all four of the above mentioned cell surface molecules expressed in an immune effector cell present in a composition for use according to the invention.

In a preferred embodiment, a composition for use according to the invention is provided, wherein the immune effector cell has cytolytic activity towards a tumor cell and/or a virus infected cell, preferably a tumor cell.

In a preferred embodiment, a composition for use according to the invention is provided, wherein the immune effector cell expresses one or more of the following cell surface markers: CD159a, CD314, CD335, CD336, and CD337. Preferably, the immune effector cell expresses at least CD314, CD336, or both. As the interaction of CD314 with its ligands shows important responses to tumor cells and also CD336 enhances T cell as well as NK cell mediated cytolysis of tumor cells, this combination on the immune effector cells as defined by the invention is very useful for the immune effector cells to get activated by and kill a tumor cell.

Typically, the composition of the invention comprises a plurality of cells. It is not necessary for all the cells in the composition to have the features and effects as defined by the invention. However, it is preferred to have at least a certain percentage of immune effector cells as defined in the invention in the composition for use according to the invention in order to have the right balance with regard to efficiency (during production) and efficacy (in the clinics). In a preferred embodiment, a composition for use according to invention is provided, wherein the composition comprises a plurality of cells, characterized in that 30-100%, preferably 30-90%, more preferably 30-80%, more preferably 30-70%, more preferably 30-60%, more preferably 30-50%, most preferably 30-40% of the plurality of cells is an immune effector cell as defined by the invention. Preferably, the composition comprising a plurality of cells is characterized in that 40%-100%, more preferably 50-100%, more preferably 60-100%, more preferably 70-100%, more preferably 80-100%, most preferably 90-100% of the plurality of cells is an immune effector cell as defined by the invention. Other preferred ranges of immune effector cells as defined by the invention within a composition for use according to the invention are: 40-90%, 50-90%, 60-90%, 70-90%, 80-90%, 40-80%, 50-80%, 60-80%, 40-70%, 40-60%, 50-60% or 40-50%. For production efficiency, a lower percentage of the immune effector cells as defined by the invention is desired, whereas on the other hand for clinical efficacy and for regulatory reasons a higher percentage of the immune effector cells as defined by the invention is desired.

There are several ways, which are known by the skilled person, to generate immune effector cells that are NCAM positive and CD3 and CD19 negative. Such immune effector cells can for instance be generated ex vivo from a stem cell or progenitor cell, in particular from a stem or progenitor cell that is CD34 positive. CD34 is a cell surface glycoprotein and functions as a cell-cell adhesion factor and mediates the interaction of stem cells to bone marrow extracellular matrix or directly to stromal cells. CD34 is expressed on multipotent hematopoietic stem cells and also on lineage specific hematopoietic progenitor cells. CD34 is clinically used for the definition of the quality of stem cell transplant by describing the content of stem and progenitor cells responsible for the engraftment of a new immune system.

In a preferred embodiment, the invention provides a composition for use according to the invention, wherein the immune effector cell is generated ex vivo from a stem cell or from a progenitor cell, wherein the stem cell is preferably a CD34+ stem cell and/or the progenitor cell is preferably a CD34+ progenitor cell.

It is in particular preferred, from a regulatory perspective, but also from a perspective of efficiency, that a composition for use according to the invention is obtained from a single donor. Even more preferred is that a single donor provides more than one treatment dose, such that large scale batches can be produced, be cleared or certified, and used off-the-shelf at the moment a random individual must be treated with a composition for use according to the invention. Preferably the generation of immune effector cells suffices for at least 10, more preferably at least 20, more preferably at least 50, more preferably at least 100, most preferably at least 200 or more single treatment doses for use according to the invention. If e.g. about 5×10⁸-1×10¹⁰ cells are to be used for a single treatment, it is preferred that for treating, e.g. 10 individuals at least 10¹¹ immune effector cells are generated from the CD34 positive stem or progenitor cells from one single donor. The thus generated large batch of cell can be easily transferred to vials with the correct amount of cells (e.g. about 5×10⁸-1×10¹⁰) cells per vial, frozen and stored. In the moment a composition for use according to the invention is needed, one of such vials can be thawed and prepared for administration to the individual in need of immunotherapy. In a preferred embodiment, a composition for a use according to invention is provided, wherein the plurality of cells are derived from cells obtained from a single donor. Preferably, the plurality of cells are derived from at least one of umbilical cord blood and bone marrow, as these are rich sources of CD34 positive stem and/or progenitor cells.

Because of the possibility to use off-the-shelf compositions comprising immune effector cells in a setting that does not require partial matching, as defined by the invention, the composition for use according to the invention shifts cell adoptive therapy a step further from personalized medicine towards more generic medication as it is no longer necessary to search for individual donors to match individual recipient. This also has a beneficial impact on the costs of treatment.

With “off-the-shelf” as used herein is meant that such composition is prepared and stored for direct usage when needed. In particular a composition that is available “off-the-shelf” is not generated for one specific recipient but in general can be used for different recipients at different time points. The composition as defined by the invention can for instance be frozen and, when needed, thawed and used as defined by the invention. A composition as defined by the invention enables large scale production of GMP generated immune effector cells that can theoretically be provided within minutes when needed for any random recipient.

The invention preferably uses a composition that is the result of an efficient expansion and differentiation cell culture process to generate functional immune effector cells from UCB CD34+ stem and progenitor cells⁷⁶. Such composition preferably contains NCAM positive, CD3 negative effector cell subsets that uniformly express high levels of activating receptors, while they differentially express inhibitory receptors such as the receptor complex CD94/ECG2A and killer-cell immunoglobulin-like receptors (KIRs). Within the current invention the selection of donor and recipient is preferably not matched for a mismatch between the recipients HLA related KR ligand and the KIR genotype of the donor. Moreover, a composition for use according to the invention mediates strong cytolytic activity against tumor cells, such as for instance AML cells ex vivo that can be correlated with granzyme B degranulation and IFNγ release upon target cell engagement (data not shown).

In order to utilize ex vivo-expanded immune effector cells as defined by the invention for adoptive immunotherapy in poor-prognosis AML patients, the method was adapted into a closed-system bioprocess for production of allogeneic immune effector cell batches under GMP conditions⁷². The developed immune effector cell generation procedure consists of two culture steps. The first step involves the expansion of CD34+ cell progenitors in 14 days of culture. The second step consists of the differentiation of the expanded progenitor cells into the immune effector cell lineage, which requires an additional 4-week culture period. Systematic refinement of the system, using the proprietary GMP-compatible serum-free Glycostem Basal Growth Medium (GBGM), resulted in a clinical applicable protocol enabling the ex vivo expansion and differentiation of CD34+ cells from frozen umbilical cord blood (UCB) units to more than a 15,000-fold expansion into NCAM positive and CD3 negative immune effector cells with very high purity⁷⁶.

Large-scale experiments using WAVE Bioreactor™ system (GE Healthcare) demonstrated that the two-step expansion and differentiation protocol reproducibly generates between 1-10×10⁹ NCAM positive immune effector cells from UCB-derived CD34+ cells enriched by the CliniMACS cell separator (Miltenyi Biotec) with a high purity. T and B cells were not detectable by flowcytometry (<0.01% CD3+ cells and <0.01% CD19+ cells, respectively).

In one preferred embodiment, a composition for use according to the invention is provided, wherein the composition is generated ex vivo in a process comprising the steps of:
a) obtaining a sample comprising CD34+ hematopoietic stem and/or progenitor cells
b) affinity purification of CD34+ hematopoietic stem and/or progenitor cells from the sample obtained in a);
c) expanding the purified CD34+ hematopoietic stem and/or progenitor cells obtained in b) in a basal growth medium supplemented with human serum, a low-dose cytokine cocktail consisting of three or more GM-CSF, G-CSF, LIF, MIP-1α and IL-6, a combination of two or more of high-dose cytokines including SCF, Flt3L, IL-7 and TPO and a low-molecular weight heparin; and,
d) differentiating the expanded CD34+ hematopoietic stem and/or progenitor cells obtained in c) in a basal growth medium supplemented with human serum and IL-15 and additional one or more cytokines including SCF, Flt3L, IL-7, IL-12, IL-18 and IL-2,
e) harvesting the cells generated in d) and generating a composition as defined the invention.

A sample comprising hematopoietic stem and/or progenitor cells may be obtained in any possible way, such as for instance obtain or collect a stem and/or progenitor containing cell source, such as bone marrow, cord blood, placental material, peripheral blood, peripheral blood of a person treated with stem cell mobilizing agents, generated ex vivo from embryonic stem cells or any deviates thereof using cell culturing steps or generated ex vivo from induced pluripotent stem cells and any deviates thereof using cell culturing steps. Hematopoietic stem and/or progenitor cells can be further purified from such stem and/or progenitor containing cell sources using affinity purification methods.

With the term “ex vivo” is meant that the process or method performed is not used within a living individual, but for instance in a device able to culture cells, preferably an open or a closed cell culture device, such as a culture flask, a disposable bag or a bioreactor.

In a preferred embodiment, a composition for use according to the invention is provided, wherein the composition is generated ex vivo as described above, wherein in step d, the additional at least one or more cytokine is SCF, preferably SCF and IL-2, more preferably SCF, IL-2 and IL-7, more preferably SCF, IL-7, IL-2 and IL-12 and most preferably SCF, IL-7, IL-2, IL-12 and IL-18.

In another preferred embodiment, a composition for use according to the invention is provided, wherein the composition is generated as described above, wherein in step d, the additional at least one or more cytokine is SCF, preferably SCF and Flt3L, more preferably SCF, Flt3L and IL-2, more preferably SCF, Flt3L, IL-2 and IL-7, more preferably SCF, Flt3L, IL-7, IL-2 and IL-12 and most preferably SCF, Flt3L, IL-7, IL-2, IL-12 and IL-18.

In a preferred embodiment, a composition for use according to the invention is provided, wherein the composition is generated as described above, wherein in step c, the combination of at least two cytokines are TPO and Flt3L, more preferably SCF and Flt3L, more preferably SCF and TPO, and most preferably SCF and IL-7.

In another preferred embodiment, a composition for use according to the invention is provided, wherein the composition is generated as described above, wherein in step c, the combination of at least two or more cytokines are SCF and Flt3L, more preferably SCF, Flt3L and TPO, more preferably SCF, Flt3L and IL-7, more preferably SCF, TPO and IL-7, and most preferably SCF, TPO, Flt3L and IL-7.

With the term “CD34+ stem cell” is meant a multipotent stem cell, which expresses the CD34 antigen on the cell surface, preferably being a stem cell, which is able to develop in all certain types of blood cells and more preferably a cell, which can give rise to lineage specific progenitor cells of the blood lineages.

With the term “CD34+ progenitor cell” is meant a multipotent progenitor cell, which expresses the CD34 antigen on the cell surface, preferably being a progenitor cell, which is able to develop in various types of blood cells and more preferably a cell, which can give rise to lineage specific progenitor cells of the certain blood lineages.

With the term “affinity purification” as used herein is meant, that the cells to be purified are labelled, by targeting for instance a specific epitope of interest for separation purposes, for instance targeting an antigen with an antibody coupled to an agent suitable for detection by a method for separation, using for instance antibodies coupled to fluorochromes for purification methods such as fluorescence activated cell sorting (FACS), and/or using for instance antibodies coupled to magnetic particles for magnetic selection procedures. Affinity purification methods are known in the art and can for instance be any method of separating biochemical mixtures based on a highly specific interaction such as that between antigen and antibody, enzyme and substrate, or receptor and ligand.

With the term “expanding” as used herein is meant multiplication of cells due to cell division events caused by a cell culturing step, preferably without essentially changing the phenotype of the cell, which is generally called “differentiation”. With the phrase “without essentially changing the phenotype of the cell” is meant that the cell preferably does not change its function, its cell surface markers and/or its morphology.

With the term “differentiating” as used herein is meant changing the phenotype of the cell, which means changing the expression of certain surface molecules during the cell culture process, changing the cells function and/or changing the morphology of the cell, wherein the cell preferably still can expand due to the addition of cell culture medium. As indicated previously, the inventors have shown that a composition for use immunotherapy as defined by the invention is particularly useful for the treatment of a tumor. According to a preferred embodiment, the composition for use according to the invention is for the treatment of a tumor. Tumor, within the meaning of the invention, includes hematopoietic tumors or solid tumors. The tumor can either be malign or benign.

A composition for use in immunotherapy according to the invention can be used at different stages in the treatment of tumors, in particular in the treatment of hematopoietic tumors, such as e.g. acute myelogenous leukemia (AML). For instance, as exemplified by the current invention, the composition can preferably be used as consolidation therapy in those (elderly) patients not eligible to undergo a bone marrow transplant. Additionally, as shown by others using another treatment, immune effector cell therapy according to the invention can preferably be used for patients not reaching complete remission on induction therapy (refractory patients), or those relapsing shortly after induction therapy (recurrent patients). Incorporation of immune effector cell therapy into other consolidation therapies is also feasible and preferred, such as the additional use of immune effector cells as defined by the invention in allogeneic HSTC regimens.

However, because of the shortcomings and problems with conventional haploidentical NK cell therapies and autologous T cell therapies, the present invention has developed a novel use of immune effector cells in immunotherapy, wherein the immune effector cells are preferably derived from batches of large numbers of highly activated immune effector cells through the ex vivo generation from CD34+ hematopoietic progenitor cells isolated from, e.g., UCB. Preliminary results of a phase I dose escalation study show safety, tolerability and the biological and clinical activity of the composition for use in the treatment of elderly (>55 yrs.) AML patients, which is a preferred group to be treated with a composition as defined by the invention.

In a working example, the composition was tested in elderly AML patients, who were given preparative chemotherapy consisting of cyclophosphamide (Cy;900 mg/m²/day) and fludarabine (Flu;30 mg/m²/day) on days −6 to −3. At day 0, UCB-derived immune effector cells at a dose of 3, 10 or up to 30×10⁶/kg body weight were infused without IL-2 treatment to study if in vivo expansion could be obtained without IL-2 support. Patients were assessed for toxicity and GVHD. As expected, preparative Cy/Flu induced a neutropenic period of 20±16 days, but no severe infections were seen.

In one embodiment, the invention provides cyclophosphamide for use in immunosuppressive therapy, characterized in that the cyclophosphamide is dosed on 2, 3, 4 or 5 subsequent days at a total dose of 400-10000 mg/m², preferably 800-8000, more preferably 1600-6000, more preferably 2000-4000, most preferably about 3600 mg/m², preferably concomitant with fludarabine at a total dose of 1-1000, preferably 10-500, more preferably 50-250, most preferably about 120 mg/m².

As used in the invention, cyclophosphamide is used in a reduced intensity compared to standard myeloablative regimens. Normally cyclophosphamide is also given in a higher concentration and less days than with this regimen. Remarkably, AML blasts are resistant to a certain level of cyclophosphamide treatment as they have aldehyde dehydrogenase (ALDH), which keeps cyclophosphamide away from being metabolized into its active form. As ALDH is not present in lymphocytes, cyclophosphamide will get active and deplete the cells.

Preferably the cyclophosphamide and/or the fludarabine are administered intravenously.

Fludarabine is acting as a purine analogue on resting and dividing cells, however it has a stronger effect on dividing cells at lower concentrations. Before the present invention, dosing was initially higher as it was used for targeting leukemic stem cells, which are resting cells and need a higher level of the drug to respond. Within the present invention, a lower dosage is used as a non-myeloablative regimen, causing much lower side effects.

The invention further provides fludarabine for use in immunosuppressive therapy, characterized in that the fludarabine is dosed on 2, 3, 4 or 5 subsequent days at a total dose of 1-1000, preferably 10-500, more preferably 50-250, most preferably about 120 mg/m², preferably concomitant with cyclophosphamide at a total dose of 400-10000 mg/m², preferably 800-8000, more preferably 1600-6000, more preferably 2000-4000, most preferably about 3600 mg/m².

In a preferred embodiment, cyclophosphamide for use according to the invention or fludarabine for use according to the invention is provided, wherein the fludarabine and cyclophosphamide are given prior to administration of a composition as defined by the invention. In particular the combination of fludarabine with cyclophosphamide as used herein leads to a better accumulation of cyclophosphamide in the stem cells (blasts), causing a potentially stronger effect. The conditioning with cyclophosphamide and fludarabine as described, prior to administration of a composition for use according to the invention has the effect that Immune effector cells of the patient are depleted in a milder way than using standard myeloablative conditioning regimens and that the rejection of the infused immune effector cells is prevented for a certain time period, given a potential effect on the tumor stem cells or make the more vulnerable for the infuse immune effector cells.

In a preferred embodiment, a composition for a use according to the invention is provided, wherein the composition to be administered in one treatment comprises at least 5×10⁶ cells, preferably at least 5×10⁷ cells, more preferably at least 5×10⁸, more preferably at least 5×10⁹ and most preferably at least 5×10¹⁰ and in any case, preferably not more than 5×10¹¹ cells. In a working example, the inventors have shown that doses in these ranges are safe and efficacious.

After infusion, UCB-derived immune effector cells repopulate, mature and migrate to BM without supporting IL-2 or IL-15 infusion. Since the inventors observed reduction in MRD in patients on treatment with hypomethylating agents, this UCB-derived immune effector cell therapy may induce or sustain CR in elderly AML patients, and could serve as an alternative consolidation therapy for patients with refractory AML or provide bridge to allo-SCT.

According to a preferred embodiment, a composition for a use according to the invention is provided, wherein the individual is not treated with IL-2 and/or IL-15.

In a preferred embodiment, a composition for a use according to the invention is provided, wherein the composition to be administered in one treatment comprises less than 2×10⁸ CD3 positive cells, more preferably less than 2×10⁷ CD3 positive cells, more preferably less than 2×10⁶ CD3 positive cells and most preferably less than 1×10⁵ CD3 positive cells.

In a preferred embodiment, a composition for a use according to the invention is provided, wherein the composition to be administered in one treatment comprises less than less than 1×10⁹ CD19 positive cells, more preferably less than 1×10⁸ CD19 positive cells, more preferably less than 1×10⁷ CD19 positive cells and most preferably less than 1×10⁶ CD19 positive cell.

In a preferred embodiment, the % of CD3 positive cells in relation to the number of total cells present in the composition does not exceed 10%, preferably 5%, more preferably 1%, more preferably 0.1%, and most preferably it does not exceed 0.01% in relation to the total number of cells present in the composition.

In a preferred embodiment, the % of CD19 positive cells in relation to the number of total cells present in the composition does not exceed 10%, preferably 5%, more preferably 1%, more preferably 0.1%, and most preferably it does not exceed 0.01% in relation to the total number of cells present in the composition.

The composition of the invention can be administered through any acceptable method, provided the immune effector cells are able to reach their target in the individual. It is for instance possible to administer the composition of the invention via the intravenous route or via a topical route, including but not limited to the ocular, dermal, pulmonary, buccal and intranasal route. With topical route, as used herein, is also meant any direct local administration such as for instance in the bone marrow, but also directly injected in, e.g., a solid tumor. In particular cases, e.g. if the immunotherapy is aimed at an effect on the mucosal layer of the gastrointestinal tract, the oral route can be used.

Preferably, a composition for a use according to the invention is provided, wherein the composition is administered by intravenous route or by a topical route or by oral route or by any combination of the three routes. With the term “topical” as used herein is meant, that the immune effector cells are applied locally, preferably at the site of tumor, which can be localized in any anatomical site, more specifically the tumor can be localized in the bone marrow or any other organ. The composition for use according to the invention can be administered once, but if deemed necessary, the composition may be administered multiple times. These can be multiple times a day, a week or even a month. It is also possible to first await the clinical result of a first administration, e.g. an infusion and, if deemed necessary, give a second administration if the composition is not effective, and even a third, a fourth, and so on.

As already elaborated before, a composition for use according to the invention is especially useful in immunotherapy for the treatment of a tumor. Without being bound to therapy, the HLA mismatched immune effector cell is thought to kill tumor cells through secretory lysosome exocytosis after recognizing its target. Target cell recognition induces the formation of a lytic immunological synapse between the immune effector cell and its target. The polarized exocytosis of secretory lysosomes is then activated and these organelles release their cytotoxic contents at the lytic synapse, specifically killing the target cell. The composition for use according to the invention for use in the treatment of a tumor is useful for both hematopoietic or lymphoid tumors and solid tumors. In a preferred embodiment, a composition according to the invention is provided, wherein the immune effector cell is able to kill a tumor cell through secretory lysosome exocytosis.

In one preferred embodiment, a composition for a use according to the invention for the treatment of a tumor is provided, wherein the tumor is a hematopoietic or lymphoid tumor or wherein tumor is a solid tumor.

With the term “hematological”, “hematopoietic” or “lymphoid” tumor is meant, that these are tumors of the hematopoietic and lymphoid tissues. Hematopoietic and lymphoid malignancies are tumors that affect the blood, bone marrow, lymph, and lymphatic system.

The present invention shows exemplary results for the effectiveness of a composition of the invention for use in both, the treatment of a hematopoietic and of solid tumors.

In those cases that the tumor is a hematopoietic or lymphoid tumor, a composition for use according to the invention is provided, wherein the tumor is one or more of leukemia, lymphoma, myelodysplastic syndrome or myeloma, preferably a leukemia, lymphoma or myeloma selected from acute myelogenous leukemia (AML), chronic myelogenous leukemia (CML), acute T cell leukemia, acute lymphoblastic leukemia (ALL), chronic lymphocytic leukemia (CLL), acute monocytic leukemia (AMoL), mantle cells lymphoma (MCL), histiocytic lymphoma or multiple myeloma, preferably AML.

In those cases that the tumor is a solid tumor, a composition for use according to the invention is provided, wherein the tumor is one of malignant neoplasms or metastatic induced secondary tumors of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumor, Waldenström macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma or retinoblastoma.

In a preferred embodiment, a composition for use according to the invention is provided, wherein the solid tumor is selected from malignant neoplasms or metastatic induced secondary tumors of cervical cancers selected from adenocarcinoma, squamous cell carcinoma, adenosquamous carcinoma, cervix carcinoma, small cell carcinoma, and melanoma. In another preferred embodiment, a composition for use according to the invention is provided, wherein the solid tumor is selected from malignant neoplasms or metastatic induced secondary tumors of colorectal cancers selected from adenocarcinoma, squamous cell carcinoma, colon adenocarcinoma, colorectal carcinoma, colorectal adenocarcinoma, colon carcinoma, and melanoma.

The composition of the invention has several advantages with respect to treatment options known to date. The composition of the invention is beneficial independent of HPV types, tumor histology, tumor EGFR expression and KRAS status. In addition to it, the immune effector cell of the invention also overcomes HLA-E, HLA-G and (IDO) inhibition, thus resulting in enhanced anti-tumor effects against tumors, especially against cervical cancers and colorectal cancers.

The term “Epidermal growth factor receptor” or EGFR as it is commonly described, refers to a cell surface protein widely expressed in almost all healthy tissues. The EGFR protein is encoded by transmembrane glycoprotein and is a member of the protein kinase family. Overexpression of EGFR and mutations in its downstream signaling pathway has been associated with bad prognosis in several solid tumors like colon, lung and cervix.

The term Kirsten rat sarcoma viral oncogene (KRAS) refers to the gene actively involved in regulating normal tissue signaling, part of EGFR downstream signaling pathway. However, mutations in the KRAS gene has been reported in tumor cells in solid tumors of colon, rectum and lungs. This activating mutations occurring in more than 50% of colorectal cancer patient helps tumor cells to evade EGFR targeting drugs like cetuximab and panitumumab.

The term “human papilloma virus (HPV) as used herein refers to the group of viruses which causes cervical cancer in women. HPV virus affects the skin and moist membranes surrounding mouth, throat, vulva, cervix and vagina. HPV infection causes abnormal cell changes that leads to cancer in the cervix.

The term Indoleamine 2,3 dioxygenase (IDO) as used herein refers to an enzyme which acts as a catalyst in degrading amino acids L-tryptophan to N-formylkynurenine. Overexpression of IDO commonly reported in solid tumors of prostate, gastric, ovarian, cervix and colon, enables tumor cells to evade killing by cytotoxic T cells and NK cells.

For those jurisdictions that allow claims on medical treatment, the following embodiments are also provided by the invention:

Method for treating an individual in need of immunotherapy, the method comprising administering to the individual a composition comprising an immune effector cell, characterized in that the immune effector cell is non-haploidentical with respect to the individual.

Method for treating an individual in need of immunotherapy according to the invention, wherein the immune effector cell is positive for Neural Cell Adhesion Molecule (NCAM) and negative for CD3 and CD19.

Method for treating an individual in need of immunotherapy according to the invention, wherein the immune effector cell expresses one or more of the following cell surface markers: CD159a, CD314, CD335, CD336, CD337.

Method for treating an individual in need of immunotherapy according to the invention, wherein the immune effector cell expresses CD314, CD336, or both.

Method for treating an individual in need of immunotherapy according to the invention, wherein the composition comprises a plurality of cells, characterized in that 30-100%, preferably 30-90%, more preferably 30-80%, more preferably 30-70%, more preferably 30-60%, more preferably 30-50%, most preferably 30-40% of the plurality of cells is an immune effector cell as defined in the invention.

Method for treating an individual in need of immunotherapy according to the invention, wherein the composition comprises a plurality of cells, characterized in that 40-100%, more preferably 50-100%, more preferably 60-100%, more preferably 70-100%, more preferably 80-100%, most preferably 90-100% of the plurality of cells is an immune effector cell as defined in the invention.

Method for treating an individual in need of immunotherapy according to the invention, wherein the immunotherapy is for the treatment of a tumor.

Method for treating an individual in need of immunotherapy according to the invention, wherein the immune effector cell is generated ex vivo from a stem cell.

Method for treating an individual in need of immunotherapy according to the invention, wherein the immune effector cell is generated ex vivo from a progenitor cell.

Method for treating an individual in need of immunotherapy according to the invention, wherein the stem cell is a CD34+ stem cell.

Method for treating an individual in need of immunotherapy according to the invention, wherein the progenitor cell is a CD34+ progenitor cell.

Method for treating an individual in need of immunotherapy according to the invention, wherein the individual is not treated with IL-2 and/or IL-15.

Method for treating an individual in need of immunotherapy according to the invention, wherein the plurality of cells are derived from cells obtained from a single donor.

Method for treating an individual in need of immunotherapy according to the invention, wherein the plurality of cells are derived from at least one of umbilical cord blood and bone marrow.

Method for treating an individual in need of immunotherapy according to the invention, wherein the composition is generated ex vivo in a process comprising the steps of:

obtaining a sample comprising CD34+ hematopoietic stem and/or progenitor cells

affinity purification of CD34+ hematopoietic stem and progenitor cells from the sample obtained in a);

expanding the purified CD34+ hematopoietic stem and progenitor cells obtained in b) in a basal growth medium supplemented with human serum, a low-dose cytokine cocktail consisting of three or more GM-CSF, G-CSF, LIF, MIP-1α and IL-6, a specific combination of two or more of high-dose cytokines including SCF, Flt3L, IL-7 and TPO and a low-molecular weight heparin; and,

differentiating the expanded CD34+ hematopoietic stem and progenitor cells obtained in c) in a basal growth medium supplemented with human serum and IL-15 and additional one or more cytokines including SCF, Flt3L, IL-7, IL-12, IL-18 and IL-2,

harvesting the cells generated in d) and generating a composition as defined in any one of claims 1-14.

Method for treating an individual in need of immunosuppressive therapy, the method comprising administering cyclophosphamide and/or fludarabine to said individual, characterized in that the cyclophosphamide is dosed on 2, 3, 4 or 5 subsequent days at a total dose of 400-10000 mg/m², preferably 800-8000, more preferably 1600-6000, more preferably 2000-4000, most preferably about 3600 mg/m², and/or the fludarabine is dosed on 2, 3, 4, or 5 subsequent days at a total dose of 1-1000, preferably 10-500, more preferably 50-250, most preferably about 120 mg/m².

Method for treating an individual in need of immunosuppressive therapy according to the invention, wherein the fludarabine and cyclophosphamide are given prior to administration of a composition as defined in the invention.

Method for treating an individual in need of immunotherapy according to the invention, wherein the composition to be administered in one treatment comprises at least 5×10⁸ cells.

Method for treating an individual in need of immunotherapy according to the invention, wherein the composition to be administered in one treatment comprises not more than 1×10¹⁰ cells.

Method for treating an individual in need of immunotherapy according to the invention, wherein the composition to be administered in one treatment comprises less than 2×10⁸ CD3 positive cells.

Method for treating an individual in need of immunotherapy according to the invention, wherein composition to be administered in one treatment comprises less than 1×10⁹ CD19 positive cells.

Method for treating an individual in need of immunotherapy according to the invention, wherein the composition is administered by intravenous route.

Method for treating an individual in need of immunotherapy according to the invention, wherein the composition is administered by a topical route.

Method for treating an individual in need of immunotherapy according to the invention, wherein the tumor is a hematopoietic or lymphoid tumor or wherein tumor is a solid tumor.

Method for treating an individual in need of immunotherapy according to the invention, wherein the tumor is a hematopoietic or lymphoid tumor, selected from leukemia, lymphoma, myelodysplastic syndrome or myeloma, preferably a leukemia, lymphoma or myeloma selected from acute myelogenous leukemia (AML), chronic myelogenous leukemia (CML), acute T cell leukemia, acute lymphoblastic leukemia (ALL), chronic lymphocytic leukemia (CLL), acute monocytic leukemia (AMoL), mantle cells lymphoma (MCL), histiocytic lymphoma, multiple myeloma, any others?.

Method for treating an individual in need of immunotherapy according to the invention, wherein the leukemia is AML.

Method for treating an individual in need of immunotherapy according to the invention, wherein the tumor is a solid tumor, selected from malignant neoplasms or mestastatic induced secondary tumors of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumor, Waldenström macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma and retinoblastoma.

In a preferred embodiment, a method according to the invention is provided, wherein the solid tumor is selected from malignant neoplasms or metastatic induced secondary tumors of cervical cancers selected from adenocarcinoma, squamous cell carcinoma, adenosquamous carcinoma, cervix carcinoma, small cell carcinoma, and melanoma.

In another preferred embodiment, a method according to the invention is provided, wherein the solid tumor is selected from malignant neoplasms or metastatic induced secondary tumors of colorectal cancers selected from adenocarcinoma, squamous cell carcinoma, colon adenocarcinoma, colorectal carcinoma, colorectal adenocarcinoma, colon carcinoma, and melanoma.

The invention is described in more detail in the following, non-limiting examples.

### BRIEF DESCRIPTION OF DRAWINGS

FIG. 1: Clinical study set up

Acute myeloid leukemia (AML) patients above 55 years, who are not eligible for stem cell transplantation (SCT) received a standard remission induction chemotherapy (RIC) treatment. Patients that received clinical remission (CR) were eligible to participate in the immunotherapy study. The immunotherapy product of this invention was given in escalating doses after an immunosuppressive preparative treatment with cyclophosphamide (Cy) and fludarabine (Flu). The product comprises of HLA mismatched immune effector cells, which were applied in escalating doses in order to evaluate safety and toxicity of this product. Further, biologic function such as in vivo survival, expansion and effect on MRD was studied.

FIG. 2: Toxicity

Patients are treated at day −6, −5, −4 and −3 with cyclophosphamide and fludarabine before getting the cell treatment at day 0. The graph shows the reduction in Neutrophil counts for individual patients in A and (schematically) for the whole study population in B. No observation of toxicities after cell infusion have been reported. Mainly the expected hematological toxicities due to the immunosuppressive regimen has been reported. Most patients had fast neutrophil recovery after 14 days as shown for individual patients (A) and the whole study population (B).

FIG. 3: Lymphodepletion and IL-15 levels

The number of Leukocytes has been followed after Cy/Flu conditioning. The conditioning resulted in a decrease of Leukocytes of all individual patients which goes side by side with the increase in IL-15 levels (line with squares; see legend as indicated with the arrow). After 14 days both lines reached about the steady state conditions again.

FIG. 4: Donor cell chimerism in whole blood and bone marrow

Donor cell chimerism is analyzed by SNP-PCR based on % donor DNA present in whole blood sample or bone marrow sample. Chimerism of infused cell products was followed over time. In peripheral blood (A) chimerism of individual patients could be detected up to 14 days. Corresponding chimerism has been found in bone marrow (B) as well.

FIG. 5: Circulation of infused immune effector cells

Infused immune effector cells are detected in patients peripheral blood by the high expression of NCAM (quadrant as indicated by the arrow) and separated from the patient's own effector cells like NK cells or T cells. Cells were analyzed by flow cytometry. A representative example is shown here. Before infusion no effector cells can be detected. 4 hours after infusion (day 0+4) the immune effector cells can be detected in the blood. During the time the cells persists and expand till day 8.

FIG. 6: Reduction of MRD (UPN7)

In UPN7 a potential clone of leukemic blasts was described by Leukemic associated phenotype (LAP) CD45+/CD34+/CD117−/CD133+. After immunotherapy using the cell product of this invention, a reduction in leukemic blast count from 6.7% towards an undetectable limit <0.01% could be observed.

FIG. 7: Reduction of MRD (UPN8)

In UPN8 a potential clone of leukemic blasts was described by Leukemic associated phenotype (LAP) CD45+/CD34+/CD7+/CD133+. After immunotherapy using the cell product of this invention, a reduction in leukemic blast count from 6.3% towards and a nearly undetectable limit of 0.02% could be observed.

FIG. 8: Overall survival

The survival of all patients treated was followed beyond the study limit of 180 days. Till date, 4 from 10 patients died. Compared to historic control group of AML patients (survival % indicated by *) with age 65-74, the relative survival seems to improve significantly). Control data taken from the Netherlands Cancer Registry (www.dutchcancerfigures.nl)

FIG. 9: Progression free survival

The progression free survival was followed beyond the study limit of 180 days. 50% of the 10 patients relapsed so far, from which 1 patient relapse later than 1 year after treatment. 4 patient relapsed between 5-7 months after treatment.

FIG. 10: Cytotoxicity of ex vivo generated effector cells vs. epidermoid carcinoma

Immune effector cells (UCB-EC) as described in this invention are capable of killing epidermoid carcinoma cells (A431), as indicated by the percentage of 7-Aminoactinomycin D (% 7AAD), more efficient than activated Natural Killer cells from peripheral blood (PBNK). *** indicates p<0.001.

FIG. 11: Cytotoxicity of ex vivo generated effector cells vs. colon cancer

Immune effector cells (UCB-EC) as described in this invention are capable of killing colon cancer cells more efficiently than activated Natural Killer cells from peripheral blood (PBNK) irrespectively of RAS or BRAF status, as indicated by the percentage of 7-Aminoactinomycin D (% 7AAD).

FIG. 12: Cytotoxicity of ex vivo generated effector cells vs. cervical cancer

Immune effector cells (UCB-EC) as described in this invention are capable of killing cervical cancer cells more efficiently than activated Natural Killer cells from peripheral blood irrespectively of HPV status and type, as indicated by the percentage of 7-Aminoactinomycin D (% 7AAD).

FIG. 13: Cytotoxicity of ex vivo generated effector cells vs. hematopoietic cancer

Immune effector cells as described in this invention are capable of killing hematological cancer cells such as leukemia (K562) or multiple myeloma (U266).

FIG. 14: Cytotoxicity of ex vivo generated effector cells vs. liquid and solid tumors

Immune effector cells as described in this invention are capable of killing hematological cancer cells, as indicated by the percentage of 7-Aminoactinomycin D (% 7AAD) and show high activity (measured by the degranulation of cytotoxic granules using CD107a (LAMP1) expression) against acute lymphoblastic leukemia (CCRF-CM, MOLT-4), pancreatic cancer (Mia-Pa-Ca-2), or lung cancer (NCI-H82) (small cell lung cancer).

FIG. 15: Expression of FcRγIIIa on immune cell product

FcRγIIIa expression was analyzed on the immune cell product use in the clinical study. The results show variable expression of FcRγIIIa. Measured values are summarized in the graph and displayed in the table. Average, max, min value and standard deviation (SD) has been calculated.

FIG. 16: Comparison of expression of FcRγIIIa on immune cell product and peripheral blood natural killer cells

FcRγIIIa (=CD16a) expression was analyzed on the immune cell product as used for the cytotox experiments. The results show low expression of FcRγIIIa compared to expression levels of stimulated and non-stimulated natural killer cells from peripheral blood. Measured values are summarized in the graph. Average, max, min value and standard deviation (SD) has been calculated. In the table the UPN (unique patient number) number represents the specific effector cell product, this specific patient received.

FIG. 17: Comparison of IL-12 and 2 in various combinations during differentiation phase

Culture procedure for culturing UCB-EC cells from UCB derived CD34+ cells UCB derived CD34+ cells are cultured for 2 weeks in expansion medium I. Progenitors are next cultured in differentiation I medium with a high-dose cytokine combination of IL-15, SCF and IL-7. Additional IL-2 and/or IL-12 cytokines are added to the culture medium at 3 different time points: after week 2, 3 or 4. 12 culture conditions were used as coded on the right. Underscore (_) mean the passage of a week from week 2 to 3 or 3 to 4 and the minus sign (-) means no additional cytokine is added that week.

FIG. 18: UCB-EC overcomes tumor HLA ABC inhibition significantly higher than PBNK cells

Representative histogram plots showing geometric mean fluorescence intensity (MFI) of NK inhibitory ligands HLA-ABC, HLA-E and HLA-G on cervical cancer cells; representative plots of 2-3 separate analyses are shown (A). Correlation analysis of MFI of HLA-ABC with % cytotoxicity (Δ7AAD) by (B) PBNK, (C) PBNK+cetuximab, and (D) UCB-EC. Dotted lines represent 95% confidence interval of the regression line. P-value was calculated with Pearson analysis.

FIG. 19: Activated UCB-EC cells overcome tumor HLA-E inhibition

Cytotoxicity of UCB-EC cells against HLA-E overexpressing cell lines Siha, CC10a and Caski were tested co-culturing UCB-EC with targets at a ratio of E:T 1:1. Target cell death (A) and UCB-EC degranulation (B) were quantified to determine UCB-EC ability to lyse tumor targets in comparison with activated PBNK. Similarly, in the next level, Target cell death (C) and UCB-EC degranulation (D) was compared to PBNK+CET conditions. Data presented is from four individual PBNK (shaded bars) and five UCB-EC (hatched bars) donors; bars represent SEM. Mean±SEM for are calculated using one way anova and each significant condition are represented as p=<0.05 *, <0.01 **, <0.005 ***, <0.001 ****.

FIG. 20: Activated UCB-EC cells overcome tumor HLA-G inhibition

From our panel of screened cervical cancer cell lines, Siha, CC10A, CC8 and CC10B expressed high levels of HLA-G. Ability of UCB-EC to initate tumor lysis against these targets were measured by quantifying the percentage of dead cells (A) and UCB-EC degranulation (B) and compared to activated PBNK. Further the same was compared to PBNK+CET conditions as shown in figure C and D. Data presented is from four individual PBNK (shaded bars) and five UCB-EC (hatched bars) donors; bars represent SEM. Mean±SEM are calculated using one way anova and each significant condition are represented as p=<0.05 *, <0.01 **, <0.005 ***, <0.001 ****.

FIG. 21: Indefinite killing of cervical tumors by UCB-EC is independent of HPV types

Cytotoxicity of UCB-EC and PBNK cells alone and PBNK+cetuximab (CET) were compared grouping ten cervical cancer cell lines based on different HPV types. PBNK (open bars), PBNK+(CET) (closed bars), and UCB-EC (hatched bars) cytotoxicity levels according to HPV type of cervical cancer cell lines. Bars represent mean±SEM. Higher killing of UCB-EC compared to PBNK and PBNK+CET conditions in HPV16 and HPV18 are denoted by *.

FIG. 22: Cervical tumor killing by UCB-EC and PBNK cells is independent of tumor histology

Ten cervical cancer cell lines used in the study were categorized according to their histological origins.

UCB-EC, PBNK alone and PBNK+cetuximab ability to initiate tumor cell lysis was measured. PBNK (open bars), PBNK+cetuximab (CET) (closed bars), and UCB-EC (hatched bars) cytotoxicity levels according to histological classification. Bars represent mean±SEM. AC: adenocarcinoma; SCC: squamous cell carcinoma; ASC: adenosquamous cell carcinoma. Bars represent mean±SEM, Higher killing of UCB-EC compared to PBNK and PBNK+CET conditions in squamous cell carcinoma and epidermoid carcinoma cell types are denoted by *.

FIG. 23: Cetuximab monotherapy against EGFR expressing and RAS^{wt} cervical cancer cell lines

Ten cervical cancer cell lines were incubated with 5 μg/m/cetuximab for 4 hrs at 37° C. and tested for sensitivity towards anti-EGFR monoclonal antibody cetuximab by monitoring cell death using 7AAD marker. The data presented is from three independent experiments. Bars represent mean±SEM. P values are calculated using two way anova with multiple comparison between column means. Mean±SEM are calculated using one way anova and each significant condition are represented as p=<0.05 *, <0.01 **, <0.005 ***, <0.001 ****.

FIG. 24: UCB-EC killing independent of tumor EGFR and RAS types.

UCB-EC and PBNK were co-cultured with cervical cancer cell lines expressing varying levels of EGFR. Cytotoxicity assays were performed incubating cervical cancer targets with UCB-EC and PBNK and measured for their ability to lyse EGFR high, low and negative cell lines. (A) Cytotoxicity levels (Δ7AAD) of PBNK (open bars) and UCB-EC (hatched bars) against ten cervical cancer cell lines. Bars are means of triplicate values from four experiments for C33A, HeLa, SiHa, CC11B, CC11A, CC10B, CC10A, CaSki and two experiments for CSCC7 and CC8 using PBNK and five experiments using UCB-EC for all cell lines; Bars represent mean±SEM, calculated using Student's T test. Statistically significant (p=<0.05) UCB-EC cytotoxicity compared to PBNK and PBNK+CET conditions are denoted by *.

FIG. 25: Comparison of UCB-EC and PBNK cytotoxicity against cervical cancer cells

Means of triplicate values from four experiments for C33A, HeLa, SiHa, CC11B, CC11A, CC10B, CC10A, CaSki and two experiments for CSCC7 and CC8 using PBNK and five experiments using UCB-EC for all cell lines as shown in figure (A). Significantly higher cytotoxicity levels (Δ7AAD) were observed in all cell lines after co-culture with UCB-EC compared to PBNK. **P<0.01 and ***P<0.005 calculated with paired student's t test.

FIG. 26: UCB-EC killing and functionality is comparable to PBNK+CET conditions

Means of triplicate values from four experiments for C33A, HeLa, SiHa, CC11B, CC11A, CC10B, CC10A, CaSki and two experiments for CSCC7 and CC8 using PBNK and PBNK+CET conditions and five experiments using UCB-EC for all cell lines as shown in figure (A). Significantly higher levels of NK degranulation (ΔCD107a) were seen in PBNK+CET and UCB-EC conditions compared to PBNK only condition. Triangles denote cell lines with low EGFR levels, i.e. C33A, HeLa, and SiHa. **P<0.01 calculated with one-way ANOVA, Bonferroni's multiple comparison test.

FIG. 27: UCB-EC killing mechanism dependent on DNAM-1 and NKG2D similar to PBNK cells

Representative example of histograms showing geometric mean fluorescence intensity (MFI) for NK activating ligands PVR (ligand of DNAM-1 receptor), MICA/B, and ULBP1, -3 and -2/5/6 (ligands of NKG2D receptor) shown in figure A. (B) PBNK and UCB-EC were coated with NKG2D and/or DNAM-1 blocking antibodies and incubated with C33A and SiHa cells. Cytotoxicity levels (Δ7AAD) were measured from 7AAD+ C33A and SiHa cells at the end of a 4h assay. Data presented are means of triplicate values from three independent experiments; Bars represent mean±SEM. * P<0.05 and ** P<0.01 calculated with paired, two-way ANOVA multiple comparisons of column means.

FIG. 28: UCB-EC cells overcome IDO inhibitory effects of cervical cancer cells

Immune effector cells (UCB-EC) as described in this invention are capable of killing cervical cancer cells Caski and Siha, which overexpress the inhibitory IDO and also at a higher level than activated PBNK cells as indicated by their percentage of 7AAD positive target cells. Data presented are means of triplicate values from four independent experiments; Bars represent mean±SEM. * P<0.05 and *** P<0.005 calculated with one-way ANOVA multiple comparisons of column means.

FIG. 29: Comparison of in vitro cytotoxic efficacy of A-PBNK and UCB-NK cells against CRC cells.

(A) CRC cell lines of varying EGFR expression levels and different RAS and BRAF status, COLO320 (EGFR−, RASwt), SW480 (EGFR+, RASmut) and HT-29 (EGFR+, RASwt, BRAFmut) were subjected to NK killing using two allogeneic NK cell products, A-PBNK and UCB-NK cells. NK cell cytotoxicity assays were performed, incubating tumor cells with NK cells at an E:T ratio 1:1 for 4h at 37° C. CRC cell lines were used either coated with or without cetuximab to measure NK ADCC effects. 7AAD was used to determine target cell death (A) and CD107a to quantify NK degranulation upon target stimulation (B). Data presented here is from 5 PBNK and UCB-NK healthy donors. Experiments were done in triplicates. Bars represent mean±SEM. *P<0.05 and **P<0.01, calculated with two-way ANOVA, multiple comparison between column means.

FIG. 30: Experimental time line and study design for UCB-NK cells and cetuximab combinatorial studies in vivo.

Twenty-four BRGSwt mice were divided among control and treatment groups. SW480 (A) is the control group, followed by treatment groups SW480+cetuximab (B), SW480+UCB-NK (C) and SW480+UCB−NK+cetuximab (D). 0.5×10⁶ per mice Gluc transduced SW480 cells were administered intravenously to all groups at day 0. On day 1 (I dose) post tumor injection, Groups B and D mice were administered with 0.5 mg cetuximab per mice intraperitoneally and Groups C and D were infused intravenously with 10×10⁶ UCB-NK cells. Same concentration of cetuximab and UCB-NK cells were repeated on day 3 (II dose) and day 7 (III dose) for the respective groups, thus totaling cetuximab 1.5 mg per mice and NK cell infusions upto 30×10⁶ per mice. 0.5 μg IL-15 was mixed with 7.5 μg IL-15 receptor alpha (IL-15Ra) and were administered to the UCB-NK cell groups on days 1, 4, 7, 10 and 14. Treatment effects were monitored using blood Gluc levels, by drawing 25 μl of blood twice a week and further tumors were imaged on Day 35.

FIG. 31: Significant anti-tumor effects of UCB-NK cells indicated by blood Gluc assays.

Real time monitoring of tumor progression and treatment response was done measuring Gluc levels from mice blood twice a week. Baseline Gluc values were obtained from all mice a day (day −1) before tumor injection, and further monitoring continued till day 35. 25 μl of blood was collected from the tail vein on days −1, 4, 7, 10, 14, 17, 20, 24, 30 and 35 post tumor injection and Gluc activity was acquired using a calibrated luminometer. For statistical analysis, groups with similar blood Gluc levels, SW480 only and SW480+cetuximab were grouped as one and compared with the combined data from SW480+UCB-NK and SW480+UCB-NK+cetuximab groups. Gluc levels were significantly decreased in UCB-NK treatment groups, and with no difference observed between UCB-NK and UCB-NK+ cetuximab groups. Data presented is from 6 mice per group (n=6). Scatter plots represent mean±SEM. **P<0.013, calculated with one-way ANOVA.

FIG. 32: Successful tumor elimination by UCB-NK cells revealed by bioluminescence imaging in vivo

Four mice from control and treatment groups were imaged at day 35 for tumor growth. Mice were injected retro-orbitally with Gluc substrate coelenterazine and images were acquired for 5 min. (A) In SW480 control and SW480+cetuximab groups, tumor growth was extensive and were highly disseminated spreading to most parts of the body, however in UCB-NK and UCB-NK+ cetuximab groups there was a significant reduction in the tumor load, which was further verified by calculating the average radiance between groups as shown in figure B (n=4 mice per group). (C) Cetuximab functionality against EGFR+++ RASwt A431 cells was tested in parallel to SW480 studies in BRGSwt mice (n=3 mice per group). For figures B and C, bars represent mean±SEM. ***P<0.005 for figure B was calculated with one-way ANOVA, multiple comparison between column means and for figure C using paired t-test.

FIG. 33: Significant survival benefit in cetuximab resistant RAS mutant tumor bearing mice treated with UCB-NK cells

Kaplan-Meier survival curves were plotted for the total experimental study period from day 0 till day 65. SW480 (EGFR+, RASmut) tumor bearing mice (n=6 per group) following treatment with PBS only (black), cetuximab only (blue), UCB-NK only (green) and UCB-NK+cetuximab (orange) on days 1, 4 and 7 post tumor injection. Survival advantage for UCB-NK treatment groups was statistically significant compared to PBS control and cetuximab treated groups. Statistical differences between groups were calculated using log rank (Mantel-Cox) test and indicated at the bottom of the figure.

### DETAILED DESCRIPTION

### Examples

### Example 1

Ex vivo-generated allogeneic immune effector cells are infused into poor-prognosis acute myeloid leukemia (AML) patients following cyclophosphamide/fludarabine (Cy/Flu) conditioning. This immunosuppressive conditioning regimen is necessary to prevent rejection and has shown to induce immune effector cell survival factors such as IL-15 that facilitate prolonged in vivo lifespan and expansion of the infused immune effector cells. The immune effector cell products are >70% for Neural Cell Adhesion Molecule (NCAM) expression and almost devoid of CD3+ T cells, thereby minimizing donor T cell-mediated GVHD. Study participants will undergo clinical and immunological evaluation. After achieving complete remission (<5% blasts in bone marrow) following one or two induction chemotherapy courses patients are typed for HLA class I alleles by serological testing and polymerase chain reaction (PCR-SSOP) and tested for the absence of anti-HLA antibodies using a standard Luminex protocol. Eligible AML patients are those without anti-HLA antibodies and for whom a allogeneic non-haploidentical UCB unit displaying an available HLA match for HLA-A and HLA-B at antigen level can be found in a pool of 50 randomly selected UCB units. HLA-DRB1, HLA-DQ and HLA-DP matching have not been used for UCB unit selection. Immediately after allocation, while consolidation chemotherapy is performed according to standard protocol, available UCB units are screened for selecting an appropriate donor for ex vivo immune effector cell expansion.

Six weeks prior to immune effector infusion, the suitable allogeneic UCB unit is thawed and CD34+ cells are enriched by using a CliniMACS cell separator after binding with CD34 coupled to immunomagnetic particles (Miltenyi Biotec). Enriched CD34+ UCB cells are used for ex vivo generation of NCAM positive immune effector cell products, through differentiation and expansion, according to the validated procedure⁷². Cell isolation, enrichment and culture procedures are performed under Good Manufacturing Practice (GMP) conditions in a clean room, using established SOPs according to JACIE, NETCORD FACT guidelines and EU directive 2001/83 and 2009/120.

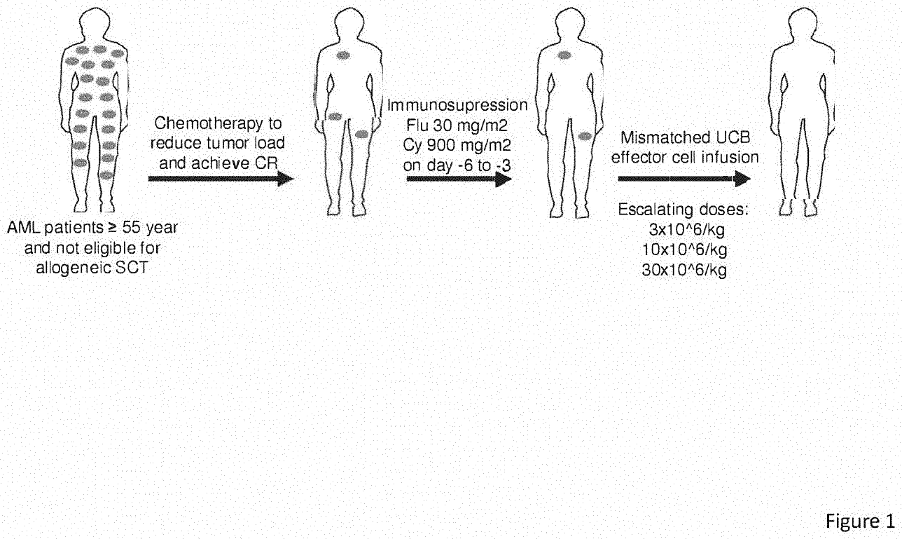

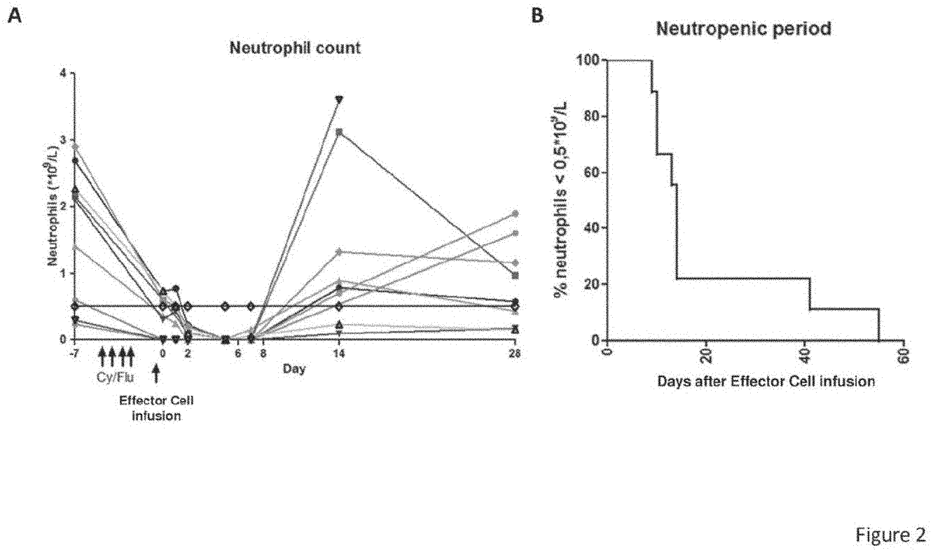

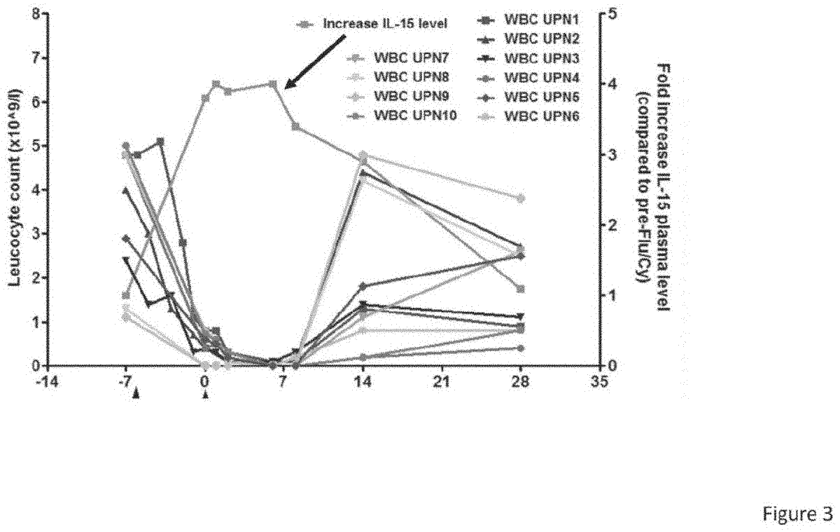

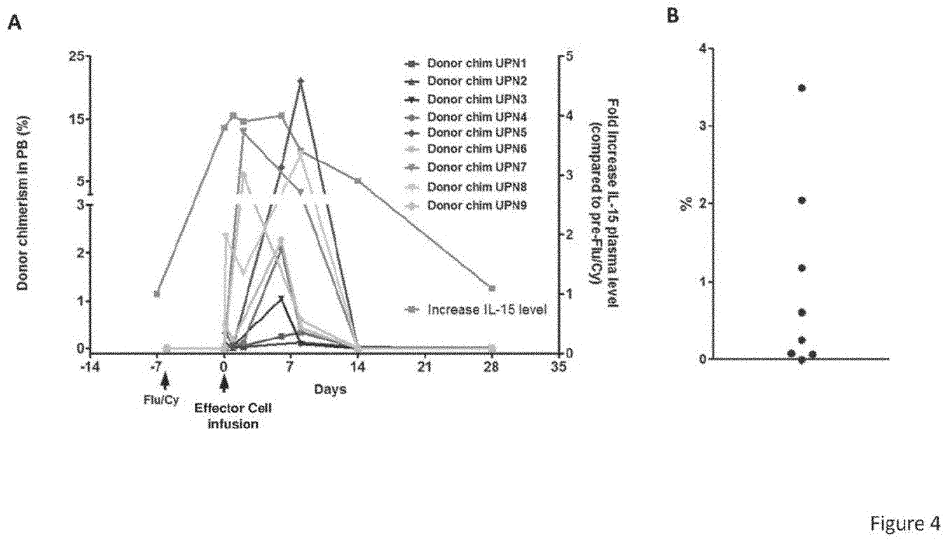

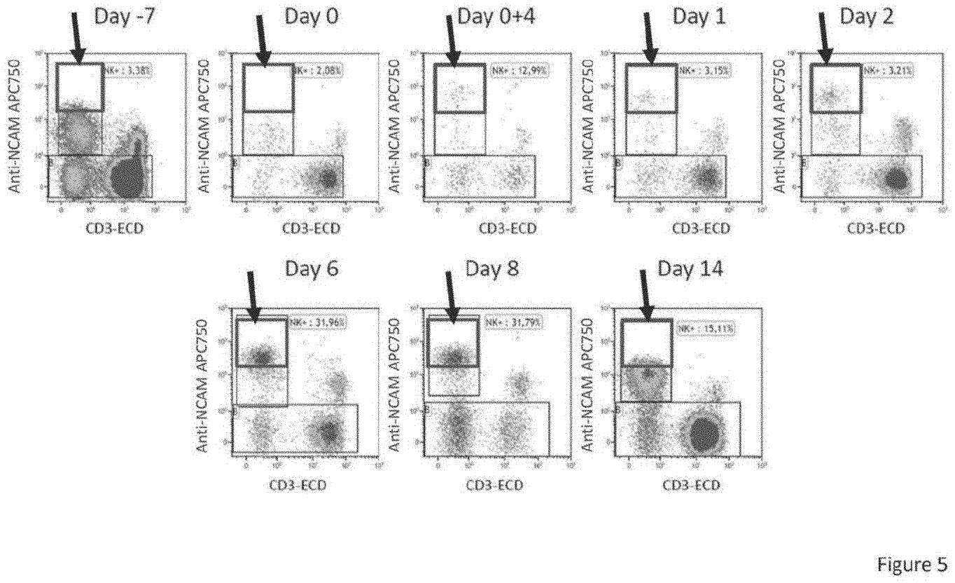

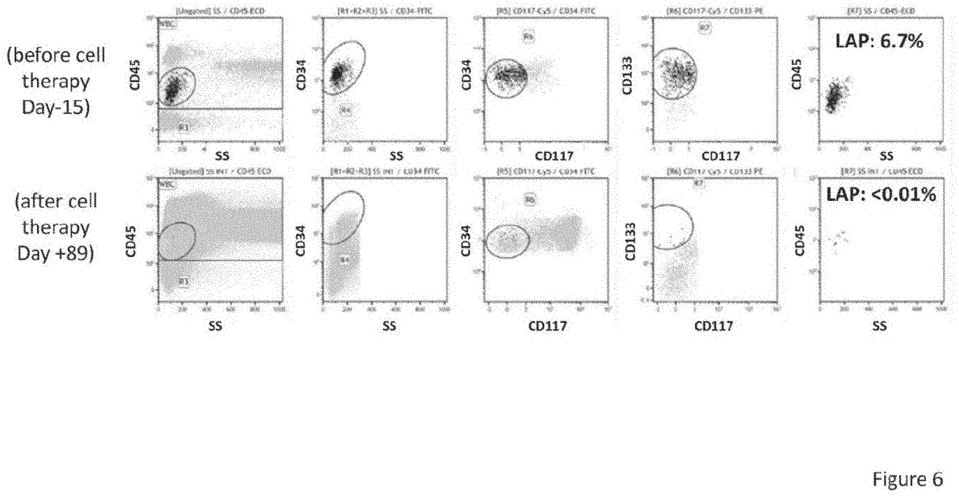

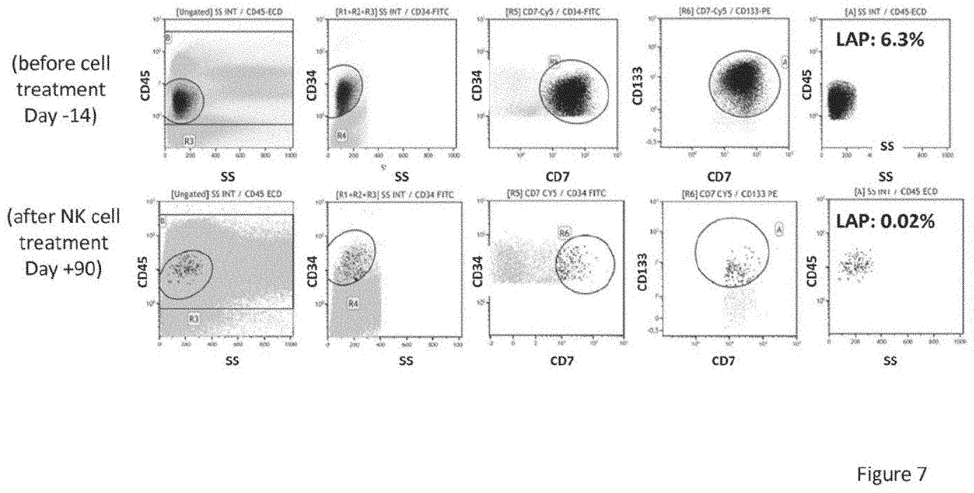

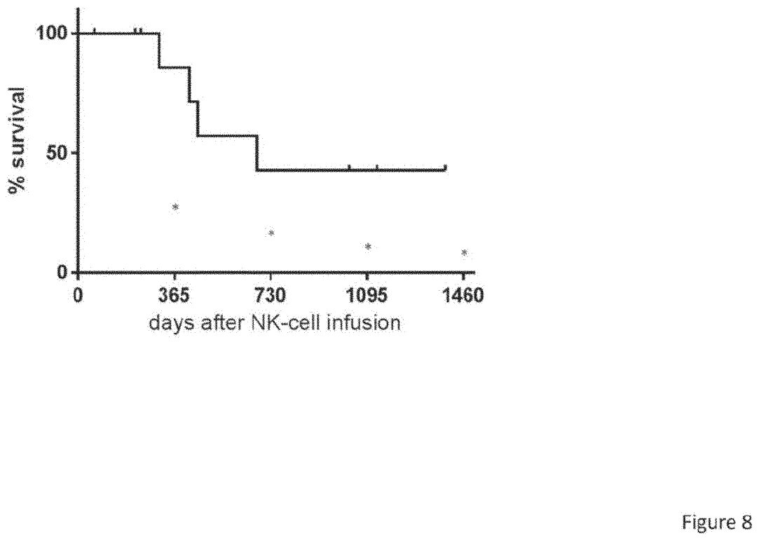

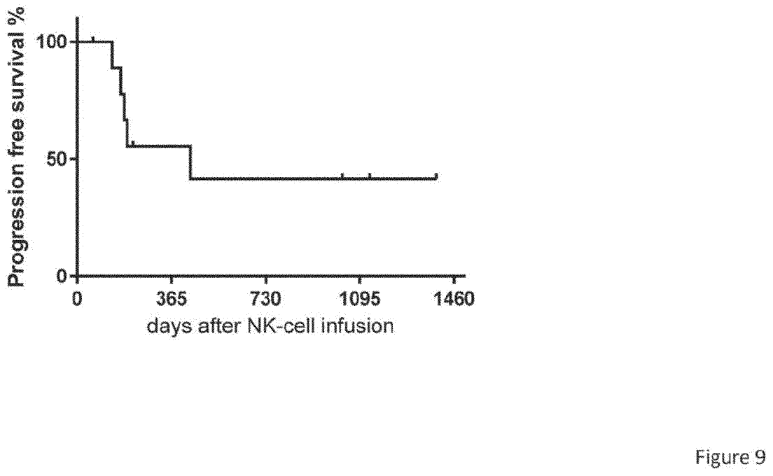

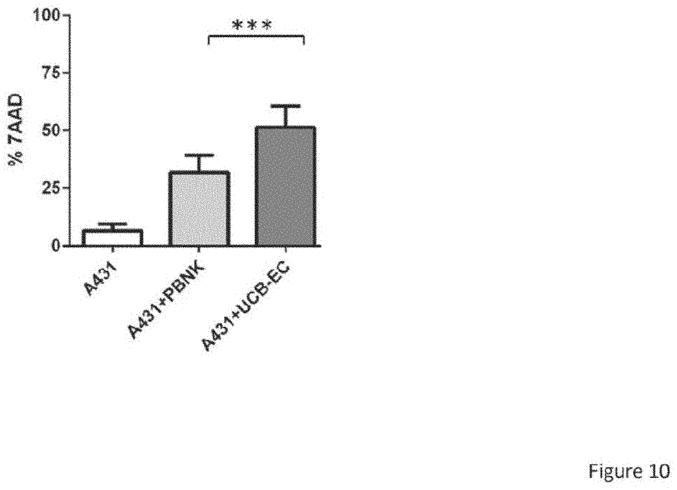

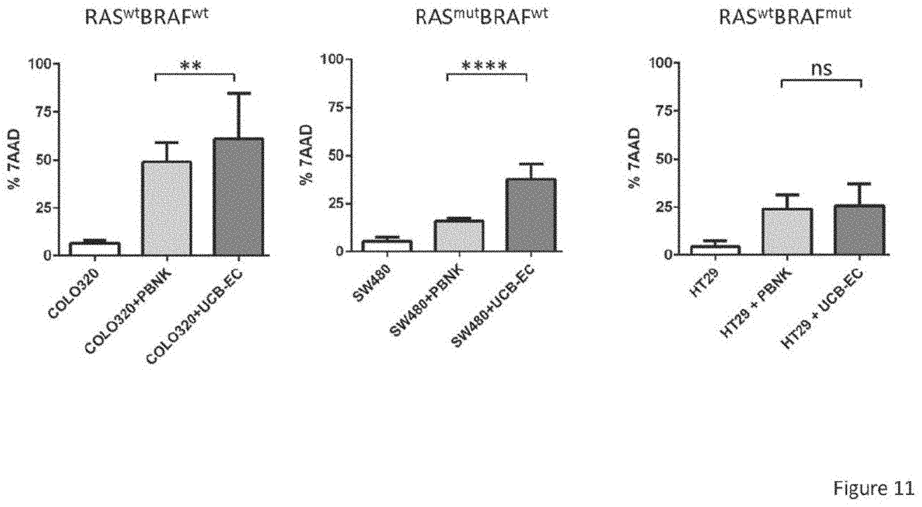

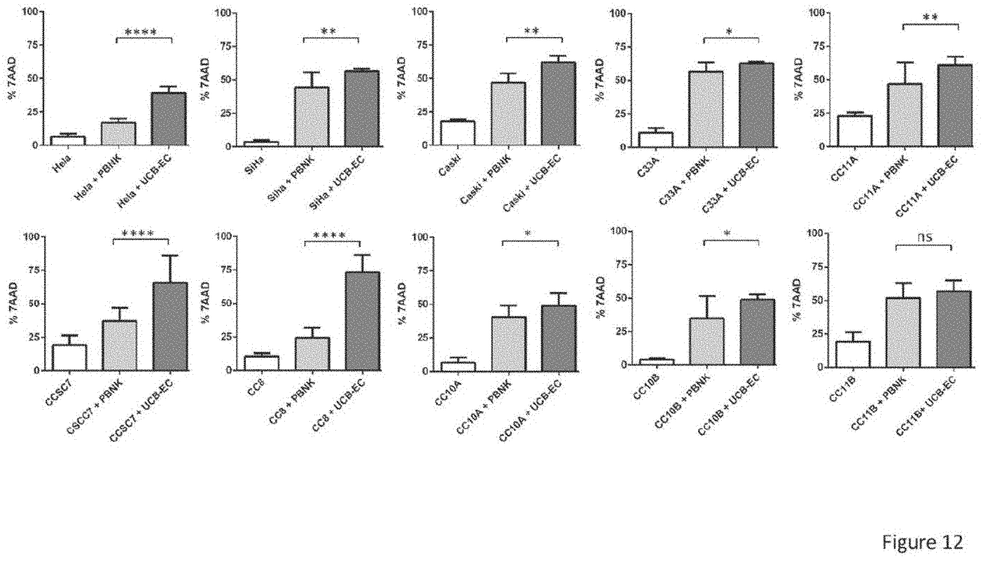

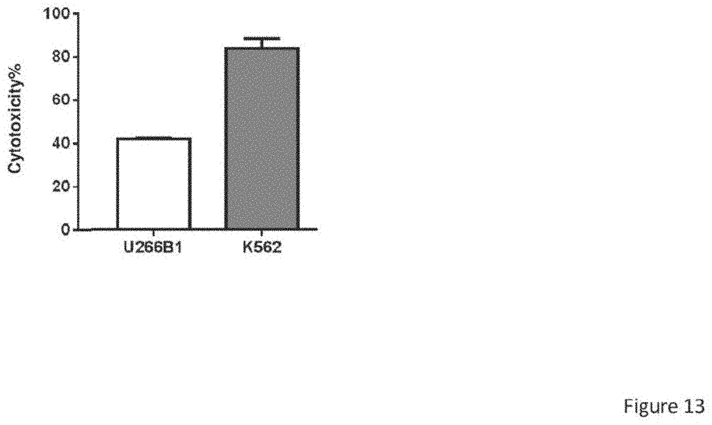

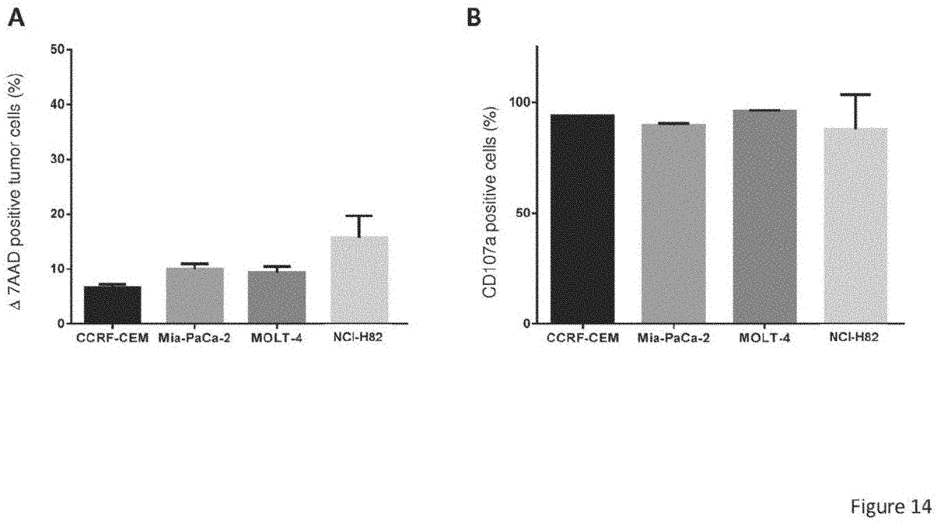

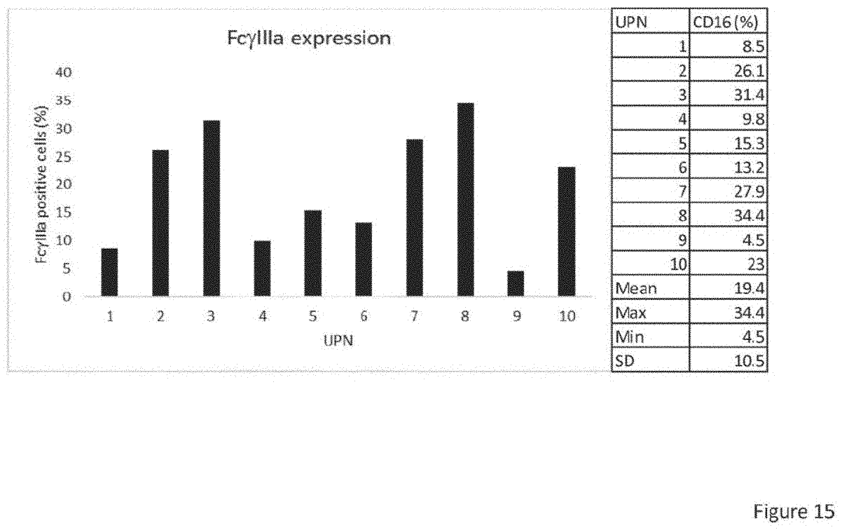

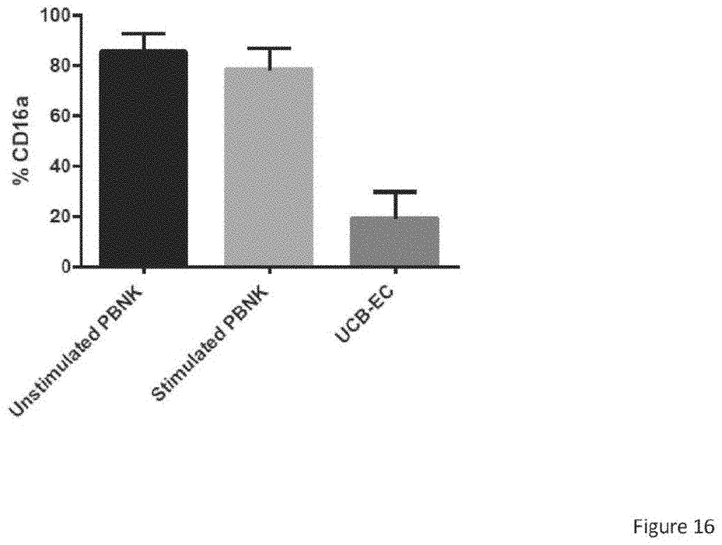

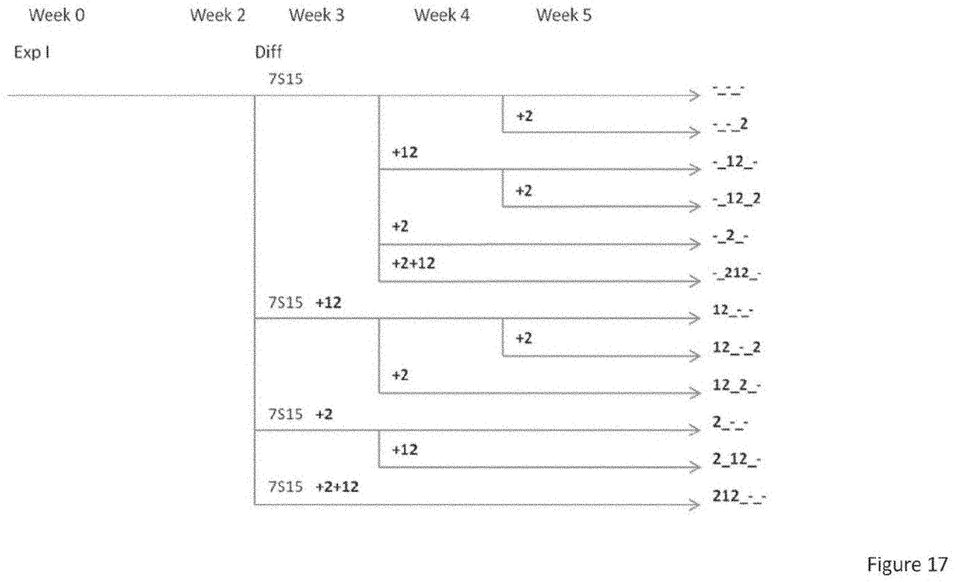

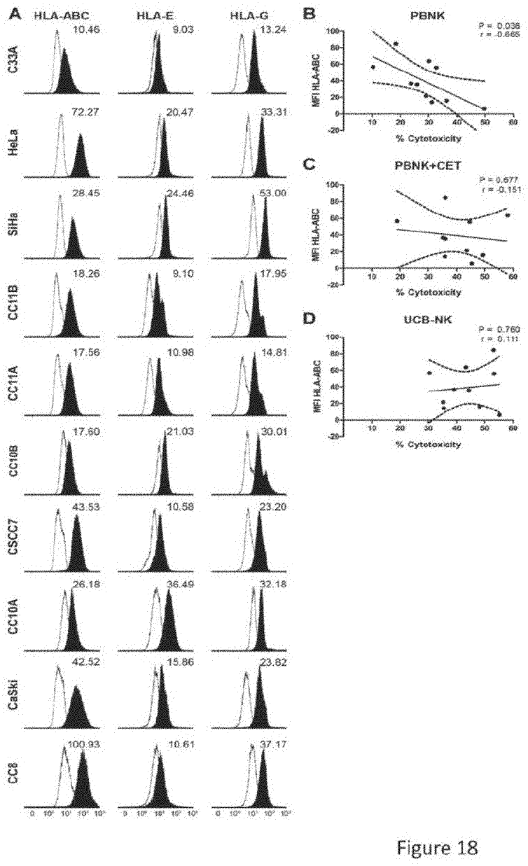

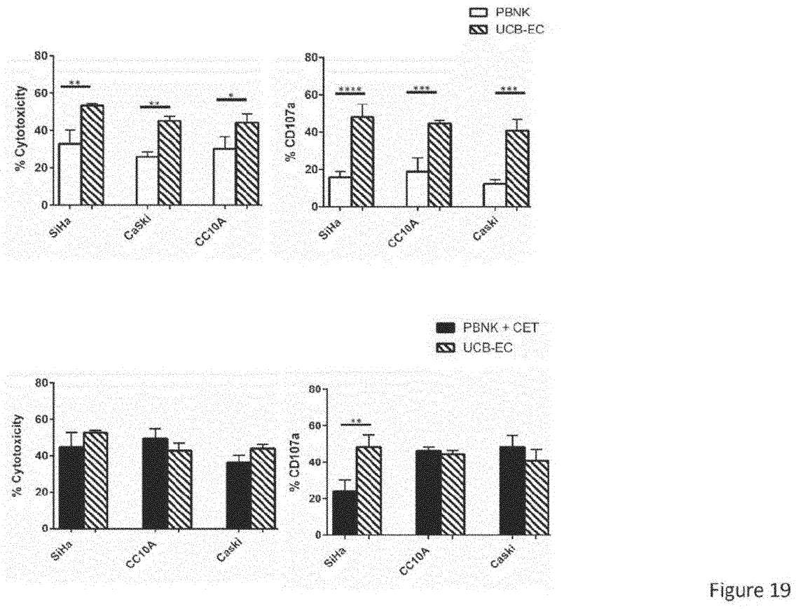

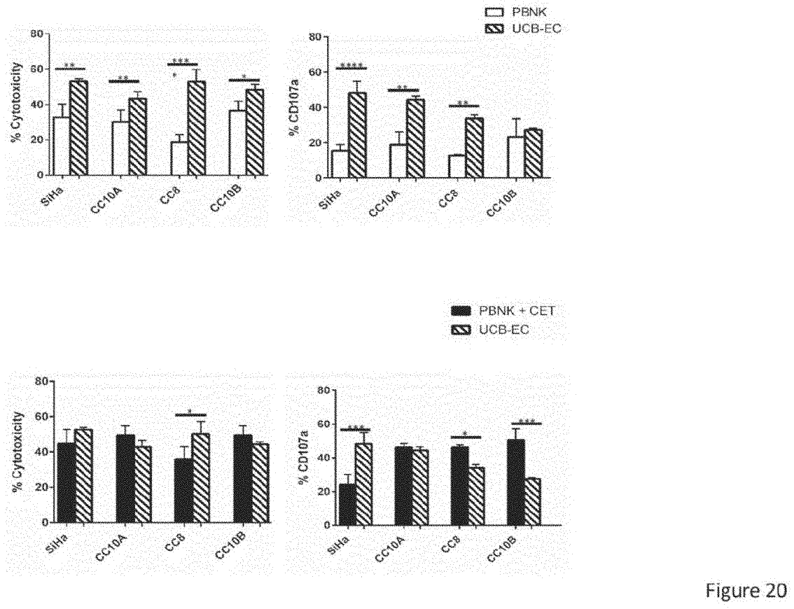

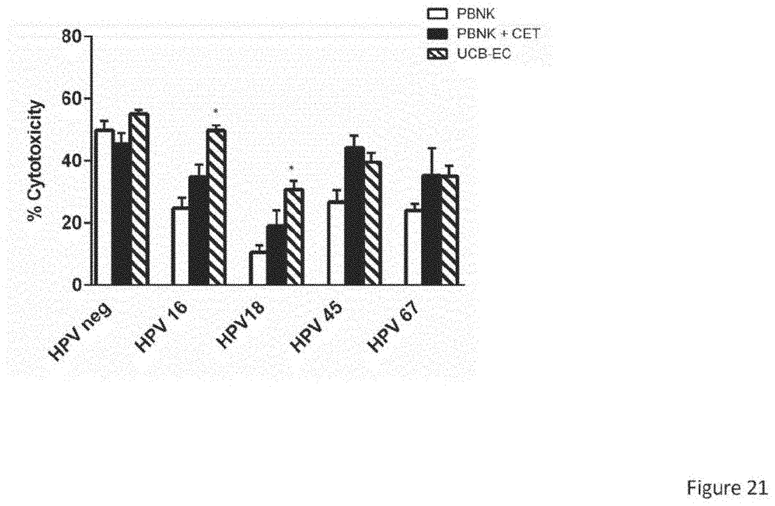

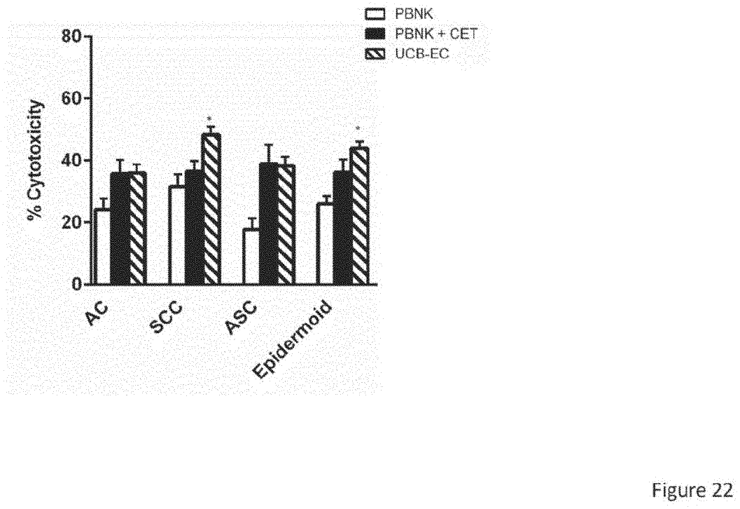

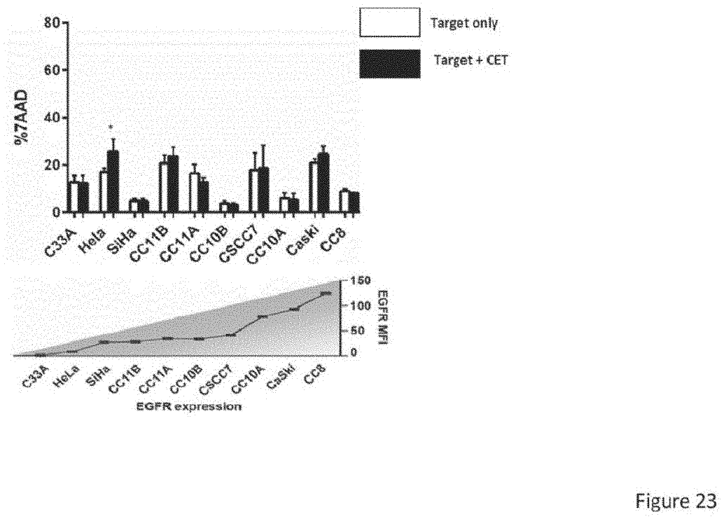

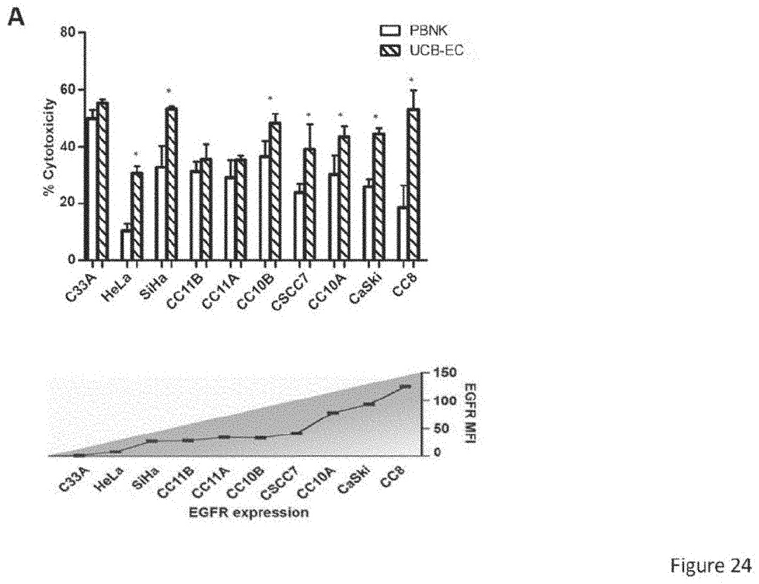

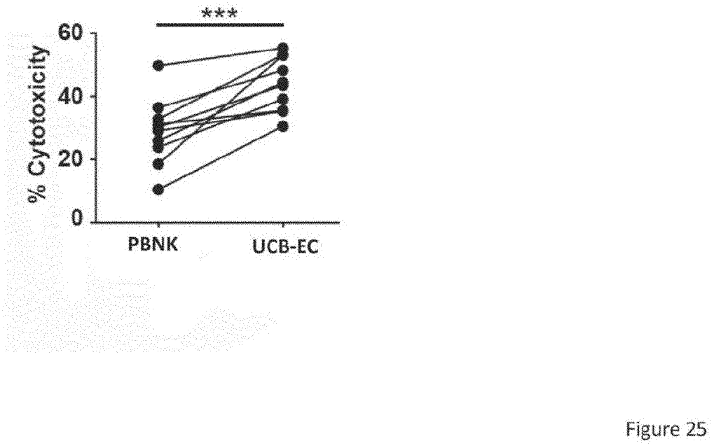

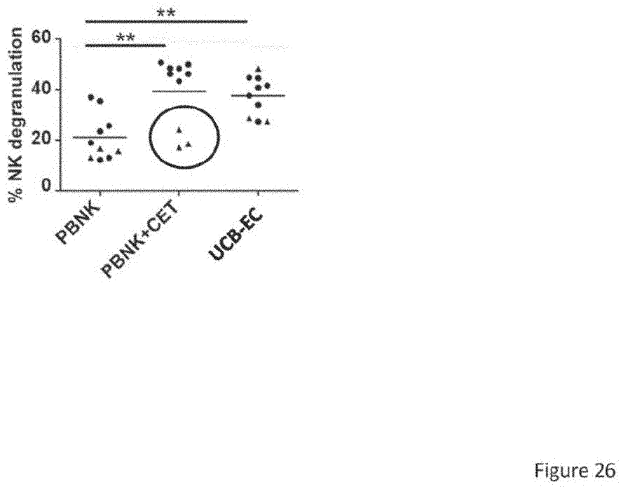

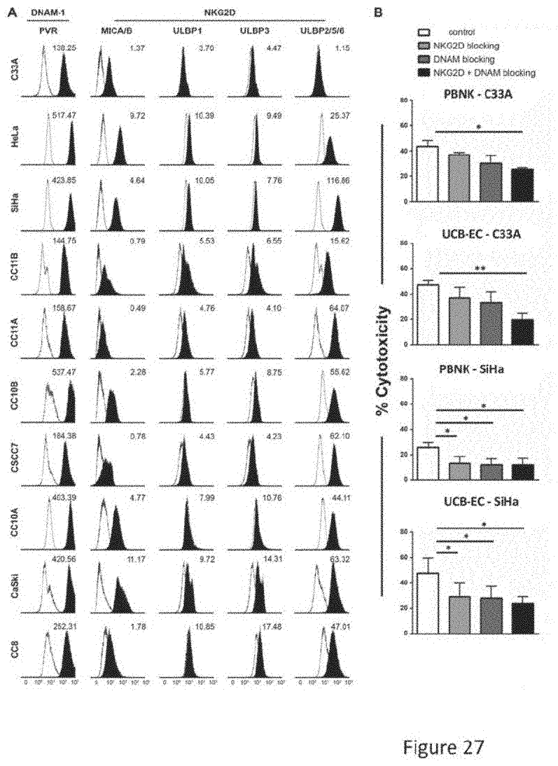

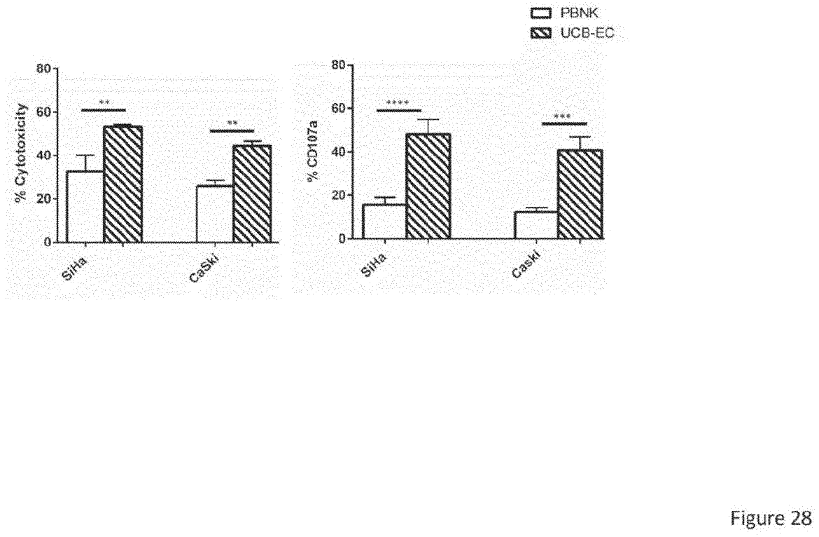

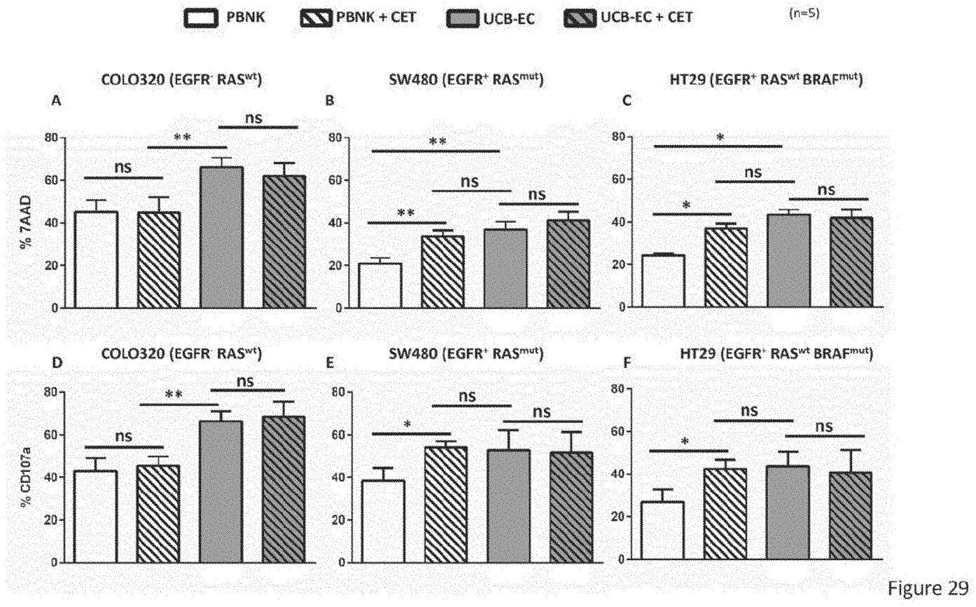

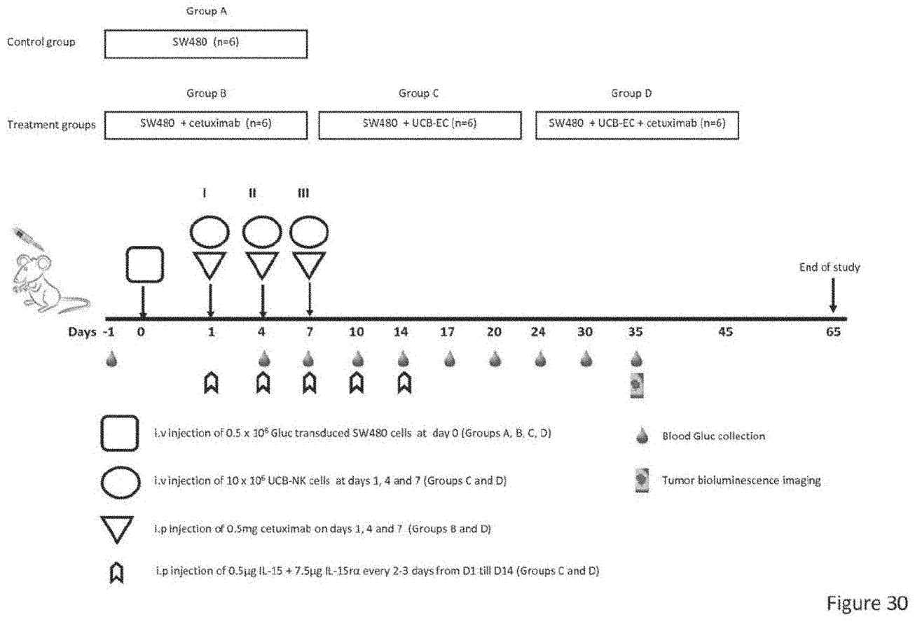

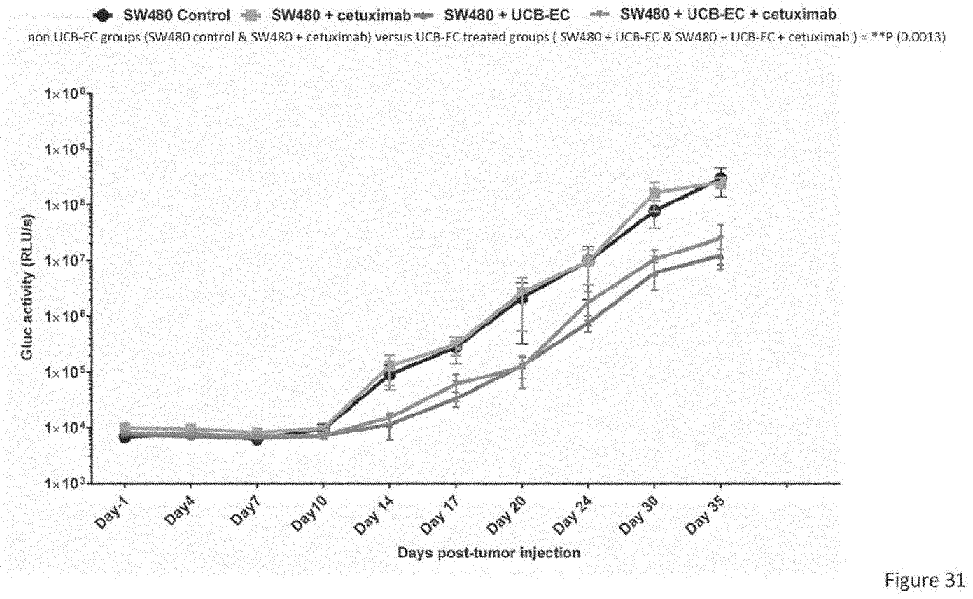

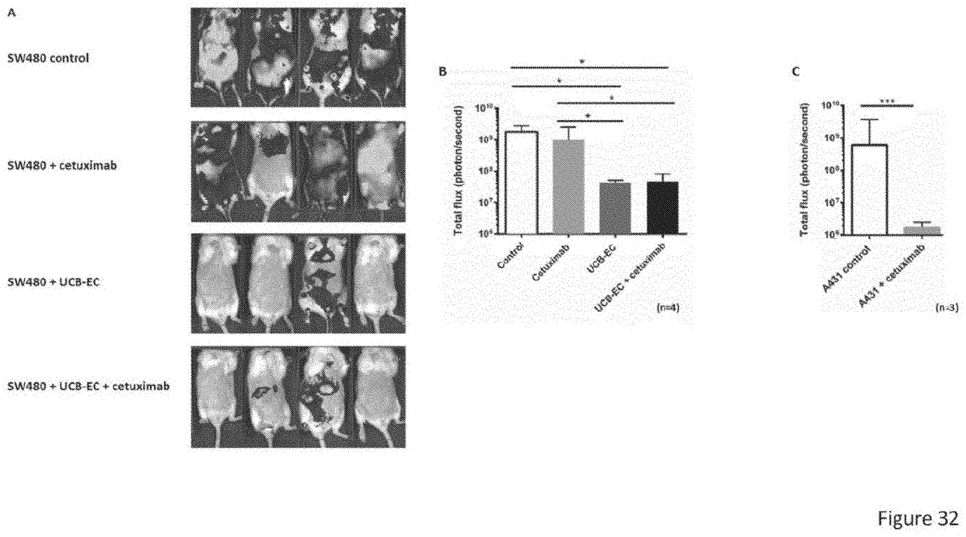

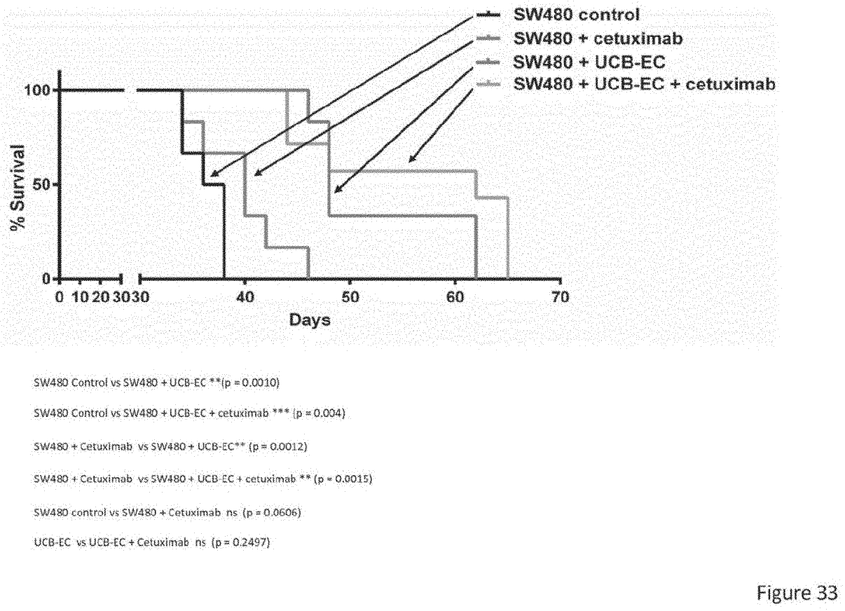

A clinical study as phase I dose escalation trial, using mismatched ex vivo-generated immune effector cells from CD34+ Umbilical Cord Blood (UCB) cells from allogeneic donors (FIG. 1 and Table 1 and 2).

TABLE 1
HLA typing of donor cell product and host

| UPN | HLA type patient* | HLA type donor* |
|---|---|---|
| 1 | A*01 A*03 B*35 B*37 C*04 | A*01 A*03 B*14 B*38 C*08 |
| | C*06 | C*12 |
| 2 | A*11 A*68 B*35 C*03 C*04 | A*01 A*11 B*13 B*35 C*04 |
| | | C*06 |
| 3 | A*02 A*23 B*35 B*44 C*02 | A*02 A*32 B*27 B*44 C*02 |
| | C*04 | C*07 |
| 4 | A*02 A*32 B*15 B*44 C*02 | A*02 A*68 B*15 B*44 C*03 |
| | C*05 | C*07 |
| 5 | A*03 A*74 B*07 B*13 C*06 | A*03 B*07 B*15 C*03 C*07 |
| | C*07 | |
| 6 | A*11 A*66 B*37 B*41 C*06 | A*01 A*11 B*08 B*35 C*04 |
| | C*17 | C*07 |
| 7 | A*02 B*40 B*51 C*03 C*16 | A*01 A*02 B*35 B*51 C*04 |
| | | C*16 |
| 8 | A*01 B*08 C*07 | A*01 B*08 C*07 |
| 9 | A*01 A*24 B*07 B*57 C*06 | A*01 A*68 B*07 C*07 |
| | C*07 | |
| 10 | A*01 A*26 B*18 B*38 C*12 | A*01 A*02 B*18 B*51 C*07 |
| | | C*14 |
| 11 | A*11 A*24 B*37 B*41 C*06 | A*01 A*11 B*35 B*37 C*04 |
| | C*17 | C*06 |
| 12 | A*02 B*07 B*44 C*05 C*07 | A*02 B*27 B*44 C*01 C*05 |

*Matched HLA molecules are in bold and underlined.

The HLA typing was performed in order to identify the differences between donor and patient. Matched genotypes are indicated underlined and in bold.

TABLE 2
KIR typing and matching to HLA ligands

| | | | Missing | Donor | Donor | |
|---|---|---|---|---|---|---|
| | | | ligand | KIR-L | MIR | |
| UPN | MLA type patient* | HLA type donors* | Recipient | mismatch | haplotype | KIR typing |
| 1 | A*01 A*03 B*35 B*37 C*04 C*06 | A*01 A*03 B*14 B*38 C*08 C*12 | C1 | 2DL2/3 | AA | 2DL1/2DL3/2DS4 (A) |
| 2 | A*11 A*68 B*35 C*03 C*04 | A*01 A*11 B*13 B*35 C*04 C*06 | Bw4 | 3DL1 | AB | 2DL1/2DL3/2DS4 (A) |
| | | | | | | en 2DS2/2DL2 (B) |
| 3 | A*02 A*23 B*35 B*44 C*02 C*04 | A*02 A*32 B*27 B*44 C*02 C*07 | C1 | 2DL2/3 | AB | |
| 4 | A*02 A*32 B*15 B*44 C*02 C*05 | A*02 A*68 B*15 B*44 C*03 C*07 | C1 | 2DL2/3 | AA | |
| 5 | A*03 A*74 B*07 B*13 C*06 C*07 | A*03 B*07 B*15 C*03 C*07 | — | — | AB | 2DL1/2DL3/2DS4 (A) en |
| | | | | | | 2DS2/2DS3/2DS4/2DS5 (B) |
| 6 | A*11 A*66 B*37 B*41 C*06 C*17 | A*01 A*11 B*08 B*35 C*04 C*07 | C1 | 2DL2/3 | AA | 2DL1/2DL3/2DS4/3DL1 (A) |
| 7 | A*02 B*40 B*51 C*03 C*16 | A*01 A*02 B*35 B*51 C*04 C*16 | — | — | AB | 2DL1/2DS4/3DL1 (A) en |
| | | | | | | 2DL2/2DS2/2DS3/2DS4 (B) |
| 8 | A*01 B*08 C*07 | A*01 B*08 C*07 | C2, Bw4 | 2DL1, 3DL1 | AA | 2DL1/2DL3/2DS4 (A) |
| 9 | A*01 A*24 B*07 B*57 C*06 C*07 | A*01 A*68 B*07 C*07 | — | — | AA | 2DL1/2DL3/2DS4 (A) |
| 10 | A*01 A*26 B*18 B*38 C*12 | A*01 A*02 B*18 B*51 C*07 C*14 | C2 | 2DL1 | AB | 2DL1/2DL3/2DS4 (A) en |
| | | | | | | 2DS1/2DS3/2DS4/2DS5/ |
| | | | | | | 3DS1 (B) |

Donors and patients are typed for KIR and HLA. The missing HLA ligands for KIR are identified and summarized in the table.
Furthermore the KIR haplotype of all donors was determined, based on the KIR typing.

Donor chimerism was measured by Q-PCR for discriminating DNA polymorphisms. Immune effector cell expansion and phenotype were analyzed by flow cytometry. MRD was evaluated by flow cytometry and molecular techniques. Twelve AML patients (68-76 years) have been included, all in morphologic CR after 2 to 3 standard chemotherapy courses (n=6), or 1 standard chemotherapy course followed by subsequent treatment with hypomethylating agents (azacitidine or decitabine) (n=6). Patients were treated with Cy/Flu and an escalating dose of partially HLA-matched UCB-derived immune effector cells. Four patients had good/intermediate risk, 4 poor risk and 4 very poor risk AML. To date, 9 patients received a composition containing a median of 74% highly activated NCAM positive, CD3 negative immune effector cells, with <1×10⁴/kg CD3+ T cells and <3×10⁵/kg CD19+ B cells. Remaining cells were CD14+ and/or CD15+ monocytic and myelocytic cells. Follow up did not show GVHD or toxicity attributed to the immune effector cells. Two weeks after hematological recovery from consolidation chemotherapy and 6 days before infusion of the ex vivo-generated immune effector cell product, AML patients receive intravenous non-myeloablative immunosuppression consisting of cyclophosphamide (900 mg/m²/day) and fludarabine (30 mg/m²/day) on days −6, −5, −4, −3. This Cy/Flu regimen is administered in an inpatient hospitalized setting. Six days before infusion of the ex vivo-generated immune effector cell product, with the start of non-myeloablative immunosuppression patients receive opportunistic infection prophylaxis consisting of ciproxfloxacin (2 dd 500 mg until recovery of neutropenia), valaciclovir (12 months after start chemotherapy) and co-trimoxazol (1 dd 480 mg) in combination with folic acid (1 dd 5 mg). On day 8 patients receive a single dose of pegfilgrastim (6 mg s.c.) to shorten neutropenia.

The thus immunosuppressed and treated patients receive a 30-minute i.v. infusion of immune effector cells 2 days after the last dose of chemotherapy (day 0). In cohorts of three patients, immune effector cells are infused with an escalating dose of 3×10⁶, 10×10⁶ and 3×10⁷ immune effector cells/kg body weight. Prior to infusion, the patient will receive premedication consisting of acetaminophen 500 mg orally and clemastine 2 mg intravenously. Patients are evaluated including physical examination, toxicity scores and standard blood tests, such as C reactive protein (CRP), hemoglobin (Hb), hematocrit (Ht), complete blood count (CBC), differential, platelets, serum sodium, potassium, calcium, phosphorous, creatinine, bilirubin, albumin, total protein, alkaline phosphatase, gamma glutamyl-transpeptidase (gGt), aspartate aminotransferase (ASAT), alanine transaminase (ALAT), lactate dehydrogenase (LDH), urea). To examine the response to treatment, peripheral blood from patients (pre-study, at 4 hr, day 1, 2, 5, 7, 14, 28 and 56 after immune effector cell infusion) and bone marrow aspirates (pre-study, 7 days, 3 months and 6 months after immune effector cell infusion) are collected.

CD34+ UCB cells are enriched according to JACIE standards of the Stem Cell Laboratory performed in the clean room facility of Laboratory of Hematology. UCB units stored in liquid nitrogen are thawed at 37° C. and resuspended in CliniMACS buffer (Miltenyi Biotec, Bergish Gladbach, Germany) containing 5% HSA, 3.5 mM MgCl2 and 100 U/ml Pulmozyme (clinical grade DNAse) (Roche, Woerden, the Netherlands). All media are clinical-grade and allowed to be used for this purpose. After 30 minutes of incubation, UCB cells are washed and CD34+ cells are enriched using a CliniMACS cell separator after binding with CD34 coupled to immunomagnetic particles according to standard procedures as given by the manufacturer (Miltenyi Biotec, Bergish Gladbach, Germany).

Immune effector cell products are generated from CD34+ UCB cells according to the established protocol⁷². In brief, enriched CD34+ cells are cultured in VueLife™ culture bags (CellGenix) in clinical-grade Glycostem Basal Growth Medium (GBGM) (Clear Cell Technologies, Beernem, Belgium) containing 10% virus-free human serum (Sanquin Bloodbank, Nijmegen), 25□□g/ml low molecular weight heparin (Clivarin©, Flexyx) and GMP-grade recombinant SCF (20 ng/ml), Flt3L (20 ng/ml), IL-7 (20 ng/ml), TPO (20 ng/ml), GM-CSF (10 pg/ml), G-CSF (250 pg/ml) and IL-6 (50 pg/ml) (cytokines are from CellGenix). At day 9, TPO will be replaced with IL-15 (20 ng/ml). From day 10, expanded CD34+ cells will be differentiated into immune effector cells in GBGM medium, 10% human serum, SCF (20 ng/ml), Flt3L (20 ng/ml), IL-7 (20 ng/ml), IL-15 (20 ng/ml), IL-2 (1000 U/ml), GM-CSF (10 pg/ml), G-CSF (250 pg/ml) and IL-6 (50 pg/ml). Cell cultures will be maintained in humidified atmosphere at 37□C with 5% CO2. The final immune effector cell product will be washed and resuspended in infusion buffer (0.9% sodium chloride containing 10% HSA). Cell culturing will be performed according to GMP standards in the clean room facility of Laboratory of Hematology equipped with all necessary devices such as CliniMACS, centrifuges, CO₂ incubators, microscope and automated cell counters.

Ex vivo generated immune effector cell products are tested for the following release criteria:

Microbiological controls: negative for bacterial, fungal and mycoplasma contamination.

Phenotype: Natural cytotoxicity receptors (NCRs), neural cell adhesion molecule (NCAM+), CD94+, CD159a+, CD314+ mature immune effector cells as determined by flow cytometry.

Purity: >70% NCAM+ immune effector cells as determined by flow cytometry.

T cell contamination: <1×10⁴ CD3+ T cells/kg body weight of the patient which is about less than 2×10⁶ total T cells with a patient maximum weight of 200 kg.

B cell contamination: <3×10⁵ CD19+B cells/kg body weight of the patient which is about less than 6×10′ total B cells with a patient maximum weight of 200 kg.

Viability: >70% as determined by 7-AAD exclusion.

Results see table 3.

### Example 2

A group of patients, according example 1, who received intravenous non-myeloablative immunosuppression consisting of cyclophosphamide (900 mg/m²/day) and fludarabine (30 mg/m²/day) on days −6, −5, −4, −3 in an inpatient hospitalized setting.

Prior to infusion and during evaluation of the treatment the following tests are performed:
History, physical examination including vital signs and performance status, toxicity assessment, complete blood count and biochemistries.
Heparinized blood and blood for serum are obtained for immunological studies.
EDTA blood is obtained for AML-MRD analysis.
EDTA blood is obtained for chimerism analysis.
Bone marrow aspiration is performed at 7 days, 3 months and 6 months after cell infusion to evaluate chimerism and the disease status by morphologic, immunophenotypic and molecular analysis. The primary endpoint of this study is to evaluate safety and toxicity of escalating dose infusion of ex vivo-generated immune effector cells following Cy/Flu conditioning. Immune effector cells are infused with an escalating dose of 3×10⁶, 10×10⁶ and 3×10⁷ effector cells/kg body weight. A total of 10 patients are treated in this study (Table 4).

TABLE 3
Characteristics of donor cell product

| | Cell | Cell dose | Purity (% | Viability | CD159 | CD314 | CD337 | CD336 | CD335 | Content | Content |
|---|---|---|---|---|---|---|---|---|---|---|---|
| | dose | infused | NCAM | (% | a (% on | (% on | (% on | (% on | (% on | CD3+ T | CD19+ B |
| UPN | (x10E6) | (x10E6) | positive) | positive) | NCAM) | NA) | NCAM) | NCAM) | NCAM) | cells orma) | cells (xi cr) |
| 1 | 3 | 220 | 75 | 93 | 76 | 83 | 67 | 97 | 72 | 0.10 | 0.00 |
| 2 | 3 | 324 | 81 | 99 | 86 | 92 | 82 | 74 | 73 | 0.00 | 0.00 |
| 3 | 3 | 189 | 71 | 99 | 89 | 98 | 85 | 85 | 85 | 0.00 | 0.00 |
| 4 | 10 | 650 | 58* | 99 | 63 | 100 | 83 | 92 | 79 | 0.00 | 0.31 |
| 5 | 10 | 530 | 74 | 94 | 82 | 99 | 74 | 65 | 63 | 0.00 | 2.54 |
| 6 | 10 | 770 | 74 | 97 | 94 | 99 | 88 | 80 | 82 | 0.00 | 4.75 |
| 7 | 30 | 1693 | 79 | 93 | 72 | na | 80 | 81 | 77 | 0.23 | 4.51 |
| 8 | 17 | 1191 | 65* | 96 | 95 | na | 98 | 81 | 93 | 0.00 | 3.24 |
| 9 | 6 | 510 | 40* | 88 | 90 | 52 | 89 | 68 | 94 | 0.44 | 9.76 |
| 10 | 30 | 2190 | 79 | 51 | 91 | 79 | 98 | 88 | 76 | 0.31 | 0.73 |

*Products are out of specification according to release criteria, but were approved by the hematologist, innnnunologis and QP pharmacist; na = not analyzed. The cell products were analyzed by their composition according specific surface antigen expressions such as CD19, CD3, NCAM and NKG2A, NKG2D, CD334. CD335 as well as CD336 on NCAM positive cells.
na = not analyzed; *marked numbers show values out of specification according release criteria

TABLE 4
Demographic and hematologic characteristics of donor and host

| | | | | | | | | | | Disease |
|---|---|---|---|---|---|---|---|---|---|---|
| | | | | | | | | | | status |
| | | | | | | | Misssing | | | before |
| | Age | | | FAB | Cyto | Molecular | ligand | Donor KIM. | Donor KIR | UCB/NK |
| UPN | (yr) | Sex | WHO type | type | genetics | abberations | Recipient | mismatch | haplotype | infusion |
| 1 | 72 | M | AM. with NPM1 | M2 | 46 XY | NPM1 | C1 | 2DL2/3 | AA | CR1 |
| | | | mutation | | | | | | | |
| 2 | 68 | M | AML M7 | | 46 XY | IDH1 | Bw4 | 3DL1 | AB | CR1 |
| 3 | 73 | F | Therapy related | RAEB-t | Complex | TPS3 | C1 | 2DL2/3 | AB | CR1 |
| | | | AML | | | | | | | |
| 4 | 71 | F | AML | M0 | 46 XX | IDH2, | C1 | 2DL2/3 | AA | CR1 |
| | | | | | | RUNX1 | | | | |
| 5 | 73 | F | AML with MDS- | M1 | 46 XX | IDH2, | — | — | AB | CR1 |
| | | | related features | after | | DNMT3A | | | | |
| | | | | MDS | | | | | | |
| 6 | 76 | M | AML with MDS- | RAEB-t | NE | No known | C1 | 2DL2/3 | AA | CR1 |
| | | | related features | | mutations | | | | | |
| 7 | 75 | F | AML with MDS- | M0 | 46 XX | No known | — | — | AB | CR1 |
| | | | related features | | Trisomy 13 | mutations | | | | |
| 8 | 71 | M | AML | M0/M1 | 46 XY | ASX1.1, | C2, Bw4 | 2DL1, 3DL1 | AA | CR1 |
| | | | | | inversion 12 | RUNX1 | | | | |
| 9 | 71 | M | AML | M5 | 46 XY | FLT3-ITD | | — | AA | CR3 |
| 10 | 73 | M | AML | M5 | 47 XY + 19(8) + | No known | C2 | 2DL1 | AB | CR1 |
| | | | | | 8 + 19(7) | mutations | | | | |

Patients treated with immune effector therapy were given unique patient numbers (UPN). Type of acute myeloid leukemia is
mentioned and known molecular aberration are given. More over the table summarizes the missing KIR ligand on recipients cells
indicating the donor KIR-L mismatch. Further donor KIR haplotype is described and the disease status prior cell infusion is listed. One
patient was in its third clinical remission (CR) prior treatment. All other patients had their first CR.

Toxicity of the immunosuppressive conditioning regimen and cell infusions are separately evaluated. All patients are evaluated intensively for toxicity caused by the conditioning regimen using the CTCAE toxicity criteria and GVHD. No severe toxicities are reported, just a transient cytopenia is monitored due to the conditioning regimen (FIG. 2). Further this treatment shows a reduction in lymphocyte counts till up to day 14 as analysed by using a cell-nucleocounter from the blood samples. Elisa assays on serum levels shows increased IL-15 values after lymphodepletion (all FIG. 3, Table 5).

Both effects are set back to normal levels after 14 days of cell infusion. Moreover, patients are monitored by SNP-PCR according the donor cell chimerism using the blood samples from different time points. All patients show an increase in chimerism after cell infusion up to day 14 and after day 14 donor cell chimerism is not detected in peripheral blood any more (FIG. 4 and Table 4). Moreover, chimerism analysis using the same method in bone marrow samples from day 7/8 after cell infusion clearly shows that the donor cells are detected in patients marrow, likely the site where the leukemia originates (FIG. 4, Table 6).

TABLE 5
IL-15 serum concentration after Cy/Flu immunosuppression and cell infusion

| | UCB-NK | | | | | | | | |
|---|---|---|---|---|---|---|---|---|---|
| | cell dose | IL-IS concentration (pg/ml) in serum after UCB-NK cell Infusion | | | | | | | |
| UPN | (×10\hat{ }6/kg) | day −7 | day 0 | day +1 | +2 days | +6 days | +8 days | +14 days | +28 days |
| 1 | 3 | 21 | 91 | 82 | 80 | 108 | 90 | 66 | 21 |
| 2 | 3 | 6 | 14 | 13 | 15 | 14 | 12 | 13 | 7 |
| 3 | 3 | 6 | 29 | 36 | 32 | 27 | 23 | 20 | 7 |
| 4 | 10 | 12 | 20 | 23 | 22 | N.D. | 30 | 20 | 19 |
| 5 | 10 | 97 | 110 | 115 | 119 | 105 | 92 | 65 | 45 |
| 6 | 10 | 12 | 23 | 31 | 29 | 26 | 29 | 16 | 15 |
| 7 | 30 | 6 | 36 | 32 | 32 | 29 | 31 | 17 | 8 |
| 8 | 17 | 2 | 38 | 40 | 38 | 38 | 51 | 41 | 15 |
| 9 | 6 | 8 | 50 | 45 | 50 | 60 | 51 | 31 | 14 |
| 10 | 30 | | | | | | | | |

The table summarizes the IL-15 levels analyzed after conditioning immunosuppression and cell infusion in pg/ml.
N.D. = not determined

TABLE 6
Chimerism of donor cells in whole blood and bone marrow

| | UCS | | | | | | | | Donor thlmerism |
|---|---|---|---|---|---|---|---|---|---|
| | cell dose | Donor chimerism (%) in WBC after UCB cell Infusion | | | | | | | in BM aspirate (%) |
| UPN | (×10\hat{ }6/kg) | +4 hour | +1 day | +2 days | +5/6 days | +7/8 days | +14 days | +28 days | +7/8 days |
| 1 | 3 | 0.00 | 0.06 | 0.05 | 0.26 | 0.35 | 0,00 | 0.00 | NE |
| 2 | 3 | Oil | 0.02 | 0.03 | NE | 0.13 | 0.03 | 0.00 | 0.06 |
| 3 | 3 | 0.04 | 0.06 | NE | 1.04 | 0.10 | 0.00 | 0.00 | 0.00 |
| 4 | 10 | NE | NE | 0.13 | 2.09 | 0.36 | 0.00 | 0.00 | 0.08 |
| 5 | 10 | 0.33 | 0.06 | NE | 7.23 | 21* | 0.00 | 0.00 | 3.50 |
| 6 | 10 | 0.49 | 0.18 | NE | 2.26 | 0.43 | 0.00 | 0.00 | 0.25 |
| 7 | 30 | NE | NE | 12.94 | NE | 3.25 | 0.00 | 0.00 | 1.19 |
| 8 | 17 | 2.33 | — | 1.58 | — | 9.35 | 0.00 | 0.00 | 2.05 |
| 9 | 6 | NE | — | 6.09 | — | 0.59 | 0.00 | 0.00 | 0.60 |
| 10 | 30 | — | | | | | | | |

*Chimerism dtermined by flow cytometry using anti HLA-B13 discriminating antibody;
NE = not evaluable due to low DNA amount
Donor chimerism in % of total whole blood cells (WBC) or bone marrow (BM) is given. Analysis was done by single nucleotide polymorphism (SNP) Q-PCR analysis. Number marked with (*) has been determined by flow cytometry using anti-HLA-B13 antibody.
NE = not evaluable due to low DNA amount

### Example 3

Patients selected and conditioned in the study as described in example 1 and 2, which receive the treatment and where infused effector cells are traced by flow cytometry using monoclonal antibodies such as anti-NCAM and CD3 (FIG. 5) as used in previous studies⁷⁷. As a temporary repopulation and persistence of UCB-derived immune effector cells could be detected in peripheral blood of patients, between days 1 and 8 post infusion, which was associated with increased IL-15 plasma levels observed in most patients. Interestingly, donor chimerism increased with higher doses of infused UCB-derived immune effector cells, and donor chimerism up to 3.5% was found in bone marrow (BM) at day 7/8. Further UCB-immune effector cell maturation in vivo was observed by acquisition of CD16 and KIRs, while expression of activating receptors was sustained. Of the 9 treated patients so far, 5 (56%) are still in CR after 43, 35, 31, 5 and 4 months, whereas 4 patients relapsed after 5, 6 (2 pts) and 15 months. Despite morphologic CR during azacitidine treatment, residual disease of 6-7% with a leukemia-associated phenotype could be detected by flow cytometry before immune effector cell infusion in BM of two patients. In both patients MRD was reduced to less than 0.05% at 90 days after UCB-derived immune effector cell therapy following Flu/Cy conditioning. These results show that GMP-compliant UCB-derived immune effector cells containing up to 30×10⁶ immune effector cells/kg body weight can be safely infused in non-transplant eligible AML patients following immunosuppressive chemotherapy. Moreover, some of those patients had detectable minimal residual disease and such potential clone of leukemic blasts were described by Leukemic associated phenotype (LAP) CD45+/CD34+/CD117−/CD133+ as analysed by flowcytometry⁷⁸. After immunotherapy using the cell product of this invention, a reduction in leukemic blast count from 6.7% towards an undetectable limit <0.01% could be observed (FIG. 6). In UPN8 a potential clone of leukemic blasts was described by Leukemic associated phenotype (LAP) CD45+/CD34+/CD7+/CD133+. After immunotherapy using the cell product of this invention, a reduction in leukemic blast count from 6.3% towards and a nearly undetectable limit of 0.02% could be observed (FIG. 7). Furthermore these patients were followed after the cell therapy treatment and monitored for relapse and survival. A superior survival was observed for the treated patient group compared to the historical survival of elderly AML patients (FIG. 8, Table 7). Also the relapse rate show a benefit for these patient as only slightly over 50% of the patients have got a relapse (FIG. 9, Table 7).

TABLE 7
Risk group qualification and patient follow up

| | Age | | | | | Follow up |
|---|---|---|---|---|---|---|
| UPN | (yr) | Sex | WHO type | FAB type | Risk group | (Days) |
| 1 | 72 | M | AML with NPM1 | M2 | Good/inter- | 1391 |
| | | | mutation | | mediate risk | |
| 2 | 68 | M | AML | M7 | Intermediate | 1133 |
| | | | | | risk | |
| 3 | 73 | F | Therapy related AML | RAEB-t | Very poor risk | Relapse at day +136; |
| | | | | | | died at day +421 |
| 4 | 70 | F | AML | M0 | Intermediate | 1028 |
| | | | | | risk | |
| 5 | 72 | F | AML with MDS- | M1 after | Poor risk | Relapse at day +168; |
| | | | related features | MDS | | died at day +679 |
| 6 | 76 | M | AML with MDS- | RAEB-t | Poor risk | Relapse at day +438; |
| | | | related features | | | died at day +452 |
| 7 | 75 | F | AML with MDS- | M0 | Poor risk | Relapse at day +194; |
| | | | related features | | | died at day +306 |
| 8 | 71 | M | AML | M0/M1 | Very poor risk | Relapse at day +181; |
| | | | | | | follow up 237 |
| 9 | 71 | M | AML | M5 | Very poor risk | 216 |
| 10 | 73 | M | AML | M5 | Poor risk | 62 |

numbers are used for the calculation of overall survival and progression free survival.

### Example 4

Antitumor efficacy of the effector cells compared with PBNK cells using flow cytometry based cytotoxicity and degranulation assays according to the basic protocols as described before⁷⁶.

More in detail, here colon cancer cell lines COLO320 (EGFR−, RAS^{wt}), SW480 (EGFR+, RAS^{mut}) and HT29 (EGFR−, RAS^{wt}, BRAF^{mut}) where anti-EGFR therapy can be expected to be ineffective are subjected to a comparison of cord blood generated effector cells (UCB-EC) and peripheral blood activated natural killer cells (PBNK) killing. From the results it is evident that both RAS^{wt} & ^{mut} colon cancer cells are more sensitive to UCB-EC killing than PBNK cells. Another important aspect of UCB-EC cells is that they overcome HLA-E resistance, for instance SW480 cells have high HLA-E expression often translating into superior killing than PBNK cells. These data show that UCB-EC cells have the potential to improve colon cancer therapy efficacy even in situations where tumors carry RASmut or are EGFR−.

Cell Lines

Cell lines A431 (epidermoid carcinoma), Colo320, SW480 (colorectal carcinoma) and Hela, Siha, Caski, C33A, CSCC7, CC8, CC10A, CC10B, CC11A, CC11B (cervical carcinoma) are obtained from ATCC or cell stock from patient derived cell lines (Leiden university) and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator.

Isolation and Activation of Peripheral Blood NK Cells from Whole Blood Specimens

Whole blood from healthy volunteers is collected with written informed consent. Mononuclear cells (MNCs) are isolated using Lymphoprep™ (STEMCELL Technologies, The Netherlands) density gradient centrifugation PBNK cells are isolated from MNCs using a MACS Human NK cell isolation kit (Miltenyi Biotech, Bergisch Gladbach, Germany) according to the manufacturer's instructions. The cell number and purity of the isolated NK cell fraction are analyzed by flow cytometry. Isolated NK cells are activated overnight with 1000U/ml IL-2 (Proleukin®; Chiron, München, Germany) and 10 ng/ml IL-15 (CellGenix) for use in cytotoxicity assays. NK cell purity and viability are checked using CD3 PE, 7AAD (BD Biosciences), CD56 APC Vio 770, and CD16 APC (Miltenyi Biotech). The preliminary parameters noted before and after activation are NK purity (CD56+%, 83 ±9% & 82±9%), NK CD16% 88 ±10% & 85 ±11%) and NK viability (91 ±3% & 86 ±2%) respectively.

UCB-EC Cell Cultures

Ex Vivo Expansion of CD34-Positive Progenitor Cells

CD34+ UCB cells (between 1×10⁴ and 3×10⁵ per ml) are plated into 24-well tissue culture plates (Corning Incorporated, Corning, N.Y.) in GBGM supplemented with 10% human serum (HS; Sanquin Bloodbank, Nijmegen, The Netherlands), 20 ng/mL of SCF, Flt-3L, TPO, IL-7 (all CellGenix). From Day 9-14, TPO is replaced with 20 ng/mL IL-15 (CellGenix) in the expansion cultures. During the first 14 days of culture, low molecular weight heparin (LMWH) (Clivarin®; Abbott, Wiesbaden, Germany) is added to the expansion medium in a final concentration of 25 μg/ml and a low-dose cytokine cocktail consisting of 10 pg/ml GM-CSF, 250 pg/ml G-CSF, (Stemcell Technologies) and 50 pg/ml IL-6 (CellGenix, Freiburg, Germany). Cell cultures are refreshed with new medium every 2-3 days. Cultures are maintained in a 37° C., 95% humidity, 5% CO2 incubator.

Differentiation of Ex Vivo Expanded CD34-Positive Cells into UCB-EC Cells

Expanded CD34+ UCB cells are differentiated and further expanded using effector cell differentiation medium. This medium consists of the same basal medium as used for the CD34 expansion step supplemented with 2% HS, the low-dose cytokine cocktail (as previously mentioned) and a new high-dose cytokine cocktail consisting of 20 ng/ml of IL-7, SCF, IL-15 (CellGenix) and 1000 U/ml IL-2 (Proleukin®; Chiron, München, Germany) is added to the differentiation medium. Medium is refreshed twice a week from day 14 onwards.

Flow Cytometry

Flow cytometry analysis is performed on a BD LSR FORTESSA X-20 (BD Biosciences). Cell numbers and expression of cell-surface markers are determined by flow cytometry. The cell numbers and the population of live cells is determined by gating on CD45+ cells based on forward scatter (FSC) and side scatter (SSC). For analysis of phenotype, the cells were gated only on FSC/SSC and further analyzed for the specific antigen of interest. Cells were incubated with the appropriate concentration of antibodies for 30 min at 4° C. After washing, cells are suspended in FACS buffer.

Flow Cytometry-Based Cytotoxicity and Degranulation Studies

Flow cytometry is used for the read-out of cytotoxicity assays. Target cells are labeled with 5 μM pacific blue succimidyl ester (PBSE; Molecular Probes Europe, Leiden, The Netherlands) in a concentration of 1×10⁷ cells per ml for 10 min at 37° C. The reaction is terminated by adding an equal volume of FCS, followed by incubation at room temperature for 2 min after which stained cells are washed twice with 5 ml DMEM/10% FCS. After washing, cells are suspended in DMEM/10% FCS to a final concentration of 5×10⁵/ml. PBNK and UCB-EC cells are washed with PBS and suspended in Glycostem Basal Growth Medium (GBGM)+2% FCS to a final concentration of 5×10⁵/ml. Target cells are co-cultured with effector cells at an E:T ratio of 1:1 in a total volume of 250 μl in 96-wells flat-bottom plates (5×10⁴ targets in 100 μl of DMEM+10% FCS incubated with 5×10⁴ effectors in 100 μl of GBGM+2% FCS, further supplemented with 25 μl of GBGM+2% FCS and DMEM+10% FCS medium). PBNK cells, UCB-EC cells and target cells alone are plated out in triplicate as controls. To measure degranulation by PBNK and UCB-EC cells, anti-CD107a PE (Miltenyi Biotech, Germany) is added in 1:20 dilution to the wells. After incubation for 4h at 37° C., 75 μl supernatant is collected and stored at −20° C. for analysis of cytokine production. Cells in the remaining volume are harvested and stained with 7AAD (1:20). Degranulation of PBNK and UCB-EC cells is measured by detecting cell surface expression of CD107a. After 4 hrs of incubation at 37° C., CD56 APC Vio 770 (1:25) and CD16 APC (1:25) (Miltenyi Biotech, Germany) are added to the co-cultures and NK CD107a degranulation is measured for CD56+ PBNK and UCB-EC cells.

Statistical Analysis

Statistical analysis is performed using Graph Pad Prism software. Differences between conditions are determined using two way Anova with multiple comparisons between column means. Results from cytotoxicity experiments are described as mean±standard deviation of the mean (SD). A p-value of <0.05 is considered statistically significant.

The results show significant better superior killing of UCB-EC versus PBNK on Epidermoid carcinoma (FIG. 10) as well as on colon cancer (FIG. 11) and cervical cancer (FIG. 12).

### Example 5

Antitumor efficacy of the effector cells is tested using flow cytometry based cytotoxicity and degranulation assays. Myeloid cancer cells K562 (CML), U266 (multiple myeloma), CCRF-CEM (T cell ALL), MOLT 4 (T cell ALL) and solid tumor cells like MIA PaCa-2 (ductual carcinoma) and NCI-H82 (small lung cell carcinoma) are used for killing assays with cord blood effector cells (UCB-EC) according the same methods as described in example 4.

Cell Lines

Cell lines are cultured in IMDM or Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator.

UCB-EC Cell Cultures

Ex Vivo Expansion of CD34-Positive Progenitor Cells

More specific here, CD34+UCB cells are plated into 24-well tissue culture plates (Corning Incorporated, Corning, N.Y.) in Glycostem Basal Growth Medium (GBGM) (Clear Cell Technologies, Beernem, Belgium) supplemented with 10% human serum (HS; Sanquin Bloodbank, Nijmegen, The Netherlands), 20 ng/mL of SCF, Flt-3L, TPO, IL-7 (all CellGenix). From Day 9-14, TPO is replaced with 20 ng/mL IL-15 (CellGenix) in the expansion cultures. During the first 14 days of culture, low molecular weight heparin (LMWH) (Clivarin®; Abbott, Wiesbaden, Germany) is added to the expansion medium in a final concentration of 25 pg/ml and a low-dose cytokine cocktail consisting of 10 pg/ml GM-CSF, 250 pg/ml G-CSF, (Stemcell Technologies) and 50 pg/ml IL-6 (CellGenix, Freiburg, Germany). Cell cultures are refreshed with new medium every 2-3 days. Cultures are maintained in a 37° C., 95% humidity, 5% CO2 incubator.

Differentiation of Ex Vivo Expanded CD34-Positive Cells into UCB-EC Cells

Expanded CD34+ UCB cells are differentiated and further expanded using effector cell differentiation medium. This medium consists of the same basal medium as used for the CD34 expansion step supplemented with 2% HS, the low-dose cytokine cocktail (as previously mentioned) and a new high-dose cytokine cocktail consisting of 20 ng/ml of IL-7, SCF, IL-15 (CellGenix) and 1000 U/ml IL-2 (Proleukin®; Chiron, München, Germany) is added to the differentiation medium. Medium is refreshed twice a week from day 14 onwards.

Flow Cytometry

Flow cytometry analysis is done on a FACS Canto (BD Biosciences). Cell numbers and expression of cell-surface markers are determined by flow cytometry. The cell numbers and the population of live cells is determined by gating on CD45+ cells based on forward scatter (FSC) and side scatter (SSC). For analysis of phenotype, the cells are gated only on FSC/SSC and further analyzed for the specific antigen of interest. Cells are incubated with the appropriate concentration of antibodies for 30 min at 4° C. After washing, cells are suspended in FACS buffer.

Flow Cytometry-Based Cytotoxicity and Degranulation Studies

Flow cytometry is used for the read-out of cytotoxicity assays. Target cells are labeled with 5 μM pacific blue succimidyl ester (PBSE; Molecular Probes Europe, Leiden, The Netherlands) in a concentration of 1×10⁷ cells per ml for 10 min at 37° C. The reaction is terminated by adding an equal volume of FCS, followed by incubation at room temperature for 2 min after which stained cells are washed twice with 5 ml DMEM/10% FCS. After washing, cells are suspended in DMEM/10% FCS to a final concentration of 5×10⁵/ml. PBNK and UCB-EC cells are washed with PBS and suspended in Glycostem Basal Growth Medium (GBGM)+2% FCS to a final concentration of 5×10⁵/ml. Target cells are co-cultured with effector cells at an E:T ratio of 1:1 or 50:1 in a total volume of 250 μl in 96-wells flat-bottom plates (5×10⁴ targets in 100 μl of DMEM+10% FCS incubated with 5×10⁴ effectors in 100 μl of GBGM+2% FCS, further supplemented with 25 μl of GBGM+2% FCS and DMEM+10% FCS medium). PBNK cells, UCB-EC cells and target cells alone are plated out in triplicate as controls. To measure degranulation by PBNK and UCB-EC cells, anti-CD107a PE (Miltenyi Biotech, Germany) is added in 1:20 dilution to the wells. After incubation for 4h at 37° C., 75 μl supernatant was collected and stored at −20° C. for analysis of cytokine production. Cells in the remaining volume are harvested and stained with 7AAD (1:20). Degranulation of PBNK and UCB-EC cells is measured by detecting cell surface expression of CD107a. After 4 hrs of incubation at 37° C., CD56 APC Vio 770 (1:25) and CD16 APC (1:25) (Miltenyi Biotech, Germany) are added to the co-cultures and NK CD107a degranulation was measured for CD56+PBNK and UCB-EC cells.

The results show high killing of different myeloid (FIG. 13) and solid tumor cell lines (FIG. 14) by UCB-EC.

### Example 6

An Experiment, where the expression levels of FcRγIIIa (CD16a) is analyzed using standard flowcytometry. All cell products used in the clinical study as described in Example 1 and 2 show a low FcRγIIIa expression (FIG. 15). Further immune effector cells generated from cord blood are compared with natural killer cells (PBNK). Even after overnight IL-2 stimulation 1000U/ml GBGM medium PBNK remain a high CD16 expression (FIG. 16). In a first step, a new serum free medium was developed, that could be used for the expansion of progenitor cells as well as for the differentiation of functional EC cells. Specifically, the medium is formulated with only human or recombinant human proteins to enable the translation towards clinical or pharmaceutical production. Systematic refinement of a supplemental cytokine cocktail in combination with clinical grade heparin leads to a highly efficient cell culture protocol. NCAM positive, CD3 negative UCB-EC Cell Product could be routinely generated at laboratory scale from freshly isolated CD34+UCB cells with a mean expansion of >15,000 fold and a nearly 100% purity, devoid of any T and B cells. A relatively high percentage of this NCAM positive, CD3 negative EC cell population expressed the inhibitory CD94/ECG2A complex (50-90%), while only an intermediate subset was low positive for CD16. Furthermore, UCB-EC Cell Product contained about 5-10% EC cell subsets expressing KIR receptors specific for both HLA-Cw group 2 alleles (KIR2DL1/DS1), HLA-Cw group 1 alleles (KIR2DL2/DS2) and HLA-Bw alleles (KIR3DL1/DS1). Moreover, UCB-EC Cell Product expresses several cytokine receptor chains for IL-2 (CD25; IL-2R), SCF (CD117), IL-7 (CD127; IL-7R) and IL-15 (CD122; IL-15R) as well as chemokine receptors (e.g. CXCR4, CXCR3) which might be important for in vivo expansion and migration of the infused EC cells. These data illustrate that the final NCAM positive, CD3 negative UCB-EC Cell Product displays an activated phenotype regarding the expression of activating and inhibitory EC cell receptors as well as cytokine receptors important for cell survival.

The novel cytokine and heparin based culture protocol for ex vivo expansion of EC cells from umbilical cord blood (UCB) hematopoietic stem cells, was translated into a fully closed, large-scale, cell culture bioprocess⁷². By passing hurdles like the optimization of CD34+ selection from cryopreserved “off-the-shelf” UCB products using a closed process, various bioreactor systems have been tested to develop and optimize a completely closed cell culture process to generate large numbers of EC cells. In order to utilize UCB-EC Cell Product for adoptive immunotherapy in poor-prognosis AML patients, the method was adapted into a closed-system bioprocess for production of UCB-EC Cell Product batches under GMP conditions. Large-scale experiments using gas-permeable culture bags first demonstrated that the two-step expansion and differentiation protocol reproducibly generates NCAM positive, CD3 negative UCB-EC Cell Product cells from UCB-derived CD34+ cells enriched by the CliniMACS cell separator (Miltenyi Biotec) with an average purity of 70%. Contaminating cells in those cultures represented mature myeloid cells. The numbers of contaminating T and B cells were very low (<0.01% CD3+ cells and <0.01% CD19+ cells, respectively). By further upscaling of the EC cell expansion step into the WAVE Bioreactor™ system (GE Healthcare) between 1-10×10⁹ EC cells from 1-10×10⁶ UCB-derived CD34+ cells could be generated and also the purity could be increased to more than 90% NCAM+ CD3− EC cells. Extensive product release testing and downstream processing ensure a safe and well-controlled release of the EC cell immunotherapy product. UCB-EC cell product was further tested for sterility, viability and the absence of endotoxins and remaining cytokines from the culture medium, with all four test runs passing the release criteria. Moreover extensive karyotyping tests have shown no abnormalities and also the cell recovery of more than 80% after washing showed an acceptable result. These results demonstrate that large numbers of UCB-EC Cell Product for adoptive immunotherapy can be produced in closed, large-scale bioreactors for the use in clinical trials.

### Example 7

CML K562 and AML cell lines KG1a and THP-1 (LGC Standards, Wesel, Germany) were thawed at 37° C. and resuspended in Iscove's modified Dulbecco's medium (IMDM; Invitrogen, Carlsbad Calif., USA) with 10% fetal calf serum (FCS; Integro, Zaandam, the Netherlands). Cultures were placed in T25 or T75 flasks (Greiner Bio-One GmbH, Frickenhausen, Germany) in IMDM supplemented with 50 U/ml penicillin, 50 μg/ml streptomycin (PS, MP Biomedicals, Solon, USA) and 10% FCS at 37° C. and 5% CO₂. Media was refreshed every 3 or 4 days to place the cells at a density between 2*10⁵ and 3*10⁵ cells/mL.

Mononuclear cells were selected from umbilical cord blood (UCB; cord blood bank Radboud University Nijmegen Medical Center (RUNMC)) using gradient with Ficoll-Paque 1077 Plus (GE Healthcare) according to the manufacturers protocol. After red blood cells were lysed by incubating for 10 min with ery-lysis buffer, the white blood cells were spun down and washed with phosphate buffered saline (PBS) and checked for CD34+ cells by staining with 1 μl CD34-PC7 (581, Beckman Coulter, Fullerton, USA). CD34+ cells were selected using anti-CD34 immunomagnetic bead separation (Miltenyi Biotech, Bergisch Gladbach, Germany) according to the manufacturers protocol. CD34− and CD34+ cells were separately resuspended in 1:1 Human Serum (HS; Sanquin Bloedbank, Nijmegen) and Glycostem Basal Growth Medium for Cord Blood (GBGM, Clear Cell Technologies, Beernem, Belgium) containing 7% DMSO and stored in liquid nitrogen.

CD34+ UCB cells were thawed at 37° C. and resuspended in HS containing 2.5 mM MgCl₂ and 100 μl DNAse. After 10 min of incubation, the hematopoietic progenitor stem cells were washed and plated into 24-well (Corning Incorporated, Corning, N.Y.) and expanded for the first 14 days. GBGM was supplemented with 10% HS and a low-dose cytokine cocktail consisting of 10 pg/ml GM-CSF, 250 pg/ml G-CSF (Stemcell Technologies) and 50 pg/ml IL-6 (CellGenix, Freiburg, Germany). Also a high-dose cytokine cocktail was added consisting of 27 ng/ml SCF, 25 ng/ml Flt3L, 25 ng/ml TPO, 25 ng/ml IL-7 (all CellGenix) and 25 μg/ml low molecular weight heparin (LMWH; Clivarin®; Abbott, Wiesbaden, Germany). Cell cultures were refreshed every 2-3 days and maintained at 37° C., 95% humidity and 5% CO2.

From day 14 onward the expanded CD34+UCB cells were further expanded and differentiated using UCB-EC cell differentiation medium consisting of GBGM-CB®, 10% HS and low-dose cytokine cocktail as previously described. The high-dose cytokine cocktail was varied with IL-15, IL-2, IL-7, IL-12 and SCF (all CellGenix)(Table 8). The cell density was checked every 3-4 days and adjusted to ˜1,5*10⁶ cells/ml by adding or refreshing differentiation medium. Again cultures were maintained in a 37° C., 95% humidity and 5% CO₂ incubator.

TABLE 8
High dose cytokine combinations used during differentiation for
optimizing ex vivo expansion of UCB-EC cells. UCB-EC cultures
were supplemented during the differentiation period with various
high dose cytokine combinations as implicated in the table.
IL-15 was used in all cultures and all possible combinations
of SCF, IL-2 and IL-7 were added to analyze the effect of
these cytokines on the UCB-EC cell expansion,
differentiation and functionality.

| | | | |
|---|---|---|---|
| 1. IL-15 | 3. IL-15 IL- | 5. IL-15 IL- | 7. IL-15 IL- |
| 2. IL-15 | 4. IL-15 IL- | 6. IL-15 IL- | 8. IL-15 IL- |

UCB-EC Cell Product Assessment

The viable UCB-EC cell product was counted every 3 to 4 days using 50 μl culture (at 0.5-2.5*10⁶ cells/ml) and staining the cells with 1.5 μl CD45-ECD (J33, Beckman Coulter, Fullerton, USA) and 1 μl NCAM-PC7 (N901, Beckman Coulter, Fullerton, USA) in a volume of 100 μl. After 15 min of incubation at 4° C. with these antibodies 7-Aminoactinomycin D (7-AAD, Sigma St. Louis, USA) was added to exclude any apoptotic cells. Also the maturation of the product was checked every week by staining during expansion with antibody mix 1 and during differentiation with mix 2 and 3 (Table 9), both ˜150.000 cells in a volume of 25 μl for 15 min at 4° C. All stainings were measured by flowcytometry on the FC500 cytometer (FC500, Beckman Coulter, Fullerton, USA). Additional staining mixes are mentioned in the results.

TABLE 9
Antibodies used during phenotyping of the UCB-EC Cells
(~150.000) were incubated with indicated amount
of antibody in a volume of 25 μl for 15 min at 4° C.

| Mix 1 | | Mix 2 | | Mix 3 | |
|---|---|---|---|---|---|
| CD7 | 1:25, | CD33 | 1:25, Fitc, | CD11c | 1:12.5, |
| | Fitc,8H8.1, | | D3 H L60,251, | | Fitc, KB90, |
| | Beckman | | Beckman | | Dako |
| | Coulter | | Coulter | | |
| CD133 | 1:25, PE, | NKG2a | 1:25, PE, | CD11a | 1:10, PE, |
| | AC133, | | Z199, Beckman | | 25.3, Beckman |
| | Miltenyi | | Coulter | | Coulter |
| CD45 | 1:25, ECD, | CD3 | 1:25, | CD14 | 1:40, |
| | J33, | | ECD, UCHT1, | | ECD, RMO52, |
| | Beckman | | Beckman | | Beckman |
| | Coulter | | Coulter | | Coulter |
| CD117 | 1:25, PC5, | CD117 | 1:25, | CD56 | 1:40, |
| | 104D2D1, | | PC5, 104D2D1 | | PC5, N901 |
| | Beckman | | Beckman | | Beckman |
| | Coulter | | Coulter | | Coulter |
| CD34 | 1:25, PC7, | CD117 | 1:25, | CD11b | 1:50, |
| | 581, | | PC7, N901 | | PC7, Bear1, |
| | Beckman | | Beckman | | Beckman |
| | Coulter | | Coulter | | Coulter |

Information indicates: dilution of fluorochrome used, it's color, which clone and from which manufacturer.
Fitc: fluorescein isothiocyanate.
PE: R-Phycoerythrin.
ECD: Electron Coupled Dye.
PC5: Phycoerythrin-Cyanin 5.1,
PC7: Phycoerythrin-Cyanin 7

CFSE Based Cytotoxicity Assay

Flow cytometry-based cytotoxicity studies were performed to monitor the capability of UCB-EC cells to kill CML/AML target cells during co-incubation. Target cells were washed with PBS and labeled with 1 μM carboxyfluorescein diacetate succinimidyl ester (CFSE; Molecular Probes Europe, Leiden, The Netherlands) for 10 minutes at 37°. 5 ml IMDM with 10% FCS was added to terminate the reaction after which the cells were counted by fluorescence activated cell sorting (FACS). Cells were spun down resuspended in the same medium in the concencentration used. UCB-EC cells were counted as well by FACS and resuspended in the necessary concentration.

Target cells and UCB-EC cells were plated out alone in triplicates as controls. UCB-EC cell and AML/CML cells were co-cultured overnight at 37° C. in various E:T ratio's (1:1, 5:1) in a volume of 275 μl. Before overnight incubation α-CD107a-PE (BD Pharmingen, San Diego, Calif., USA) was added to check for degranulation. Before sample collection 70 μL of supernatant was taken and frozen for an ELISA assay. Cells were harvested and every sample was stained with 1 μl CD56-PC7 (N901, Beckman Coulter, Fullerton, USA) for at least 15 minutes to adjust the gate on UCB-EC cells and the number of lasting target cells was quantified using FACS based on CFSE positive staining (Error! Reference source not found.). The percentage toxicity was calculated by dividing the number of viable CFSE positive cells in coculture with UCB-EC cells by the number of viable CFSE positive target cells alone and multiplying this number by 100%.

$$Toxicity ( \% targets killed ) = 1 - ( \frac{Number of viable target cells in coculture}{Number of viable target cells alone} * 100 )$$

Enzyme-Linked Immuno Sorbent Assay (ELISA)

To quantify the interferon-γ (IFN-γ) production an ELISA was performed after CFSE based cytotoxicity assays. Maxisorp ELISA plates (Nunc) were coated overnight with 1.5 μg/ml 100 μl coating antibody anti-human IFN-γ (IgGq, 2G1, Endogen) in PBS at room temperature (RT). After incubation the antibody was removed and 200 μl blocking buffer (1% Bovine Serum Albumin (BSA) in PBS) was added for 1 hour at RT. Wells were washed 3 times with washing buffer (0.05% Tween (Merck) in PBS) and 50 μl of samples and human IFN-γ standard (Bender MedSystems) serial dilutions (2000 pg/ml to 0.85 pg/ml diluted in 1:1 IMDM+10% FCS & GBGM-CB+10% HS) were transferred to the coated plate. The plate was washed after another hour of sample incubation at RT followed by the addition of 50 μl 0.2 μg/ml biotin labeled monoclonal antibody (IgG1, 7-B6-1, Mabtech). Redudant antibody was washed away with washing buffer and 50 μl of a 1:12500 dilution of Horseradish Peroxidase (HRP) labeled streptavidine antibody (Sanquin) was incubated for another 30 minutes. After another washing step 100 μl of 1:1 mixture of TMB and Peroxidase B (TMB Microwell peroxidase Substrate System, KLP) was added to all coated wells. The plate was incubated until the two highest concentrations of IFN-γ standard had the same blue intensity (˜10 minutes) after which the enzymatic reaction was stopped with 100 μl 1 M H3HPO4 (Merck). Absorbance of this product was measured at 450 nm with a Multiscan MCC/340 ELISA reader (Titertek Instruments, Huntsville, USA).

The culture process is mainly divided into an expansion and a differentiation phase. For both phases a specific combination of various high- and low-dose cytokines and specific heparin are used to achieve cell expansion of highly pure and functional UCB-EC cell products. In order to optimize UCB-EC cell products and the production process for clinical or pharmaceutical purposes, we intent to assess the effect of each cytokine from the current cytokine combination as developed for UCB-EC cell differentiation. Therefore, UCB derived CD34+ stem cells were expanded for 2 weeks, according to the protocol as described previously⁷⁶. Pre-expanded UCB-EC progenitors were subsequently differentiated into mature and functional UCB-EC cells using 8 different high dose cytokine cocktails in the culture method(

). In all 8 conditions IL-15 was used, because IL-15 can induce expansion and differentiation of CD34+ hematopoietic progenitor cells into UCB-EC cells. With IL-15 as basis, all various combinations using IL-2, IL-7 and SCF were analyzed for their effect on expansion and differentiation of the UCB-EC cell product as well as their ability to lyse leukemic target cells.

First, we analyzed the expansion and differentiation rate as well as the purity of the UCB-EC cell culture. The mean total cell expansion was followed for 5 weeks and measured by flowcytometry. Moreover, cytokine expansion of UCB derived CD34+ cells was prominently affected by SCF addition to the high dose cytokine cocktail in all four donors. Results for three donors show a significant higher overall expansion of SCF cultures (20,222±11,423) versus non SCF cultures (6,546±2,690) (p<0.01). The addition of IL-2 or IL-7 had the second most positive effect. Secondly, the mean differentiation rate per cytokine combination was followed for 3 weeks and mainly all cytokine combinations results in the same high purity of the UCB-EC cell product.

CFSE-based cytotoxicity assays and IFN-γ ELISAs were used for the determination of UCB-EC cell functionality. Cytotoxicity assays were performed in a Effector:Target (ET) ratio of 1:1 to determine the effect of the high dose cytokine combination in the differentiation medium on UCB-EC cell mediated lysis. The results revealed, that ex vivo generated UCB-EC cells efficiently lyse HLA-devoid K562 target cells. UCB-EC cell mediated cytotoxicity for the UCB-EC cell product cultured with a high dose cytokine combination of IL-15 and IL-2 (64%±5%) is more cytotoxic, compared to IL-15 alone (42%±15%, p<0.05). The addition of SCF to IL-15 and IL-2 result in lower cytotoxicity of the UCB-EC cell product against K562 target cells (46%±8%, p<0.05). Additionally, we intended to study the produced interferon-gamma (IFN□γ□ of activated UCB-EC cells upon stimulation with different target cells. The assessment of IFN□γ□concentrations in the supernatant after a CFSE based cytotoxicity experiment could be used as an indication for UCB-EC cell activity during co-culture with leukemic targets. In summary, expansion was mostly improved by the addition of SCF to the high dose cytokine cocktail used for 3 weeks of differentiation culture. However, the purity of the resulting UCB-EC cell product does not increase by the addition of IL-2, IL-7 or SCF during the differentiation phase. Regarding functional analyzes, UCB-EC cell products cultured with IL-2 showed as increased lysis of K562, whereas SCF addition had a negative effect on the cytotoxicity. The results of all experiments were compared per high dose cytokine combination shows that best overall results were obtained when the 3 week differentiation culture of ex vivo UCB-EC cells was enriched with IL-15, SCF, IL-2 and IL-7.

TABLE 10
Matrix of properties of UCB-EC cell products high dose differentiate with
various combinations of high dose cytokines. Relative values were assigned
to the conditions specific based the experimental results. Ranking between
the different conditions within a property was performed according to the
experimental mean values. Absolute cell numbers seemed to be most
important for a UCB-EC cell product and therefore the values from
expansion were used to multiply the sum.

| | | | | | | | | IL-15 |
|---|---|---|---|---|---|---|---|---|
| | | | | | IL-15 | IL-15 | IL-15 | SCF |
| | | IL-15 | IL15 | IL-1 | IL-2 | SCF | SCF | IL-2 |
| | IL-15 | SCF | IL-2 | IL-7 | IL-7 | IL-2 | IL-7 | IL-7 |
| Purity | 1 | 1 | 1 | 1 | 1 | 1 | 1 | 1 |
| Functionality | | | | | | | | |
| Cyto vs.K562 | 1 | 1.5 | 3 | 2 | 2.5 | 1 | 1 | 2 |
| Cyto vs. KG1a | 1 | 1 | 1 | 1 | 1 | 1 | 1 | 1 |
| ELISA vs. K562 | 1 | 1.5 | 2 | 1 | 1.5 | 2 | 1.5 | 2 |
| ELISA vs. | 1 | 1 | 1 | 1 | 1 | 1 | 1 | 1 |
| KG1a | | | | | | | | |
| SUM | 5 | 6 | 8 | 6 | 7 | 6 | 5.5 | 7 |
| Expansion | 1 | 2 | 1 | 1 | 1 | 2.5 | 3 | 3 |
| Result | 5 | 12 | 8 | 6 | 7 | 15 | 16.5 | 21 |

Cyto = Cytotox data

Influence of IL-2 and IL-12 Cytokine Combinations, on Different Time Points During UCB-EC Cell Differentiation

In the initial experiments, the effect of each cytokine currently used in the cytokine cocktail developed for UCB-EC cell differentiation was studied. Whereas SCF has the highest influence on expansion and cell numbers, IL-2 affected the cytolytic function most positively. However, several other cytokines, like IL-12, IL-18 and IL-21, are known to exhibit significant effects on the functionality and activation of UCB-EC cells. One of those cytokines, IL-12, has been shown to induce proliferation, to stimulate production of cytokines such as IFN-g and lead to higher cytolytic function of UCB-EC cells. Moreover, IL-12 influences the surface receptor expression of UCB-EC cells.

In order to optimize UCB-EC cell products and the production process for clinical or pharmaceutical purposes, we intent to assess the effect of IL-2 and the additional IL-12 on various time points in the cytokine combination developed for UCB-EC cell differentiation. Therefore UCB derived CD34+ stem cells were expanded for 2 weeks, according to the protocol as described previously⁷⁶. Subsequently the ex vivo UCB-EC progenitors were differentiated ex vivo into UCB-EC cells using a high-dose cytokine combination of IL-15, SCF and IL-7 in all conditions. Additional cytokines IL-2 and/or IL-12 were added starting from week 2 onwards and at week 3 or 4 (scheme see FIG. 17). Those 12 different culture conditions were used to analyze the effect of IL-2 and/or IL-12 on different time points on the expansion, purity, cytotoxicity and maturation of the ex vivo generated UCB-EC cells.

The average results of all experiments were compared per high dose cytokine combination are displayed in table 11. Matrix of properties of UCB-EC cell products high dose differentiate with various combinations of IL-2 and IL-12. Relative values were assigned to the conditions based the experimental results. Ranking between the different conditions within a specific property was performed according to the experimental mean values. Absolute cell numbers seemed to be most important for a UCB-EC cell product and therefore the values from expansion were used to multiply the sum. Properties showing no differences were set 1. This overview shows that best overall results were obtained when the 5 week culture of ex vivo UCB-EC cells was enriched with IL-15, SCF, IL-7, and IL-12 from the start of differentiation phase with an addition of IL-2 2 weeks later.

TABLE 11
Matrix of properties of UCB-EC cell products vs. the high dose cytokine
combination used for 3 week culture. Relative values were assigned to the conditions
based the experimental results. Ranking between the different conditions within
a specific property was performed according to the experimental mean values.
Absolute cell numbers seemed to be most important for a UCB-EC cell product and
therefore the values from expansion were used to multiply the sum.

| | Condition | | | | | | | | | | | |
|---|---|---|---|---|---|---|---|---|---|---|---|---|
| | | | | | | — | | | | | | 2 |
| | — | — | — | — | — | 2 | 1 | 12 | 12 | 2 | 2 | 12 |
| | — | — | 12 | 12 | 2 | 12 | — | 2 | — | — | 12 | — |
| | — | 2 | — | 2 | — | — | — | — | 2 | — | — | — |
| Purity | 1 | 1 | 1 | 1 | 1 | 1 | 1 | 1 | 1 | 1 | 1 | 1 |
| Functionality | | | | | | | | | | | | |
| Cyto w4 | 1 | 1 | 2 | 2 | 2 | 2.5 | 3 | 3 | 3 | 2 | 2 | 2.5 |
| THP1 | | | | | | | | | | | | |
| Cyto w4 | 1 | 1 | 1 | 1 | 1 | 1 | 1 | 1 | 1 | 1 | 1 | 1 |
| KG1a | | | | | | | | | | | | |
| Cyto w4 | 1 | 1 | 1 | 1 | 1 | 1 | 1 | 1 | 1 | 1 | 1 | 1 |
| K562 | | | | | | | | | | | | |
| Cyto w5 | 1 | 4 | 5 | 5 | 1 | 5 | 5 | 6 | 6 | 3 | 4 | 6 |
| THP1 | | | | | | | | | | | | |
| Cyto w5 | | | | | | | | | | | | |
| KG1a | 1 | 1.5 | 2 | 2.5 | 1 | 2 | 2.5 | 2.5 | 3 | 1 | 2 | 3 |
| Cyto w5 | 1 | 1.5 | 2 | 2.5 | 1 | 2 | 2.5 | 2 | 3 | 1 | 1.5 | 1.5 |
| K562 | | | | | | | | | | | | |
| ELISA | 1 | 1 | 1 | 1 | 1.5 | 1.5 | 2 | 2 | 2 | 1.5 | 1 | 2 |
| week 4 | | | | | | | | | | | | |
| ELISA | 1 | 1 | 1 | 1.5 | 1.5 | 1.5 | 2 | 2 | 2 | 1 | 1.5 | 2 |
| week 5 | | | | | | | | | | | | |
| SUM | 9 | 13 | 16 | 16.5 | 11 | 17.5 | 20 | 20.5 | 22 | 12.5 | 15 | 20 |
| Expansion | 2 | 2.5 | 1.5 | 1.5 | 2.5 | 1.5 | 1 | 1 | 1.5 | 2 | 1 | 1 |
| Result | 1 | 32.5 | 24 | 24.7 | 27.5 | 26.2 | 20 | 20.5 | 33 | 25 | 15 | 20 |
| | 8 | | | 5 | | 5 | | | | | | |

Explanation for the condition:
Different weeks of culture are separated by a “dot” (.).
Cytokines IL-2 or IL-12 are indicated by 2 or 12.
( ) dash is indicating no addition of extra cytokine in this week.
Cyto = Cytotox data;
w = week;

TABLE 12
Cervical cancer cell line characteristics

| | | | | | | | | | | | Mean | |
|---|---|---|---|---|---|---|---|---|---|---|---|---|
| | | | | | | | | | | | MFI | |
| | | | Mean MFI-NK inhibitory ligands I | | | Mean MFI-NK cell activating ligands | | | | | EFFR | RAS |
| Cervical cancer cell lines | | | (n = 2) | | | (n = 2) | | | | | (n = 2) | typing |
| Cell line | Histology | HPV type | HLA-ABC | HLA-E | HLA-G | PVR | MICA/B | ULBP1 | ULBP3 | ULBP2/5/6 | EGFR | KRAS |
| HeLa | AC | 18 | 56.7 | 12.5 | 16.0 | 405.6 | 6.2 | 3.2 | 3.8 | 16.1 | 7.9 | Wild |
| | | | | | | | | | | | | type |
| TYPE | SCC | 16 | 55.8 | 20.4 | 29.9 | 422.6 | 8.5 | 7.0 | 4.2 | 114.8 | 26.5 | Wild |
| | | | | | | | | | | | | type |
| CaSki | Epider- | 16 | 35.6 | 17.9 | 19.4 | 392.8 | 10.7 | 6.7 | 10.6 | 55.2 | 93.0 | Wild |
| | moid | | | | | | | | | | | type |
| C33A | SCC | negative | 6.1 | 4.0 | 13.7 | 134.9 | 1.2 | 0.5 | 1.7 | 0.3 | 0.0 | Wild |
| | | | | | | | | | | | | type |
| CSCC7 | SCC | 16 | 36.8 | 12.7 | 14.7 | 186.5 | 0.6 | 2.5 | 2.7 | 57.0 | 41.4 | Wild |
| | | | | | | | | | | | | type |
| CC8 | ASC | 45 | 84.6 | 8.5 | 21.0 | 281.8 | 1.1 | 1.3 | 6.0 | 41.4 | 125.2 | Wild |
| | | | | | | | | | | | | type |
| CC10A | AC | 45 | 63.7 | 35.4 | 19.1 | 419.0 | 10.1 | 0.0 | 2.4 | 47.3 | 78.1 | Wild |
| | | | | | | | | | | | | type |
| CC10B | AC | 45 | 16.1 | 16.8 | 18.3 | 531.8 | 4.9 | 1.3 | 4.8 | 39.4 | 33.0 | Wild |
| | | | | | | | | | | | | type |
| CC11A | AC | 67 | 21.5 | 9.2 | 12.3 | 138.1 | 0.4 | 2.1 | 2.1 | 47.7 | 33.8 | Wild |
| | | | | | | | | | | | | type |
| CC11B | SCC | 67 | 14.3 | 10.7 | 10.6 | 152.4 | 1.4 | 2.0 | 4.7 | 12.2 | 27.7 | Wild |
| | | | | | | | | | | | | type |

### Example 8: Testing UCB-EC Ability to Overcome Tumor HLA-ABC, G and E Inhibition

Cervical cancer cell lines CSCC7, CC8, CC10A, CC10B, CC11A, and CC11B were generated in the department of Pathology of Leiden University Medical Center (The Netherlands) from primary tumors as described previously⁷⁹ These patient-derived cell lines as well as commercially obtained cervical cancer-derived cell lines, HeLa, SiHa, CaSki and C33A (ATCC) were maintained in Dulbecco's modified Eagle's (DMEM, Lonza) medium containing 4.5 g/L glucose, 10% FCS (Hyclone), 10 μg/mL gentamicin and 0.25 μg/ml amphotericin B (Gibco), 100 Units Penicillin/100 Units Streptomycin/0.3 mg/mL Glutamine (Thermo Fisher Scientific). Cell cultures were maintained at 37° C. in a humidified atmosphere containing 5% CO2. The targets cells (Hela, Siha, Caski, C33A, CSCC7, CC8, CC10A, CC10B, CC11A, and CC11B) were screened for HLA-ABC, HLA-G and HLA-E expression levels using flow cytometry.

### Phenotyping of Cervical Cancer Cell Lines

To phenotype cervical cancer cell lines, cell suspensions in PBS supplemented with 0.1% BSA and 0.02% NaN3 (FACS buffer) were stained for 30 min at 4° C. using antibodies to HLA-ABC (clone w6/32, Immunotools) (labeled with FITC), HLA-E (clone 3D12HLA-E, eBioscience), HLA-G (clone 87G, Biolegend). IgG1, IgG2a, and IgG2b isotype antibodies were used as negative controls. After incubation, the cells were washed with FACS buffer and analyzed using a flow cytometer LSR Fortessa (BD Biosciences). Screening for HLA-ABC, HLA-G and HLA-E expression were tested independently from different batch cultures of target cell lines over a period of 4 months. Phenotypic analyses were obtained from at least two independent experiments performed on each cell line. Data were analyzed using Kaluza software (Beckman coulter) and calculated as specific (geometric) mean fluorescence intensity (MFI) (MFI; geometric mean fluorescence of marker-geometric mean fluorescence of isotype). See Table 12 for NK inhibitory ligands expression levels. Further, Effector cells (UCB-EC and activated PBNK) were cultured with 10 cervical cancer cell lines expressing variable levels of HLA-ABC, HLA-G and HLA-E an inhibitory ligand for NK cell functions. 5×10⁴ effectors were co-cultured with 5×10⁴ targets (Hela, Siha, Caski, C33A, CSCC7, CC8, CC10A, CC10B, CC11A, and CC11B), E:T 1:1 for 4 hrs at 37° C. The percentage of target cell death induced by UCB-EC and PBNK are correlated with HLA-ABC, HLA-G and HLA-E levels of cervical cancer cell lines tested. From the results it was evident that UCB-EC can overcome tumor HLA-ABC inhibition significantly higher than activated PBNK cells (FIG. 18A, B), besides inducing effective tumor cell lysis of HLA-G (FIG. 19) and HLA-E (FIG. 20) expressing cell lines significantly higher than PBNK cells.

### Example 9: Influence of Human Papilloma Virus (HPV) Types and Tumor Histology on UCB-EC and PBNK Killing

To understand if UCB-EC, PBNK alone and PBNK+cetuximab tumor killing are influenced by different HPV types and/or tumor histology and to identify the most potent immune effector cell product among them, selected targets were grouped according to their i) different HPV types (C33A—HPV negative; HeLa—HPV 18; SiHa, CaSki, CSCC7—HPV 16; CC8, CC10A, CC10B—HPV 45; CC11A, CC11B—HPV 67) and ii) histology (HeLa, CC10A, CC10B, CC11A—Adenocarcinoma; SiHa, C33A, CSCC7, CC11B—Squamous cell carcinoma; CC8—Adenosquamous carcinoma; CaSki—Epidermoid). For PBNK+cetuximab conditions, target cells were coated with 5 μg/ml cetuximab, incubated at 4° C. for 1 hr. Cells were washed with PBS+0.05% BSA and added to effector cells for cytotoxicity assays.

### Effector Cell Preparation

Peripheral venous blood samples were collected in tubes containing sodium heparin anticoagulant. Peripheral blood mononuclear cells (PBMCs) were isolated by density-gradient centrifugation with using Lymphoprep™ (STEMCELL Technologies, The Netherlands) washed and resuspended in MACS buffer (PBS+0.05% BSA) for isolation of peripheral blood NK cells using Human NK cell isolation kit (Miltenyi Biotech, Bergisch Gladbach, Germany) according to the manufacturer's instructions. Isolated NK cells are activated overnight with 1000U/ml IL-2 (Proleukin®; Chiron, München, Germany) and 10 ng/ml IL-15 (CellGenix) for use in cytotoxicity assays. NK cell purity and viability are checked using CD3 PE, 7AAD (BD Biosciences), CD56 APC Vio 770, and CD16 APC (Miltenyi Biotech).

### Target Cell Preparation:

Cell lines, Hela, Siha, Caski, C33A, CSCC7, CC8, CC10A, CC10B, CC11A, CC11B (cervical carcinoma) are obtained from ATCC or cell stock from patient derived cell lines (Leiden university) and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator. Target cells were stained with 5 μM pacific blue succinimidyl ester (PBSE; Molecular Probes Europe, Leiden, The Netherlands) in a concentration of 1×10⁷ cells per ml for 10 min at 37° C. The reaction is terminated by adding an equal volume of FCS, followed by incubation at room temperature for 2 min after which stained cells are washed twice with 5 ml DMEM/10% FCS. After washing, cells are suspended in DMEM/10% FCS to a final concentration of 5×10⁵/ml.

### Flow Cytometry Based NK Cell Cytotoxicity Assay

PBSE stained targets untreated and treated with cetuximab were co-cultured with different HPV positive and negative targets and their cytotoxicity was compared to UCB-EC cells. PBSE positive and CD45⁺CD56⁺ staining were used to discriminate target and effector cells. 7AAD was used to detect target cell death and the percentage of dead target cells was calculated from FACS plots showing 7AAD uptake on PBSE+ targets. NK cells alone, NK cells treated with cetuximab, Target cells alone and cetuximab treated target cells alone were used as control samples. Target and effector cells were incubated for 4h with an effector:target ratio of 1:1. The FIGS. 21 and 22 are representative of five identical experiments. From the results obtained it was evident that both PBNK and UCB-EC killing was not influenced by HPV types and tumor histology and more interestingly UCB-EC cells killed all tumor types independent of HPV (FIG. 21) and histology (FIG. 22) at significantly higher levels than PBNK and are equally cytotoxic as PBNK+cetuximab conditions.

### Example 10

Antitumor efficacy against EGFR^{(negative, low and high)} expressing cervical cancer cells by UCB-EC compared with PBNK cells and PBNK cells coated with cetuximab using flow cytometry based cytotoxicity and degranulation assays according to the basic protocols as described before⁷⁶.

### Cell Lines

Cell lines, Hela, Siha, Caski, C33A, CSCC7, CC8, CC10A, CC10B, CC11A, CC11B (cervical carcinoma) are obtained from ATCC or cell stock from patient derived cell lines (Leiden university) and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator.

### Phenotyping of Cervical Cancer Cell Lines

To phenotype cervical cancer cell lines for EGFR, cell suspensions in PBS supplemented with 0.1% BSA and 0.02% NaN3 (FACS buffer) were stained for 30 min at 4° C. using antibodies, EGFR (clone EGFR.1, BD Biosciences) labeled with phycoerythrin (PE)). IgG2b isotype antibodies were used as negative controls. After incubation, the cells were washed with FACS buffer and analyzed using a flow cytometer LSR Fortessa (BD Biosciences). Screening for target cells EGFR expression were tested independently from different cultures of target cell lines over a period of 4 months. Phenotypic analyses were obtained from at least two independent experiments performed on each cell line. Data were analyzed using Kaluza software (Beckman coulter) and calculated as specific (geometric) mean fluorescence intensity (MFI) (MFI; geometric mean fluorescence of marker—geometric mean fluorescence of isotype). See Table 12 for cervical cancer cell line EGFR expression levels.

Isolation and Activation of Peripheral Blood NK Cells from Whole Blood Specimens

Whole blood from healthy volunteers is collected with written informed consent. Mononuclear cells (MNCs) are isolated using Lymphoprep™ (STEMCELL Technologies, The Netherlands) density gradient centrifugation. PBNK cells are isolated from MNCs using a MACS Human NK cell isolation kit (Miltenyi Biotech, Bergisch Gladbach, Germany) according to the manufacturer's instructions. The cell number and purity of the isolated NK cell fraction are analyzed by flow cytometry. Isolated NK cells are activated overnight with 1000U/ml IL-2 (Proleukin®; Chiron, München, Germany) and 10 ng/ml IL-15 (CellGenix) for use in cytotoxicity assays. NK cell purity and viability are checked using CD3 PE, 7AAD (BD Biosciences), CD56 APC Vio 770, and CD16 APC (Miltenyi Biotech). The preliminary parameters noted before and after activation are NK purity (CD56+%, 83 ±9% & 82 ±9%), NK CD16% 88 ±10% & 85 ±11%) and NK viability (91 ±3% & 86 ±2%) respectively.

### UCB-EC Cell Cultures

### Ex Vivo Expansion of CD34-Positive Progenitor Cells

CD34+ UCB cells (between 1×10⁴ and 3×10⁵ per ml) are plated into 24-well tissue culture plates (Corning Incorporated, Corning, N.Y.) in GBGM supplemented with 10% human serum (HS; Sanquin Bloodbank, Nijmegen, The Netherlands), 20 ng/mL of SCF, Flt-3L, TPO, IL-7 (all CellGenix). From Day 9-14, TPO is replaced with 20 ng/mL IL-15 (CellGenix) in the expansion cultures. During the first 14 days of culture, low molecular weight heparin (LMWH) (Clivarin®; Abbott, Wiesbaden, Germany) is added to the expansion medium in a final concentration of 25 μg/ml and a low-dose cytokine cocktail consisting of 10 pg/ml GM-CSF, 250 pg/ml G-CSF, (Stemcell Technologies) and 50 pg/ml IL-6 (CellGenix, Freiburg, Germany). Cell cultures are refreshed with new medium every 2-3 days. Cultures are maintained in a 37° C., 95% humidity, 5% CO2 incubator.

Differentiation of Ex Vivo Expanded CD34⁻Positive Cells into UCB-EC Cells

Expanded CD34+ UCB cells are differentiated and further expanded using effector cell differentiation medium. This medium consists of the same basal medium as used for the CD34 expansion step supplemented with 2% HS, the low-dose cytokine cocktail (as previously mentioned) and a new high-dose cytokine cocktail consisting of 20 ng/ml of IL-7, SCF, IL-15 (CellGenix) and 1000 U/ml IL-2 (Proleukin®; Chiron, München, Germany) is added to the differentiation medium. Medium is refreshed twice a week from day 14 onwards.

### Flow Cytometry

Flow cytometry analysis is performed on a BD LSR FORTESSA X-20 (BD Biosciences). Cell numbers and expression of cell-surface markers are determined by flow cytometry. The cell numbers and the population of live cells is determined by gating on CD45⁺ cells based on forward scatter (FSC) and side scatter (SSC). For analysis of phenotype, the cells were gated only on FSC/SSC and further analyzed for the specific antigen of interest. Cells were incubated with the appropriate concentration of antibodies for 30 min at 4° C. After washing, cells are suspended in FACS buffer.

### Flow Cytometry-Based Cytotoxicity and Degranulation Studies

Flow cytometry is used for the read-out of cytotoxicity assays. Target cells are labeled with 5 μM pacific blue succimidyl ester (PBSE; Molecular Probes Europe, Leiden, The Netherlands) in a concentration of 1×10⁷ cells per ml for 10 min at 37° C. The reaction is terminated by adding an equal volume of FCS, followed by incubation at room temperature for 2 min after which stained cells are washed twice with 5 ml DMEM/10% FCS. After washing, cells are suspended in DMEM/10% FCS to a final concentration of 5×10⁵/ml. PBNK and UCB-EC cells are washed with PBS and suspended in Glycostem Basal Growth Medium (GBGM)+2% FCS to a final concentration of 5×10⁵/ml. Target cells are co-cultured with effector cells at an E:T ratio of 1:1 in a total volume of 250 μl in 96-wells flat-bottom plates (5×10⁴ targets in 100 μl of DMEM+10% FCS incubated with 5×10⁴ effectors in 100 μl of GBGM+2% FCS, further supplemented with 25 μl of GBGM+2% FCS and DMEM+10% FCS medium). PBNK cells, UCB-EC cells and target cells alone are plated out in triplicate as controls. To measure degranulation by PBNK and UCB-EC cells, anti-CD107a PE (Miltenyi Biotech, Germany) is added in 1:20 dilution to the wells. After incubation for 4 h at 37° C., 75 μl supernatant is collected and stored at −20° C. for analysis of cytokine production. Cells in the remaining volume are harvested and stained with 7AAD (1:20). Degranulation of PBNK and UCB-EC cells is measured by detecting cell surface expression of CD107a. After 4 hrs of incubation at 37° C., CD56 APC Vio 770 (1:25) and CD16 APC (1:25) (Miltenyi Biotech, Germany) are added to the co-cultures and NK CD107a degranulation is measured for CD56+ PBNK and UCB-EC cells.

### Statistical Analysis

Statistical analysis is performed using Graph Pad Prism software. Differences between conditions are determined using one way Anova, two way Anova with multiple comparisons between column means and student's T test. Results from cytotoxicity experiments are described as mean±standard deviation of the mean (SD). A p-value of <0.05 is considered statistically significant.

Data from clinical studies in cervical cancer patients, clearly points out that anti-EGFR mAb therapy (cetuximab) was ineffective in EGFR expressing RAS wild type patients⁸⁰. To confirm their findings, we studied cervical cancer cell lines Hela, Siha, Caski, CSCC7, CC8, CC10A, CC10B, CC11A, and CC11B, except C33A which expresses EGFR for anti-tumor effects of cetuximab monotherapy in vitro. In line with previous studies, cetuximab as monotherapy did not induce cell death in any of the cell lines tested (FIG. 23).

Next, activated PBNK were compared with UCB-EC for their ability to induce target cell death. UCB-EC were significantly more cytotoxic than PBNK, consistently inducing higher rates of tumor cell death in all tested cell lines (P<0.001) (FIG. 24A, B). This was further borne out by observed degranulation levels of NK cells in response to exposure to the cervical cancer cell lines, as measured by CD107a surface expression. These were comparably and significantly elevated in the PBNK+cetuximab and UCB-EC conditions over PBNK alone (FIG. 24C). Interestingly, PBNK degranulation levels were low in combination with cetuximab upon exposure to cervical cancer cell lines expressing low levels of EGFR (C33a, HeLa and SiHa: denoted in FIG. 19C by triangles). In contrast, degranulation levels in UCB-EC were invariably high (FIG. 24C).

### Example 11

UCB-EC Share a Common Functional Homology with PBNK Cells

### Cell Lines

Cell lines, C33A and SiHa (cervical carcinoma) are obtained from ATCC and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator.

### Phenotyping of Cervical Cancer Cell Lines

To phenotype C33A and SiHa, cell suspensions in PBS supplemented with 0.1% BSA and 0.02% NaN3 (FACS buffer) were stained for 30 min at 4° C. using antibodies to PVR (clone SK11.4, Biolegend), MICA/B (clone 6D4, Biolegend), ULBP2/5/6 (clone #165903, R&D systems), ULBP1 (clone #170818, R&D systems) and ULBP3 (clone #166510, R&D systems), (all labeled with phycoerythrin (PE)). IgG1, IgG2a, and IgG2b isotype antibodies were used as negative controls. After incubation, the cells were washed with FACS buffer and analyzed using a flow cytometer LSR Fortessa (BD Biosciences). Screening for PVR (ligand for DNAM-1) and MICA/B, ULBP1, ULBP2/5/6, ULBP3 (ligands for NKG2D) were tested independently from different batch cultures of target cell lines over a period of 4 months. Phenotypic analyses were obtained from at least two independent experiments performed on each cell line. Data were analyzed using Kaluza software (Beckman coulter) and calculated as specific (geometric) mean fluorescence intensity (MFI) (MFI; geometric mean fluorescence of marker-geometric mean fluorescence of isotype). See Table 12 for NK activating ligands expression levels.

### UCB-EC Cultures for Blocking Studies

UCB-EC cells were generated from cryopreserved UCB hematopoietic stem cells as previously described^{72,76}. CD34⁺ UCB cells (3×10⁵ per ml) were plated into 12-well tissue culture plates (Corning Incorporated, Corning, N.Y.) in Glycostem Basal Growth Medium (GBGM®) (Clear Cell Technologies, Beernem, Belgium) supplemented with 2% human serum (HS; Sanquin Bloodbank, The Netherlands), 20 pg/mL of SCF, Flt-3L, TPO, IL-7 (CellGenix). In the expansion phase II, from day 9 to 14, TPO was replaced with 20 pg/mL IL-15 (CellGenix). During the first 14 days of culture, low molecular weight heparin (LMWH) (Clivarin®; Abbott, Wiesbaden, Germany) in a final concentration of 25 μg/ml and a low-dose cytokine cocktail consisting of 10 pg/ml GM-CSF, 250 pg/ml G-CSF, (Stemcell Technologies) and 50 pg/ml IL-6 (CellGenix, Freiburg, Germany) were added to the expansion cultures. Cells were refreshed with new medium twice a week and maintained at 37° C., 5% CO₂. On day 14, NK cell differentiation process was initiated by addition of NK cell differentiation medium. It consists of the same basal medium with 2% HS and low dose cytokine cocktail as the expansion steps with a new high-dose cytokine cocktail consisting of 20 ng/ml of IL-7, SCF, IL-15 (CellGenix) and 1000 U/ml IL-2 (Proleukin®; Chiron, München, Germany). Cultures were refreshed every 2-3 days and maintained till day 35. For cytotoxicity assays, UCB-EC were used with CD56⁺ cells >85% purity.

### UCB-EC and PBNK Blocking Cytotoxicity Assays

Cervical cancer cell lines (C33A and SiHa) were labeled with pacific blue succimidyl ester (PBSE; Molecular Probes Europe, Leiden, The Netherlands) in a concentration of 1×10⁷ cells per ml for 15 min at 37° C. After incubation, cells were resuspended in DMEM culture medium containing 10% FCS, gentamicin/amphotericin B, and Penicillin/Streptomycin/Glutamine, to a final concentration of 5×10⁵/ml. PBNK and UCB-EC were washed with PBS and suspended in GBGM medium with 2% FCS to a final concentration of 5×10⁵/ml. Target cells were co-cultured with effector cells (PBNK or UCB-EC), with or without the presence of 5 μg/ml cetuximab at an E:T ratio of 1:1 in a total volume of 100 μl in FACS tubes (5×10⁴ targets in 50 μl of culture medium incubated with 5×10⁴ effectors in 50 μl of GBGM medium). PBNK, UCB-EC and target cells alone were cultured in triplicate as controls. To measure degranulation by PBNK and UCB-EC, anti-CD107a PE (Miltenyi Biotech, Germany) was added at the beginning of the assay. After incubation for 4h at 37° C., cells were harvested and stained with 7AAD, CD56 (labeled with APC-Vio770) and CD16 (labeled with APC) (all from Miltenyi Biotech, Germany) were added to the co-cultures and NK CD107a degranulation was measured for PBNK and UCB-EC.). For UCB-EC and PBNK blocking experiments NKG2D PE (clone ON72, Beckman Coulter) and DNAM-1 (clone DX11, BD Pharmingen™) were used at 10 μg/ml. UCB-EC and PBNK cells were incubated with DNAM-1 and NKG2D blocking antibodies for 1 hr at 4° C. BD LSR Fortessa™ was used for read-out of the cytotoxicity assays. NK activating receptors blocking studies were also performed in the similar set up of cytotoxicity assays as described above. Flow cytometer was used for the read-out of cytotoxicity assays.

To investigate the role of activating receptors in the cytotoxicity of PBNK and UCB-EC, two major NK activating receptors NKG2D and DNAM-1 and their ligands MICA/B, ULBPs (NKG2D), PVR (DNAM-1) were studied. From the panel of cell lines screened for NK activating ligands as shown in FIG. 27A, SiHa (with highest expression levels of PVR and ULBP-2/5/6) and C33A (with lowest expression levels of PVR and ULBP-2/5/6), were chosen as target cells and blocking experiments were performed in a similar set up of NK cytotoxicity assays as described above. See table 12 for MFI levels of NK activating ligands. In case of C33A, only combined blocking of DNAM-1 and NKG2D led to a significant reduction in their susceptibility to PBNK and UCB-EC killing than individual blocking. The low levels of NK activating ligands expressed on C33A cells required a combined action to have significant impact on C33A cells. However in SiHa, a much stronger effect of blocking was observed in DNAM-1 and NKG2D only conditions, with no differences seen on combination, well explained by their high expression of NKG2D and DNAM-1 ligands required for NK recognition (FIG. 27B). Both the effectors had a similar response to blocking NKG2D and DNAM-1 and this experiment stresses the need for sufficient levels of NKG2D and DNAM-1 receptors on NK cells and ligands present on target cells to enhance NK cytotoxicity.

### Example 12: UCB-EC Exhibits Higher Cytotoxic Efficacy Against IDO Overexpressing Cells Compared to PBNK Cells

### Cell Lines

Cell lines, CaSki and SiHa (cervical carcinoma) are obtained from ATCC and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures are passaged every 5 days and maintained in a 37° C., 95% humidity, 5% CO2 incubator.

### PBMC Isolation & NK Cell Isolation

Whole blood samples from four healthy volunteers were collected. Peripheral blood mononuclear cells (PBMCs) were isolated using Lymphoprep™ (STEMCELL Technologies, The Netherlands) density gradient centrifugation. CD56+ NK cells were isolated from PBMCs using a MACS® Human NK cell isolation kit (Miltenyi Biotech, Bergisch Gladbach, Germany) according to the manufacturer's instructions. The cell number and purity of the isolated PBNK was analyzed by flow cytometry. Isolated NK cells were activated overnight with 1000 U/ml IL-2 (Proleukin®; Chiron, München, Germany) and 10 ng/ml IL-15 (CellGenix) before use in cytotoxicity assays. NK cell purity and viability were checked by flow cytometry using the following antibodies: 7-Aminoactinomycin D (7AAD; Sigma Aldrich), CD3 (labelled with VioBlue), CD56 (labelled with APC-Vio770), and CD16 (labelled with APC) (all from Miltenyi Biotech). For cytotoxicity assays, only PBNK cells with CD16 expression rates exceeding 80% were used.

### UCB-EC Isolation and Cultures

Allogeneic NK cells (UCB-EC) were generated from cryopreserved umbilical cord blood (UCB) hematopoietic stem cells as previously described (32). CD34+UCB cells (3×10⁵ per ml) were plated into 12-well tissue culture plates (Corning Incorporated, Corning, N.Y.) in Glycostem Basal Growth Medium (GBGM®) (Clear Cell Technologies, Beernem, Belgium) supplemented with 2% human serum (HS; Sanquin Bloodbank, The Netherlands), 20 μg/mL of SCF, Flt-3L, TPO, and IL-7 (CellGenix). In the expansion phase II, from day 9 to 14, TPO was replaced with 20 μg/mL IL-15 (CellGenix). During the first 14 days of culture, low molecular weight heparin (LMWH) (Clivarin®; Abbott, Wiesbaden, Germany) in a final concentration of 25 μg/ml and a low-dose cytokine cocktail consisting of 10 pg/ml GM-CSF (Neupogen), 250 pg/ml G-CSF and 50 pg/ml IL-6 (CellGenix, Freiburg, Germany) were added to the expansion cultures. Cells were refreshed with new medium twice a week and maintained at 37° C., 5% CO2. On day 14, the NK cell differentiation process was initiated by addition of NK cell differentiation medium consisting of the same basal medium with 2% HS but with high-dose cytokine cocktail consisting of 20 ng/ml of IL-7, SCF, IL-15 (CellGenix) and 1000 U/ml IL-2 (Proleukin®; Chiron, München, Germany). Cultures were refreshed every 2-3 days and maintained till day 35. For cytotoxicity assays, UCB-EC were used with CD56+ cells >85% purity.

### Flow Cytometry-Based Cytotoxicity and Degranulation Studies

Flow cytometry was used for the read-out of cytotoxicity assays. Target cells were labeled with 5 μM pacific blue succimidyl ester (PBSE; Molecular Probes Europe, Leiden, The Netherlands) in a concentration of 1×10⁷ cells per ml for 10 min at 37° C. The reaction was terminated by adding an equal volume of FCS, followed by incubation at room temperature for 2 min after which stained cells were washed twice with 5 ml DMEM/10% FCS. After washing, cells were suspended in DMEM/10% FCS to a final concentration of 5×10⁵/ml. CD56+ NK cells were washed with PBS and suspended in Glycostem Basal Growth Medium (GBGM)+2% FCS to a final concentration of 5×10⁵/ml. Target cells were co-cultured with effector cells at an E:T ratio of 1:1 in a total volume of 250 μl in 96-wells flat-bottom plates (5×10⁴ targets in 100 μl of DMEM+10% FCS incubated with 5×10⁴ effectors in 100 μl of GBGM+2% FCS, further supplemented with 25 μl of GBGM+2% FCS and DMEM+10% FCS medium). NK cells and target cells alone were plated out in triplicate as controls. Target cells (CaSki and SiHa) were coated with NKG2D and DNAM-1 blocking antibodies for 1h at 4° C. Cells were washed and co cultured with activated PBNK and UCB-EC cells. To measure degranulation by NK cells, anti-CD107a PE (Miltenyi Biotech, Germany) was added in 1:20 dilution to the wells. After incubation for 4h at 37° C., Cells were harvested and stained with 7AAD (1:20). Degranulation of NK cells was measured by detecting cell surface expression of CD107a. After 4 hrs of incubation at 37° C., CD56 APC Vio 770 (1:25) and CD16 APC (1:25) (Miltenyi Biotech, Germany) were added to the co-cultures and NK CD107a degranulation was measured for CD56+NK, CD56+CD16+NK and CD56+CD16− NK cells.

In cervical cancer patients, increased levels of immunosuppressive enzyme indoleamine-2, 3-dioxygenase (IDO) are found, which might be able to block immune effector functions and facilitates tumor growth^{39,81-83}. It has been shown that downregulation of IDO can result in increased NK cell accumulation in the tumor stroma in vivo, besides enhanced sensitivity to PBNK killing⁸⁴. Clinical studies also point out that there is significant downregulation of NK natural cytotoxicity receptors (NKp30 and NKp46) and NKG2D in cervical cancer patients directly affecting the functions of patient's PBNK⁸⁵. Comparing target cell death induced by PBNK and UCB-EC for IDO expressing cell lines both SiHa and CaSki were killed at significantly higher levels by UCB-EC (FIG. 28). This provides an ideal platform to target IDO expressing cervical cancer cells with UCB-EC and possibly in combination with IDO blockers to mount a stronger effect on cervical cancer cells. The ability of UCB-EC to overcoming the resistance of IDO and provides an excellent opportunity to treat cervical cancer tumors with UCB-EC.

### Example 13

Ex vivo-generated allogeneic immune effector cells are infused into poor-prognosis acute myeloid leukemia (AML) patients following cyclophosphamide/fludarabine (Cy/Flu) conditioning. This immunosuppressive conditioning regimen is necessary to prevent rejection and has shown to induce immune effector cell survival factors such as IL-15 that facilitate prolonged in vivo lifespan and expansion of the infused immune effector cells. The immune effector cell products are >70% for Neural Cell Adhesion Molecule (NCAM) expression and almost devoid of CD3+ T cells, thereby minimizing donor T cell-mediated GVHD. Study participants will undergo clinical and immunological evaluation. After achieving complete remission (<5% blasts in bone marrow) following one or two induction chemotherapy courses patients are typed for HLA class I alleles by serological testing and polymerase chain reaction (PCR-SSOP) and tested for the absence of anti-HLA antibodies using a standard Luminex protocol. Eligible AML patients are those without anti-HLA antibodies and for whom a allogeneic non-haploidentical UCB unit displaying an available HLA match for HLA-A and HLA-B at antigen level can be found in a pool of 50 randomly selected UCB units. HLA-DRB1, HLA-DQ and HLA-DP matching have not been used for UCB unit selection. Immediately after allocation, while consolidation chemotherapy is performed according to standard protocol, available UCB units are screened for selecting an appropriate donor for ex vivo immune effector cell expansion.

Six weeks prior to immune effector infusion, the suitable allogeneic UCB unit is thawed and CD34+ cells are enriched by using a CliniMACS cell separator after binding with CD34 coupled to immunomagnetic particles (Miltenyi Biotec). Enriched CD34+ UCB cells are used for ex vivo generation of NCAM positive immune effector cell products, through differentiation and expansion, according to the validated procedure⁷². Cell isolation, enrichment and culture procedures are performed under Good Manufacturing Practice (GMP) conditions in a clean room, using established SOPs according to JACIE, NETCORD FACT guidelines and EU directive 2001/83 and 2009/120.

### Example 14: Enhanced Cytotoxicity by UCB-EC Cells Against Colon Cancer Cells In Vitro

### Cell Lines

Cell lines A431 (epidermoid carcinoma), COLO320, SW480 and HT-29 (colon carcinoma) were obtained from American Type culture collection (ATCC) and cultured in Dulbecco's modified medium (DMEM; Invitrogen, Carlsbad Calif., USA) containing 100 U/ml penicillin, 100 μg/ml streptomycin and 10% fetal calf serum (FCS; Integro, Zaandam, The Netherlands). Cell cultures were passaged every 5 days and maintained in a 37° C., 95% humidity, 5% CO2 incubator.

### PBMC and PBNK Isolation

Peripheral blood mononuclear cells (PBMC) were isolated from the heparinized blood of healthy donors and colorectal cancer patients with informed consent. PBMC were isolated using Lymphoprep™ (STEMCELL Technologies, Cologne, Germany) density gradient centrifugation. CD56⁺ NK cells were isolated from PBMC using a MACS Human NK cell isolation kit (Miltenyi Biotech, Bergisch Gladbach, Germany) according to the manufacturer's instructions. PBNK cell purity and viability were checked using CD3 VioBlue, CD56 APC Vio 770, and CD16 APC (Miltenyi Biotech) and 7AAD (BD Biosciences). The parameters compared before and after stimulation with cytokines were NK purity (CD56+%, 87±5% vs. 84±2%), NK CD16% 92±12% vs 88±8%) and NK viability (89±5% vs 84±8%) respectively. Isolated PBNK cells were activated overnight with 1000U/ml IL-2 (Proleukin®; Chiron, München, Germany) and 10 ng/ml IL-15 (CellGenix) for use in cytotoxicity assays.

### UCB-EC Cultures

Allogeneic NK cells (UCB-EC) were generated from cryopreserved umbilical cord blood (UCB) hematopoietic stem cells as previously described⁷⁶. CD34⁺ UCB cells from six UCB-donors were plated (4×10⁵/ml) into 12-well tissue culture plates (Corning Incorporated, Corning, N.Y., USA) in Glycostem Basal Growth Medium (GBGM®) (Clear Cell Technologies, Beernem, Belgium) supplemented with 2% human serum (HS; Sanquin Bloodbank, Amsterdam, The Netherlands), 20 μg/mL of SCF, Flt-3L, TPO, and IL-7 (CellGenix Freiburg, Germany). In the expansion phase II, from day 9 to 14, TPO was replaced with 20 μg/mL IL-15 (CellGenix). During the first 14 days of culture, low molecular weight heparin (LMWH) (Clivarin®; Abbott, Wiesbaden, Germany) in a final concentration of 25 μg/ml and a low-dose cytokine cocktail consisting of 10 pg/ml GM-CSF (Neupogen), 250 pg/ml G-CSF and 50 pg/ml IL-6 (CellGenix) were added to the expansion cultures. Cells were refreshed with new medium twice a week and maintained at 37° C., 5% CO2. On day 14, the NK cell differentiation process was initiated by addition of NK cell differentiation medium consisting of the same basal medium with 2% HS but with high-dose cytokine cocktail consisting of 20 ng/ml of IL-7, SCF, IL-15 (CellGenix) and 1000 U/ml IL-2 (Proleukin®; Chiron, München, Germany). Cultures were refreshed every 2-3 days and maintained till day 42. For cytotoxicity assays, five UCB-EC cultures were used with CD56⁺ cells >92% purity and one UCB-EC unit was expanded on a large scale for mice studies and used with a CD56⁺ cells purity of 95%.

### NK Cell Cytotoxicity Assays

Flow cytometry was used for the read-out of cytotoxicity assays. Target cells (COLO320, SW480 and HT-29 were labelled with 5 μM pacific blue succinimidyl ester (PBSE; Molecular Probes Europe, Leiden, The Netherlands) in a concentration of 1×10⁷ cells per ml for 10 min at 37° C. The reaction was terminated by adding an equal volume of FCS, followed by incubation at room temperature for 5 min after which stained cells were washed twice and suspended in DMEM+10% FCS to a final concentration of 5×10⁵/ml. Overnight activated PBNK cells and UCB-EC cells were washed with PBS and suspended in Glycostem Basal Growth Medium (GBGM)+2% FCS to a final concentration of 5×10⁵/ml. Target cells were co-cultured with effector cells at an E:T ratio of 1:1 in a total volume of 250 μl in 96-wells flat-bottom plates (5×10⁴ targets in 100 μl of DMEM+10% FCS incubated with 5×10⁴ effectors in 100 μl of GBGM+2% FCS, further supplemented with 25 μl of GBGM+2% FCS and DMEM+10% FCS medium). NK cells and target cells alone were plated out in triplicate as controls. Target cells were coated with for 1h at 4° C. To measure degranulation by NK cells, anti-CD107a PE (Miltenyi Biotech, Germany) was added in 1:20 dilution to the wells. After incubation for 4h at 37° C. Cells in the remaining volume were harvested and stained with 7AAD (1:20). Degranulation of NK cells was measured by detecting cell surface expression of CD107a. After 4 hrs of incubation at 37° C., CD56 APC Vio 770 (1:25) and CD16 APC (1:25) (Miltenyi Biotech, Germany) were added to the co-cultures and NK CD107a degranulation was measured for CD56+ NK, CD56+CD16+ NK and CD56+CD16− NK cells.

### Anti-EGFR Monoclonal Antibody

Cetuximab (Merck, Darmstadt, Germany) was purchased from VU medical center pharmacy for NK cell ADCC experiments.

In advanced CRC, there is an immediate need to develop and explore novel therapies to replace dysfunctional NK cells, which can also probably target drug resistant tumors. In this study we tested two different sources of allogeneic NK cell products to find an NK alternative, further enhancing CRC patient's immune system. To characterize their functional role, a series of in vitro NK cytotoxicity assays were set up between A-PBNK cells and UCB-EC cells. Three different cell lines of colon cancer origin were used; from the results it was evident that both NK cells were capable of inducing cytolysis independent of EGFR and RAS status. In case of COLO320, which is EGFR negative, the added effect of cetuximab was not seen, but were killed by UCB-EC cells alone at a significantly higher level (p<0.01) than A-PBNK cells. For EGFR+RASmut SW480 and EGFR⁺ BRAF^{mut} HT-29, combination of A-PBNK+CET enhanced tumor killing via ADCC, and their killing levels were comparable to UCB-EC cells (FIG. 31A). UCB-EC cells were unable to perform ADCC in combination with cetuximab due to low CD16 levels in vitro⁷³. Similarly, NK degranulation was reflective of NK killing for the cell lines tested (FIG. 31B).

These results show that UCB-EC cells have superior cytotoxic efficacy than A-PBNK cells against cetuximab resistant colon cancer cells in vitro.

### Example 15: UCB-EC Inhibits Tumor Growth and Metastasis In Vivo

### Target Cells Lentiviral Infection

EGFR⁺ RAS^{wt} A431 and EGFR⁺ RAS^{mut} SW480 cell lines were stably transduced with Gaussia Luciferase (Gluc) for in vivo studies. Lentiviral (LV) supernatants of Cerulean Fluorescent Protein (CFP) positive Gluc virus (LV-CFP-Gluc) was kindly provided by Thomas Wudringer, manufactured according to a protocol described in Wudringer et al., Nat Protoc. 2009; 4(4): 582-591)⁸⁶. Cells were sorted twice to achieve higher purity and transduction efficacy was checked using flow cytometry. SW480 cells with Gluc purity <95% were used for tumor injection in mice.

### Mice

Immunodeficient BRGS mice (BALB/c Rag2^{tm1Fwa} IL-2R_{γc}^{tm1Cgn} SIRPα^{NOD}) were used in this study¹. 24 adult mice were injected intravenously (i.v) via tail vein with 0.5×10⁶ SW480 Gluc cells and were randomized into 4 groups, SW480 only (I), SW480+cetuximab (II), SW480+UCB-EC (III) and SW480+UCB-EC+cetuximab (IV). 30×10⁶ UCB-EC were infused i.v per mice on days 1, 3 and 7 post tumor injection, 10×10⁶ cells per injection) for treatment groups III & IV. Similarly, 0.5 mg per mice cetuximab was injected intra peritoneal (i.p) for groups II & IV on days 1, 3 and 7. Treatment effects were monitored using blood Gluc levels and bioluminescence imaging (BLI). All manipulations of BRGS mice were performed under laminar flow conditions.

### Ethics Statement

Animals were housed in isolators under pathogen-free conditions with humane care and anesthesia was performed using inhalational isoflurane anaesthesia to minimize suffering. Experiments were approved by an ethical committee at the Institute Pasteur (Reference #2007-006) and validated by the French Ministry of Education and Research (Reference #02162.01).

### Blood Gluc Quantification In Vitro

Secreted Gluc was measured according to a protocol previously described2. 10 μl of blood were collected by capillarity into EDTA containing Microvette® CB tubes. Blood samples were distributed in 96 well black plates and then mixed with 100 μl of 100 mM Gluc substrate native coelenterazine in PBS (P.J.K. GmbH; Kleinblittersdorf, Germany). Blood withdrawn before tumor inoculation served as a baseline value. Measurements were done twice a week till day 35. Gluc activity was measured using luminometer using the IVIS spectrum in vivo imaging system (PerkinElmer).

### Bioluminescence Imaging

Mice were anesthetized with using isofluorane gas in an induction chamber at a gas flow of 2.5 pm. Retro orbital injection of coelenterazine (4 mg/kg body weight) was administered and mice were placed in the anaesthesia manifold inside the imagining chamber and were imaged within 5 mins following substrate injection. Mice were placed into the light chamber and overlay images were collected for a period of 15 min. Images were then analysed using Living Image 4.0 software.

To address whether UCB-EC cells can exhibit similar anti-tumor effects in vivo, we tested the cytotoxic efficacy of UCB-EC cells against Gluc transduced SW480 cells in BRGS' mice. SW480 cells are EGFR⁺ RAS^{mut} and cetuximab monotherapy resistant. Previous study with UCB-EC cells in NSG mice reported in vivo upregulation of CD16 from 2% to 80% in 2 weeks⁸⁷, further in an effort to define, if that can translate into ADCC in vivo in BRGS' mice, combination therapy with cetuximab was proposed, although we didn't see benefits from UCB-EC+cetuximab studies in vitro (FIG. 30). The mice were divided into control groups (SW480 only and SW480+cetuximab) and treatment groups (SW480+UCB-EC and SW480+UCB-EC+cetuximab). 0.5×10⁶ Gluc SW480 cells were injected intravenously (i.v), followed by 30 million NK cells, infused as 10 million NK cells per injection (i.v) to UCB-EC only and UCB-EC+ cetuximab group and 0.5 mg cetuximab was injected intra-peritoneal (i.p) to the UCB-EC+ cetuximab group at days 1,4 and 7 post tumor injection. Bioluminescence imaging was done at day 35 to image tumor growth and as a measure to correlate with blood Gluc studies (FIG. 31). To assess the potential role of NK cells in controlling tumor growth and metastasis, we examined mice blood for Gaussia luciferase levels twice a week post tumor injection. Blood Gluc levels directly reflects tumor volume besides actively providing real time information on treatment significance⁸⁸. From blood Gluc levels, it was confirming that, growth of SW480 tumor cells, which were resistant to cetuximab in vitro, was not affected in vivo as well following treatment with cetuximab. However, interestingly, treatment with UCB-EC cells alone significantly decreased the tumor load in both treatment groups. The addition of cetuximab to UCB-EC cells did not have any effect in inhibiting RAS mutant tumor growth. Combining data, we observed that blood Gluc levels were significantly (p=0.013) reduced in the treatment groups compared to control groups (FIG. 32). Gluc measurements at different time points enabled longitudinal analysis of treatment clearly demonstrates the anti-tumor potential and suppression of systemic metastasis by adoptively transferred UCB-EC cells. These data are highly suggestive for use of UCB-EC cells in treating colon cancer, often in situations where antibody therapy is not effective.

### Example 16: UCB-EC Cells Effectively Targets and Lyse Cetuximab Resistant RAS Mutant Colon Cancer Cells In Vivo

### Target Cells Lentiviral Infection

EGFR⁺ RAS^{wt} A431 and EGFR⁺ RAS^{mut} SW480 cell lines were stably transduced with Gaussia Luciferase (Gluc) for in vivo studies. Lentiviral (LV) supernatants of Cerulean Fluorescent Protein (CFP) positive Gluc virus (LV-CFP-Gluc) was kindly provided by Thomas Wudringer, manufactured according to a protocol described in Wudringer et al., Nat Protoc. 2009; 4(4): 582-591)⁸⁶. Cells were sorted twice to achieve higher purity and transduction efficacy was checked using flow cytometry. SW480 cells with Gluc purity <95% were used for tumor injection in mice.

### Mice

Immunodeficient BRGS mice (BALB/c Rag2^{tm1Fwa} IL-2R_{γc}^{tm1Cgn} SIRPαNOD) were used in this study³. 24 adult mice were injected intravenously (i.v) via tail vein with 0.5×10⁶ SW480 Gluc cells and were randomized into 4 groups, SW480 only (I), SW480+cetuximab (II), SW480+UCB-EC (III) and SW480+UCB-EC+cetuximab (IV). 30×10⁶ UCB-EC were infused i.v per mice on days 1, 3 and 7 post tumor injection, 10×10⁶ cells per injection) for treatment groups III & IV. Similarly, 0.5 mg per mice cetuximab was injected intra peritoneal (i.p) for groups II & IV on days 1, 3 and 7. Treatment effects were monitored using blood Gluc levels and bioluminescence imaging (BLI). All manipulations of BRGS mice were performed under laminar flow conditions.

### Ethics Statement

Animals were housed in isolators under pathogen-free conditions with humane care and anesthesia was performed using inhalational isoflurane anaesthesia to minimize suffering. Experiments were approved by an ethical committee at the Institute Pasteur (Reference #2007-006) and validated by the French Ministry of Education and Research (Reference #02162.01).

### Bioluminescence Imaging

Mice were anesthetized with using isofluorane gas in an induction chamber at a gas flow of 2.5 pm. Retro orbital injection of coelenterazine (4 mg/kg body weight) was administered and mice were placed in the anaesthesia manifold inside the imagining chamber and were imaged within 5 mins following substrate injection. Mice were placed into the light chamber and overlay images were collected for a period of 15 min. Images were then analysed using Living Image 4.0 software.

While in vivo administration of UCB-EC cells were capable of reducing primary tumor load and metastasis from blood Gluc reading, next we imaged the mice to confirm the extent of tumor distribution and treatment efficacy. 4 mice were imaged from each group 35 days post tumor injection. Following tail vein injection of SW480 cells, 3 out of 4 mice presented with tumor overload detected initially in lungs, further spreading to liver, spleen, colon and abdominal cavity as shown in SW480 only and SW480+cetuximab groups, whereas in the treatment groups 3/4 and 2/4 were completely tumor free in UCB-EC only and UCB-EC+cetuximab groups, with significantly reduced radiance when compared to control groups (FIGS. 33A and 33B). Further, in parallel to SW480 experiments, and in order to verify if cetuximab is functional in vivo in BRGS^{wt} mice, anti-tumor effects of cetuximab were tested with a cetuximab sensitive, EGFR overexpressing RAS^{wt} A431 cell line. A significant decrease in tumor load was observed when A431 tumors were treated with the same concentration of cetuximab as SW480 cells (FIG. 33C). Overall the imaging result from SW480 studies correlated with blood Gluc studies, and in addition, there were no apparent difference between UCB-EC vs UCB-EC+cetuximab groups. Hence it is confirmed that cetuximab either as monotherapy or in combination with UCB-EC cells was unable to exert significant therapeutic benefits on RAS mutant tumors in vivo. These results further affirm to explore the use of UCB-EC cells as universal choice for treatment in mCRC patients resistant to cetuximab treatment.

### Example 17: UCB-EC Cells Treatment Reduces Tumor Growth and Increases Survival Rate In Vivo

### Target Cells Lentiviral Infection

EGFR⁺RAS^{wt} A431 and EGFR⁺ RAS^{mut} SW480 cell lines were stably transduced with Gaussia Luciferase (Gluc) for in vivo studies. Lentiviral (LV) supernatants of Cerulean Fluorescent Protein (CFP) positive Gluc virus (LV-CFP-Gluc) was kindly provided by Thomas Wudringer, manufactured according to a protocol described in Wudringer et al., Nat Protoc. 2009; 4(4): 582-591)⁸⁶. Cells were sorted twice to achieve higher purity and transduction efficacy was checked using flow cytometry. SW480 cells with Gluc purity <95% were used for tumor injection in mice.

### Mice

Immunodeficient BRGS mice (BALB/c Rag2^{tm1Fwa} IL-2R_{γc}^{tm1Cgn} SIRPαNOD) were used in this study⁴. 24 adult mice were injected intravenously (i.v) via tail vein with 0.5×10⁶ SW480 Gluc cells and were randomized into 4 groups, SW480 only (I), SW480+cetuximab (II), SW480+UCB-EC (III) and SW480+UCB-EC+cetuximab (IV). 30×10⁶ UCB-EC were infused i.v per mice on days 1, 3 and 7 post tumor injection, 10×10⁶ cells per injection) for treatment groups III & IV. Similarly, 0.5 mg per mice cetuximab was injected intra peritoneal (i.p) for groups II & IV on days 1, 3 and 7. Treatment effects were monitored using blood Gluc levels and bioluminescence imaging (BLI). All manipulations of BRGS mice were performed under laminar flow conditions.

### Ethics Statement

Animals were housed in isolators under pathogen-free conditions with humane care and anesthesia was performed using inhalational isoflurane anaesthesia to minimize suffering. Experiments were approved by an ethical committee at the Institute Pasteur (Reference #2007-006) and validated by the French Ministry of Education and Research (Reference #02162.01).

### Blood Gluc Quantification In Vitro

Secreted Gluc was measured according to a protocol previously described5. 10 μl of blood were collected by capillarity into EDTA containing Microvette® CB tubes. Blood samples were distributed in 96 well black plates and then mixed with 100 μl of 100 mM Gluc substrate native coelenterazine in PBS (P.J.K. GmbH; Kleinblittersdorf, Germany). Blood withdrawn before tumor inoculation served as a baseline value. Measurements were done twice a week till day 35. Gluc activity was measured using luminometer using the IVIS spectrum in vivo imaging system (PerkinElmer).

### Bioluminescence Imaging

Mice were anesthetized with using isofluorane gas in an induction chamber at a gas flow of 2.5 pm. Retro orbital injection of coelenterazine (4 mg/kg body weight) was administered and mice were placed in the anaesthesia manifold inside the imagining chamber and were imaged within 5 mins following substrate injection. Mice were placed into the light chamber and overlay images were collected for a period of 15 min. Images were then analysed using Living Image 4.0 software.

To address whether significant antitumor effect by UCB-EC cells can translate into survival advantage in vivo, the mice were monitored for survival benefits. Robust growth and spread of SW480 cells resulted in death of all PBS control mice by day 40. Mice treated with cetuximab survived till day 44 and no significant differences were observed between SW480 only and SW480+cetuximab groups. Treatment with UCB-EC cells alone resulted in a significant increase in survival by an additional 22 days (p=0.007) and 25 days (p=0.003) for UCB-EC+cetuximab compared to PBS control groups. Similarly, the data was significant comparing UCB-EC (p=0.0012) and UCB-EC+cetuximab (p=0.0015) to cetuximab only treatment groups. Survival among UCB-EC and UCB-EC+cetuximab did not differ significantly from one another.

Our results establish that UCB-EC cells efficiently target EGFR+RAS mutant tumors, thus facilitating increased survival in UCB-EC treated mice. The reported data, showing significant anti-tumor responses of UCB-EC cells, can be expanded to substantially improve the treatment outcomes in several other chemo refractory solid tumors.

### REFERENCES

1. Gratwohl, A. et al. Hematopoietic stem cell transplantation: a global perspective. Jama 303, 1617-1624 (2010).
2. Jansen, J. The first successful allogeneic bone-marrow transplant: Georges Mathe. Transfusion medicine reviews 19, 246-248 (2005).
3. Korbling, M. & Anderlini, P. Peripheral blood stem cell versus bone marrow allotransplantation: does the source of hematopoietic stem cells matter? Blood 98, 2900-2908 (2001).
4. Copelan, E. A. Hematopoietic stem-cell transplantation. N Engl J Med 354, 1813-1826 (2006).
5. Jenq, R. R. & van den Brink, M. R. Allogeneic haematopoietic stem cell transplantation: individualized stem cell and immune therapy of cancer. Nat Rev Cancer 10, 213-221 (2010).
6. Ruggeri, L. et al. Effectiveness of donor natural killer cell alloreactivity in mismatched hematopoietic transplants. Science 295, 2097-2100 (2002).
7. Olson, J. A. et al. NK cells mediate reduction of GVHD by inhibiting activated, alloreactive T cells while retaining GVT effects. Blood 115, 4293-4301 (2010).
8. Tanaka, M. et al. The impact of the dose of natural killer cells in the graft on severe acute graft-versus-host disease after unrelated bone marrow transplantation. Leukemia research 36, 699-703 (2012).
9. Farag, S. S. & Caligiuri, M. A. Cytokine modulation of the innate immune system in the treatment of leukemia and lymphoma. Adv Pharmacol 51, 295-318 (2004).
10. Rosenberg, S. A. Interleukin-2 and the development of immunotherapy for the treatment of patients with cancer. Cancer J Sci Am 6 Suppl 1, S2-7 (2000).
11. Fehniger, T. A., Cooper, M. A. & Caligiuri, M. A. Interleukin-2 and interleukin-15: immunotherapy for cancer. Cytokine Growth Factor Rev 13, 169-183 (2002).
12. Waldmann, T. The contrasting roles of IL-2 and IL-15 in the life and death of lymphocytes: implications for the immunotherapy of rheumatological diseases. Arthritis Res 4 Suppl 3, S161-167 (2002).
13. NIH (2012).
14. NIH http://clinicaltrials.gov/ct2/show?term=IL-15&rank=1. (2012).
15. Coiffier, B. et al. CHOP chemotherapy plus rituximab compared with CHOP alone in elderly patients with diffuse large-B-cell lymphoma. N Engl J Med 346, 235-242 (2002).
16. Marcus, R. et al. CVP chemotherapy plus rituximab compared with CVP as first-line treatment for advanced follicular lymphoma. Blood 105, 1417-1423 (2005).
17. Benson, D. M., Jr. et al. IPH2101, a novel anti-inhibitory KIR antibody, and lenalidomide combine to enhance the natural killer cell versus multiple myeloma effect. Blood 118, 6387-6391 (2011).
18. Lundqvist, A., Berg, M., Smith, A. & Childs, R. W. Bortezomib Treatment to Potentiate the Anti-tumor Immunity of Ex-vivo Expanded Adoptively Infused Autologous Natural Killer Cells. J Cancer 2, 383-385 (2011).
19. Marin, D. et al. KIR2DS1 genotype predicts for complete cytogenetic response and survival in newly diagnosed chronic myeloid leukemia patients treated with imatinib. Leukemia (2011).
20. Terme, M., Ullrich, E., Delahaye, N. F., Chaput, N. & Zitvogel, L. Natural killer cell-directed therapies: moving from unexpected results to successful strategies. Nature immunology 9, 486-494 (2008).
21. Shah, N. N. et al. Acute GVHD in patients receiving IL-15/4-1BBL activated NK cells following T-cell-depleted stem cell transplantation. Blood 125, 784-792 (2015).
22. Miller, J. S. et al. Successful adoptive transfer and in vivo expansion of human haploidentical NK cells in patients with cancer. Blood 105, 3051-3057 (2005).
23. Porter, D. L. et al. Chimeric antigen receptor T cells persist and induce sustained remissions in relapsed refractory chronic lymphocytic leukemia. Sci Transl Med 7, 303ra139 (2015).
24. Liu, X. et al. Affinity-Tuned ErbB2 or EGFR Chimeric Antigen Receptor T Cells Exhibit an Increased Therapeutic Index against Tumors in Mice. Cancer Res 75, 3596-3607 (2015).
25. Kenderian, S. S. et al. CD33-specific chimeric antigen receptor T cells exhibit potent preclinical activity against human acute myeloid leukemia. Leukemia 29, 1637-1647 (2015).
26. Garfall, A. L. et al. Chimeric Antigen Receptor T Cells against CD19 for Multiple Myeloma. N Engl J Med 373, 1040-1047 (2015).
27. Frigault, M. J. et al. Identification of chimeric antigen receptors that mediate constitutive or inducible proliferation of T cells. Cancer Immunol Res 3, 356-367 (2015).
28. Barrett, D. M., Grupp, S. A. & June, C. H. Chimeric Antigen Receptor- and TCR-Modified T Cells Enter Main Street and Wall Street. J Immunol 195, 755-761 (2015).
29. Therasse, P. et al. New Guidelines to Evaluate the Response to Treatment in Solid Tumors. Journal of the National Cancer Institute 92, 205-216 (2000).
30. Trottier, H. & Franco, E. L. The epidemiology of genital human papillomavirus infection. Vaccine 24 Suppl 1, S1-15 (2006).
31. Woodman, C. B., Collins, S. I. & Young, L. S. The natural history of cervical HPV infection: unresolved issues. Nat Rev Cancer 7, 11-22 (2007).
32. Zagouri, F., Sergentanis, T. N., Chrysikos, D., Filipits, M. & Bartsch, R. Molecularly targeted therapies in cervical cancer. A systematic review. Gynecologic oncology 126, 291-303 (2012).
33. Diaz-Padilla, I., Monk, B. J., Mackay, H. J. & Oaknin, A. Treatment of metastatic cervical cancer: Future directions involving targeted agents. Critical Reviews in Oncology/Hematology 85, 303-314 (2013).
34. Li, Q. et al. EGFR protein expression and gene amplification in squamous intraepithelial lesions and squamous cell carcinomas of the cervix. International Journal of Clinical and Experimental Pathology 7, 733-741 (2014).
35. Kersemaekers, A. M. et al. Oncogene alterations in carcinomas of the uterine cervix: overexpression of the epidermal growth factor receptor is associated with poor prognosis. Clin Cancer Res 5, 577-586 (1999).
36. Schrevel, M. et al. Molecular mechanisms of epidermal growth factor receptor overexpression in patients with cervical cancer. Modern pathology: an official journal of the United States and Canadian Academy of Pathology, Inc 24, 720-728 (2011).
37. Hertlein, L. et al. Cetuximab monotherapy in advanced cervical cancer: a retrospective study with five patients. Arch Gynecol Obstet 283, 109-113 (2011).
38. Menderes, G., Black, J., Schwab, C. L. & Santin, A. D. Immunotherapy and targeted therapy for cervical cancer: an update. Expert review of anticancer therapy 16, 83-98 (2016).
39. Wang, D. et al. Indoleamine-2,3-dioxygenase, an immunosuppressive enzyme that inhibits natural killer cell function, as a useful target for ovarian cancer therapy. International journal of oncology 40, 929-934 (2012).
40. Jordanova, E. S. et al. Human leukocyte antigen class I, MHC class I chain-related molecule A, and CD8+/regulatory T-cell ratio: which variable determines survival of cervical cancer patients? Clin Cancer Res 14, 2028-2035 (2008).
41. Kobayashi, A. et al. Functional attributes of mucosal immunity in cervical intraepithelial neoplasia and effects of HIV infection. Cancer Res 64 (2004).
42. Textor, S. et al. Activating NK cell receptor ligands are differentially expressed during progression to cervical cancer. Int J Cancer 123 (2008).
43. Arreygue-Garcia, N. A. et al. Augmented serum level of major histocompatibility complex class I-related chain A (MICA) protein and reduced NKG2D expression on NK and T cells in patients with cervical cancer and precursor lesions. BMC Cancer 8 (2008).
44. Garcia-Iglesias, T. et al. Low NKp30, NKp46 and NKG2D expression and reduced cytotoxic activity on NK cells in cervical cancer and precursor lesions. BMC Cancer 9 (2009).
45. Spaans, V. M., Peters, A. A., Fleuren, G. J. & Jordanova, E. S. HLA-E expression in cervical adenocarcinomas: association with improved long-term survival. J Transl Med 10, 184 (2012).
46. Cho, H. et al. MICA/B and ULBP1 NKG2D ligands are independent predictors of good prognosis in cervical cancer. BMC Cancer 14, 957 (2014).
47. Connor, M. E. & Stern, P. L. Loss of MHC class-I expression in cervical carcinomas. Int J Cancer 46, 1029-1034 (1990).
48. Jemal, A. et al. Global cancer statistics. CA: A Cancer Journal for Clinicians 61, 69-90 (2011).
49. Siegel, R. L., Miller, K. D. & Jemal, A. Cancer statistics, 2016. CA Cancer J Clin 66, 7-30 (2016).
50. Qiu, M., Hu, J., Yang, D., Cosgrove, D. P. & Xu, R. Pattern of distant metastases in colorectal cancer: a SEER based study. Oncotarget 6, 38658-38666 (2015).
51. Petrelli, F., Borgonovo, K., Cabiddu, M., Ghilardi, M. & Barni, S. Cetuximab and panitumumab in KRAS wild-type colorectal cancer: a meta-analysis. International journal of colorectal disease 26, 823-833 (2011).
52. Karapetis, C. et al. K-ras mutations and benefit from cetuximab in advanced colorectal cancer. N Engl J Med 359, 1757-1765 (2008).
53. Boeckx, C. et al. Overcoming cetuximab resistance in HNSCC: the role of AURKB and DUSP proteins. Cancer letters 354, 365-377 (2014).
54. Dienstmann, R., Salazar, R. & Tabernero, J. Overcoming Resistance to Anti-EGFR Therapy in Colorectal Cancer. American Society of Clinical Oncology educational book/ASCO. American Society of Clinical Oncology. Meeting, e149-156 (2015).
55. Schirripa, M. & Lenz, H.-J. Colorectal cancer: Overcoming resistance to anti-EGFR therapy [mdash] where do we stand? Nat Rev Gastroenterol Hepatol 13, 258-259 (2016).
56. Deschoolmeester, V., Baay, M., Lardon, F., Pauwels, P. & Peeters, M. Immune Cells in Colorectal Cancer: Prognostic Relevance and Role of MSI. Cancer Microenvironment 4, 377-392 (2011).
57. Koido, S. et al. Immunotherapy for colorectal cancer. World Journal of Gastroenterology: WJG 19, 8531-8542 (2013).
58. Wang, M., Yin, B., Wang, H. Y. & Wang, R.-F. Current advances in T-cell-based cancer immunotherapy. Immunotherapy 6, 1265-1278 (2014).
59. Parkhurst, M. R. et al. T cells targeting carcinoembryonic antigen can mediate regression of metastatic colorectal cancer but induce severe transient colitis. Molecular therapy: the journal of the American Society of Gene Therapy 19, 620-626 (2011).
60. Morgan, R. A. et al. Case report of a serious adverse event following the administration of T cells transduced with a chimeric antigen receptor recognizing ERBB2. Molecular therapy: the journal of the American Society of Gene Therapy 18, 843-851 (2010).
61. Ljunggren, H. G. & Karre, K. In search of the ‘missing self’: MHC molecules and NK cell recognition. Immunology today 11, 237-244 (1990).
62. Sandel, M. H. et al. Natural killer cells infiltrating colorectal cancer and MHC class I expression. Molecular immunology 42, 541-546 (2005).
63. Vivier, E., Tomasello, E., Baratin, M., Walzer, T. & Ugolini, S. Functions of natural killer cells. Nature immunology 9, 503-510 (2008).
64. Larsen, S. K., Gao, Y. & Basse, P. H. NK Cells in the Tumor Microenvironment. Critical reviews in oncogenesis 19, 91-105 (2014).
65. Parkhurst, M. R., Riley, J. P., Dudley, M. E. & Rosenberg, S. A. Adoptive transfer of autologous natural killer cells leads to high levels of circulating natural killer cells but does not mediate tumor regression. Clin Cancer Res 17, 6287-6297 (2011).
66. deMagalhaes-Silverman, M. et al. Posttransplant adoptive immunotherapy with activated natural killer cells in patients with metastatic breast cancer. Journal of immunotherapy (Hagerstown, Md.: 1997) 23, 154-160 (2000).
67. Ishikawa, E. et al. Autologous natural killer cell therapy for human recurrent malignant glioma. Anticancer Res 24, 1861-1871 (2004).
68. Veluchamy, J. P. et al. Combination of NK Cells and Cetuximab to Enhance Anti-Tumor Responses in RAS Mutant Metastatic Colorectal Cancer. PLoS ONE 11, e0157830 (2016).
69. Campbell, K. S. & Hasegawa, J. Natural killer cell biology: an update and future directions. J Allergy Clin Immunol 132, 536-544 (2013).
70. Geller, M. A. & Miller, J. S. Use of allogeneic NK cells for cancer immunotherapy. Immunotherapy 3, 1445-1459 (2011).
71. Geller, M. A. et al. A phase II study of allogeneic natural killer cell therapy to treat patients with recurrent ovarian and breast cancer. Cytotherapy 13, 98-107 (2011).
72. Spanholtz, J. et al. Clinical-grade generation of active NK cells from cord blood hematopoietic progenitor cells for immunotherapy using a closed-system culture process. PLoS One 6, e20740 (2011).
73. Lehmann, D. et al. Ex vivo generated natural killer cells acquire typical natural killer receptors and display a cytotoxic gene expression profile similar to peripheral blood natural killer cells. Stem cells and development 21, 2926-2938 (2012).
74. Lee, N. et al. HLA-E is a major ligand for the natural killer inhibitory receptor CD94/NKG2A. Proceedings of the National Academy of Sciences of the United States of America 95, 5199-5204 (1998).
75. Gonen-Gross, T. et al. The CD85J/leukocyte inhibitory receptor-1 distinguishes between conformed and beta 2-microglobulin-free HLA-G molecules. J Immunol 175, 4866-4874 (2005).
76. Spanholtz, J. et al. High log-scale expansion of functional human natural killer cells from umbilical cord blood CD34-positive cells for adoptive cancer immunotherapy. PLoS One 5, e9221 (2010).
77. Eissens, D. N. et al. Defining early human NK cell developmental stages in primary and secondary lymphoid tissues. PLoS One 7, e30930 (2012).
78. Feller, N. et al. Defining consensus leukemia-associated immunophenotypes for detection of minimal residual disease in acute myeloid leukemia in a multicenter setting. Blood Cancer J 3, e129 (2013).
79. Koopman, L. A. et al. Recurrent integration of human papillomaviruses 16, 45, and 67 near translocation breakpoints in new cervical cancer cell lines. Cancer Res 59, 5615-5624 (1999).
80. Santin, A. D. et al. Phase II trial of cetuximab in the treatment of persistent or recurrent squamous or non-squamous cell carcinoma of the cervix: a Gynecologic Oncology Group study. Gynecologic oncology 122, 495-500 (2011).
81. Inaba, T. et al. Indoleamine 2,3-dioxygenase expression predicts impaired survival of invasive cervical cancer patients treated with radical hysterectomy. Gynecologic oncology 117, 423-428 (2010).
82. Sedlmayr, P. et al. Expression of indoleamine 2,3-dioxygenase in carcinoma of human endometrium and uterine cervix. Adv Exp Med Biol 527 (2003).
83. Nakamura, T. et al. Expression of indoleamine 2, 3-dioxygenase and the recruitment of Foxp3-expressing regulatory T cells in the development and progression of uterine cervical cancer. Cancer Sci 98, 874-881 (2007).
84. Sato, N. et al. Downregulation of indoleamine-2,3-dioxygenase in cervical cancer cells suppresses tumor growth by promoting natural killer cell accumulation. Oncology reports 28, 1574-1578 (2012).
85. Garcia-Iglesias, T. et al. Low NKp30, NKp46 and NKG2D expression and reduced cytotoxic activity on NK cells in cervical cancer and precursor lesions. BMC Cancer 9, 1-8 (2009).
86. Wurdinger, T., Badr, C. & Tannous, B. Gaussia luciferase blood level as an index of cell growth and proliferation. (2008).
87. Cany, J. et al. Natural Killer Cells Generated from Cord Blood Hematopoietic Progenitor Cells Efficiently Target Bone Marrow-Residing Human Leukemia Cells in NOD/SCID/IL2Rg<sup>null</sup> Mice. PLoS ONE 8, e64384 (2013).
88. Chung, E. et al. Secreted Gaussia Luciferase as a Biomarker for Monitoring Tumor Progression and Treatment Response of Systemic Metastases. PLoS ONE 4, e8316 (2009).

1. A method of providing immunotherapy treatment to an individual, the method comprising administering a composition comprising an immune effector cell wherein the immune effector cell is non-haploidentical with respect to the individual.

2. The method according to claim 1, wherein the immune effector cell is positive for Neural Cell Adhesion Molecule (NCAM) and negative for CD3 and CD19.

3. The method according to claim 1, wherein the immune effector cell expresses one or more of the following cell surface markers: CD159a, CD314, CD335, CD336, CD337.

4. The method according to claim 1, wherein the immune effector cell expresses at least one of CD314 or CD336.

5. The method according to claim 1, wherein the composition further comprises a plurality of cells, characterized in that 40-100% of the plurality of cells is the immune effector cell.

6. The method according to claim 1, wherein the immunotherapy is for the treatment of a tumor.

7. The method according to claim 1, wherein the immune effector cell is generated ex vivo from a stem cell or a progenitor cell.

8. The method according to claim 7, wherein the stem cell or the progenitor cell is a CD34+ stem cell or CD34+ progenitor cell, respectively.

9. The method according to claim 5, wherein the plurality of cells are derived from cells obtained from a single donor.

10. The method according to claim 5, wherein the plurality of cells are derived from at least one of umbilical cord blood and bone marrow.

11. The method according to claim 1, wherein the composition is generated ex vivo in a process comprising the steps of:
a) obtaining a sample comprising at least one of CD34+ hematopoietic stem or progenitor cells;
b) performing affinity purification of at least one of or both CD34+ hematopoietic stem or progenitor cells from the sample obtained in a);
c) expanding at least one of or both the purified CD34+ hematopoietic stem or progenitor cells obtained in b) in a basal growth medium supplemented with human serum, a low-dose cytokine cocktail consisting of three or more GM-CSF, G-CSF, LIF, MIP-lα and IL-6, a specific combination of two or more of high-dose cytokines including SCF, Flt3L, IL-7 and TPO and a low-molecular weight heparin; and,
d) differentiating at least one of or both the expanded CD34+ hematopoietic stem or progenitor cells obtained in c) in a basal growth medium supplemented with human serum and IL-15 and additional one or more cytokines including SCF, Flt3L, IL-7, IL-12, IL-18 and IL-2,
e) harvesting the cells generated in d) and generating the composition of claim 1.

12. The method according to claim 1, further comprising administering cyclophosphamide prior to administering of the composition, wherein the cyclophosphamide is dosed on 2, 3, 4 or 5 subsequent days at a total dose of 400-10000 mg/m² concomitant with fludarabine at a total dose of 1-1000 mg/m².

13. The method according to claim 1, wherein the composition to be administered in one treatment comprises at least 5×10⁸ cells.

14. The method according to claim 1, wherein the composition to be administered in one treatment comprises not more than 1×10¹⁰ cells.

15. The method according to claim 6, wherein the tumor is a haematopoietic or lymphoid tumor or wherein the tumor is a solid tumor.

16. The method according to claim 6, wherein the tumor is a haematopoietic or lymphoid tumor, selected from leukemia, lymphoma, myelodysplastic syndrome, or myeloma.

17. The method according to claim 16, wherein the leukemia is AML.

18. The method according to claim 6, wherein the tumor is a solid tumor, selected from malignant neoplasms or mestastatic induced secondary tumors of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumor, Waldenstrom macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma, or retinoblastoma.

19. The method according to claim 6, wherein the tumor is a solid tumor that is selected from malignant neoplasms or metastatic induced secondary tumors of cervical cancers selected from adenocarcinoma, squamous cell carcinoma, adenosquamous carcinoma, cervix carcinoma, small cell carcinoma, and melanoma.

20. The method according to claim 6, wherein the tumor is a solid tumor that is selected from malignant neoplasms or metastatic induced secondary tumors of colorectal cancers selected from adenocarcinoma, squamous cell carcinoma, colon adenocarcinoma, colorectal carcinoma, colorectal adenocarcinoma, colon carcinoma, and melanoma.
